(19) United States
(12) Patent Application Publication
(10) Pub. No.: US 2015/0179942 A1
(43) Pub. Date: Jun. 25, 2015

(54) MATERIAL FOR ORGANIC ELECTROLUMINESCENCE DEVICE AND ORGANIC ELECTROLUMINESCENCE DEVICE INCLUDING THE SAME

(71) Applicant: SAMSUNG DISPLAY CO., LTD., Yongin-City (KR)

(72) Inventor: Yasuo MIYATA, Yokohama (JP)

(21) Appl. No.: 14/576,989

(22) Filed: Dec. 19, 2014

(30) Foreign Application Priority Data
Dec. 20, 2013 (JP) 2013-263851

(51) Int. Cl. H01L 51/00; C07D 307/91; H01L 51/50; C07F 7/10; C07C 255/58; C07D 487/04; C07C 211/61; C07D 209/86

(52) U.S. Cl. CPC H01L 51/006; C07C 211/61; C07D 307/91; H01L 51/0061; C07D 209/86; C07F 7/10; H01L 51/0094; C07C 255/58; C07D 487/04; H01L 51/5056; H01L 51/5012; H01L 51/0052; H01L 51/0058; H01L 51/0073; H01L 51/0072; H01L 51/5088

(57) ABSTRACT

A material for an organic electroluminescence (EL) device is represented by the following Formula (1):

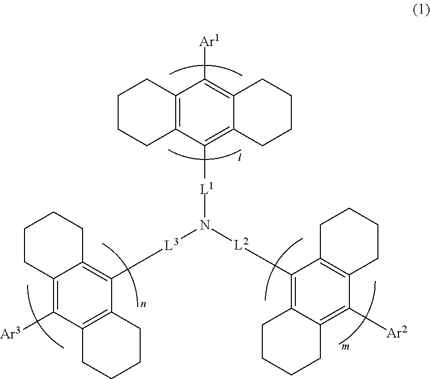

where Ar¹, Ar² and Ar³, L¹, L² and L³, and l, m and n are as defined in the specification.

### CROSS-REFERENCE TO RELATED APPLICATION

Japanese Patent Application No. 2013-263851, filed on Dec. 20, 2013, in the Japanese Patent Office, and entitled: “Material for Organic Electroluminescence Device and Organic Electroluminescence Device Including the Same,” is incorporated by reference herein in its entirety.

### BACKGROUND

1. Field

Embodiments relate to a material for an organic electroluminescence device and an organic electroluminescence device including the same.

2. Description of the Related Art

In recent years, organic electroluminescence (EL) displays, which are one type of image display, have been actively developed. Unlike a liquid crystal display or the like, the organic EL display is a self-luminescent display. In the organic EL display, holes and electrons injected from an anode and a cathode are recombined in an emission layer such that a light-emitting material including an organic compound of the emission layer emits light, thereby providing a display.

### SUMMARY

Embodiments are directed to a material for an organic electroluminescence (EL) device represented by the following Formula (1):

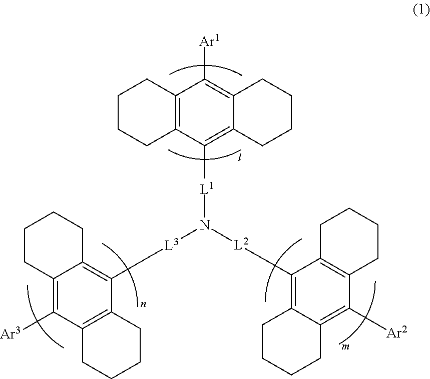

where Ar¹, Ar² and Ar³ are independently a hydrogen atom, a deuterium atom, a halogen atom, a cyano group, a substituted or unsubstituted alkyl group having 1 to 30 carbon atoms, a substituted or unsubstituted alkoxy group having 1 to 30 carbon atoms, a substituted or unsubstituted aryl group having 6 to 30 ring carbon atoms, a substituted or unsubstituted heteroaryl group having 5 to 30 ring atoms, a substituted or unsubstituted triarylsilyl group, or a substituted or unsubstituted trialkylsilyl group, l, m and n are independently 0 or 1, where the relation of l+m+n≧1 is satisfied, and L¹, L² and L³ are independently a single bond, a substituted or unsubstituted aryl group having 6 to 30 ring carbon atoms, or a substituted or unsubstituted heteroaryl group having 5 to 30 ring atoms.

The relation of l+m+n=1 may be satisfied with respect to l, m and n.

Ar¹, Ar² and Ar³ may be independently a hydrogen atom, a substituted or unsubstituted aryl group having 6 to 18 ring carbon atoms, a substituted or unsubstituted carbazolyl group, a substituted or unsubstituted benzofuryl group or a substituted or unsubstituted dibenzothienyl group.

The relation of l+m+n=1 may be satisfied with respect to l, m and n.

L¹, L² and L³ may be independently the single bond or an aryl group having 6 to 18 ring carbon atoms.

Embodiments are also directed to an organic electroluminescence (EL) device including the material for an organic EL.

Embodiments are also directed to an organic electroluminescence (EL) device including the material for an organic EL device in a layer of stacked layers between an emission layer and an anode.

### BRIEF DESCRIPTION OF THE DRAWING

Features will become apparent to those of skill in the art by describing in detail exemplary embodiments with reference to the attached drawing in which:

FIG. 1 illustrates a schematic diagram depicting an organic EL device 100 according to an embodiment.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figure, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “between” two layers, it can be the only layer between the two layers, or one or more intervening layers may also be present.

According to an embodiment, the driving at a low voltage, the high emission efficiency and the long life of an organic EL device may be realized by introducing an octahydroanthracene group in an amine compound. The material for an organic EL device may be included in a layer of stacked layers disposed between an anode and an emission layer. Remarkable effects may be obtained in a blue emission region and a green emission region.

Hereinafter, the material for an organic EL device and an organic EL device using the same will be explained referring to attached drawing. The material for an organic EL device and the organic EL device using the same may, however, be embodied in different forms and should not be construed as limited to the embodiments set forth herein. In the drawing, like reference numerals refer to like elements having substantially the same function throughout, and repeated explanation thereof will be omitted.

The material for an organic EL device may include an amine compound containing at least one octahydroanthracene group, the amine compound being represented by the following Formula (1).

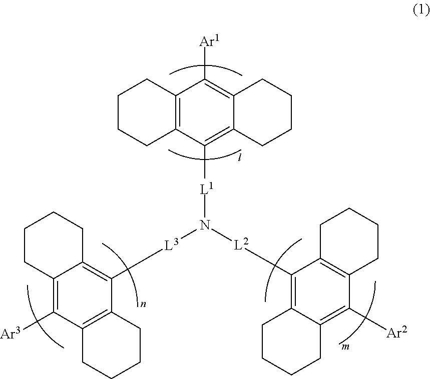

In Formula (1), Ar¹, Ar² and Ar³ are independently a hydrogen atom, a deuterium atom, a halogen atom, a cyano group, a substituted or unsubstituted alkyl group having 1 to 30 carbon atoms, a substituted or unsubstituted alkoxy group having 1 to 30 carbon atoms, a substituted or unsubstituted aryl group having 6 to 30 ring carbon atoms, a substituted or unsubstituted heteroaryl group having 5 to 30 ring atoms, a substituted or unsubstituted triarylsilyl group, or a substituted or unsubstituted trialkylsilyl group, and 1, m and n are independently 0 or 1, where the relation of l+m+n≧1 is satisfied. L¹, L² and L³ are independently a single bond, a substituted or unsubstituted aryl group having 6 to 30 ring carbon atoms, or a substituted or unsubstituted heteroaryl group having 5 to 30 ring atoms.

In the material for an organic EL device, 1, m and n may satisfy the relation of l+m+n=1. According to an embodiment, the driving at a low voltage, the high emission efficiency and the long life of the organic EL device may be realized by introducing an octahydroanthracene group in an amine compound. In some implementations, the material for an organic EL device may include two or three octahydroanthracene groups in the amine compound, and the octahydroanthracene group may be introduced in one of l, m or n.

By introducing at least one octahydroanthracene group in the amine compound in the organic EL device, driving at a low voltage, high emission efficiency and long life of the organic EL device may be realized.

The halogen atom referred to with respect to Ar¹, Ar² and Ar³ in Formula (1) may include a fluorine atom, a chlorine atom, a bromine atom and an iodine atom.

The alkyl group of the “substituted or unsubstituted alkyl group having 1 to 30 carbon atoms” referred to with respect to Ar¹, Ar² and Ar³ in Formula (1) may include a methyl group, an ethyl group, a n-propyl group, an isopropyl group, a n-butyl group, an s-butyl group, a t-butyl group, an i-butyl group, a 2-ethylbutyl group, a 3,3-dimethylbutyl group, a n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, a n-hexyl group, a 1-methylhexyl group, a 2-ethylhexyl group, a 2-butylhexyl group, a cyclohexyl group, a 4-methylcyclohexyl group, a 4-t-butylcyclohexyl group, a n-heptyl group, a 1-methylheptyl group, a 2,2-dimethylheptyl group, a 2-ethylheptyl group, a 2-butylheptyl group, a n-octyl group, a t-octyl group, a 2-ethyloctyl group, a 2-butyloctyl group, a 2-hexyloctyl group, a 3,7-dimethyloctyl group, a cyclooctyl group, a n-nonyl group, a n-decyl group, an adamantly group, a 2-ethyldecyl group, a 2-butyldecyl group, a 2-hexyldecyl group, a 2-octyldecyl group, a n-undecyl group, a n-dodecyl group, a 2-ethyldocecyl group, a 2-butyldodecyl group, a 2-hexyldodecyl group, a 2-octyldodecyl group, a n-tridecyl group, a n-tetradecyl group, a n-pentadecyl group, a n-hexadecyl group, a 2-ethylhexadecyl group, a 2-butylhexadecyl group, a 2-hexylhexadecyl group, a 2-octylhexadecyl group, a n-heptadecyl group, a n-octadecyl group, a n-nonadecyl group, a n-icosyl group, a 2-ethylicosyl group, a 2-butylicosyl group, a 2-hexylicosyl group, a 2-octylicosyl group, a 2-hexylicosyl group, a 2-octylicosyl group, a n-henicosyl group, a n-docosyl group, a n-tricosyl group, a n-tetracosyl group, a n-pentacosyl group, a n-hexacosyl group, a n-heptacosyl group, a n-octacosyl group, a n-nonacosyl group, a n-triacontyl group, etc.

The alkoxy group of the “substituted or unsubstituted alkoxy group having 1 to 30 carbon atoms” may include a methoxy group, an ethoxy group, a n-propoxy group, an i-propoxy group, a n-butoxy group, an s-butoxy group, a t-butoxy group, an i-butoxy group, a 2-ethylbutoxy group, a 3,3-dimethylbutoxy group, a n-pentyloxy group, an i-pentyloxy group, a neopentyloxy group, a t-pentyloxy group, a cyclopentyloxy group, a 1-methylpentyloxy group, a 3-methylpentyloxy group, a 2-ethylpentyloxy group, a 4-methyl-2-pentyloxy group, a n-hexyloxy group, a 1-methylhexyloxy group, a 2-ethylhexyloxy group, a 2-butylhexyloxy group, a cyclohexyloxy group, a 4-methylcyclohexyloxy group, a 4-t-butylcyclohexyloxy group, a n-heptyloxy group, a 1-methylheptyloxy group, a 2,2-dimethylheptyloxy group, a 2-ethylheptyloxy group, 2-butylheptyloxy group, a n-octyloxy group, a t-octyloxy group, a 2-ethyloctyloxy group, a 2-butyloctyloxy group, a 2-hexyloctyloxy group, a 3,7-dimethyloctyloxy group, a cyclooctyloxy group, a n-nonyloxy group, a n-decyloxy group, an adamantyloxy group, a 2-ethyldecyloxy group, a 2-butyldecyloxy group, a 2-hexyldecyloxy group, a 2-octyldecyloxy group, a n-undecyloxy group, a n-dodecyloxy group, a 2-ethyldodecyloxy group, a 2-butyldodecyloxy group, a 2-hexyldodecyloxy group, a 2-octyldodecyloxy group, a n-tridecyloxy group, a n-tetradecyloxy group, a n-pentadecyloxy group, a n-hexadecyloxy group, a 2-ethylhexadecyloxy group, a 2-butylhexadecyloxy group, a 2-hexylhexadecyloxy group, a 2-octylhexadecyloxy group, a n-heptadecyloxy group, a n-octadecyloxy group, a n-nonadecyloxy group, a n-icosyloxy group, a 2-ethylicosyloxy group, a 2-butylicosyloxy group, a 2-hexylicosyloxy group, a 2-octylicosyloxy group, a n-henicosyloxy group, a n-docosyloxy group, a n-tricosyloxy group, a n-tetracosyloxy group, a n-pentacosyloxy group, a n-hexacosyloxy group, a n-heptacosyloxy group, a n-octacosyloxy group, a n-nonacosyloxy group, a n-triacontyloxy group, etc.

The aryl group of the “substituted or unsubstituted aryl group having 6 to 30 ring carbon atoms” may include a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylene group, a biphenylene group, a pyrenyl group, a benzofluoranthenyl group, a chrysenyl group, etc.

The heteroaryl group of the “substituted or unsubstituted heteroaryl group having 5 to 30 ring atoms” may include a benzothiazolyl group, a thiophenyl group, a thienothiophenyl group, a thienothienothiophenyl group, a benzothiophenyl group, a benzofuryl group, a dibenzothiophenyl group, a benzofuryl group, an N-arylcarbazolyl group, an N-heteroarylcarbazolyl group, an N-alkylcarbazolyl group, a phenoxazyl group, a phenothiazyl group, a pyridyl group, a pyrimidyl group, a triazinyl group, a quinolinyl group, a quinoxalyl group, etc.

The “aryl group” of the “substituted or unsubstituted triarylsilyl group” may be the same as the above described “aryl group.”

The “alkyl group” of the “substituted or unsubstituted trialkylsilyl group” may be the same as the above described “alkyl group.”

In an embodiment, Ar¹, Ar² and Ar³ in Formula (1) may be a hydrogen atom, a substituted or unsubstituted aryl group having 6 to 18 ring carbon atoms, a substituted or unsubstituted carbazolyl group, a substituted or unsubstituted benzofuryl group or a substituted or unsubstituted dibenzothienyl group. In some implementations, a phenyl group, a naphthyl group, a anthracenyl group, a phenanthryl group, a biphenyl group, a terphenyl group, a fluorenyl group, a triphenylene group, a pyrenyl group, a benzofluoranthenyl group or a chrysenyl group may be used as the aryl group used as Ar¹, Ar² and Ar³ of the material for an organic EL device.

By introducing the above-described substituents in an amine compound via at least one octahydroanthracene group in the material for an organic EL device, 1 driving at a low voltage, 1 high emission efficiency 1 the long life of the organic EL device may be realized.

In the definition of L¹, L² and L³ in the above Formula (1), the aryl group of the substituted or unsubstituted aryl group having 6 to 30 ring carbon atoms may be the same as the above described “aryl group.”

The heteroaryl group of the substituted or unsubstituted heteroaryl group having 5 to 30 ring atoms may be the group as the above described “heteroaryl group.”

In an embodiment, L¹, L² and L³ in the above Formula (1) may be a single bond, or a substituted or unsubstituted aryl group having 6 to 18 ring carbon atoms. The aryl group may be a phenyl group, a naphthyl group, an anthracenyl group, a phenanthryl group, a biphenyl group, a terphenyl group, a fluorenyl group, a triphenylene group, a pyrenyl group, a benzofluoranthenyl group or a chrysenyl group, as examples.

In the organic EL device, driving at a low voltage, high emission efficiency and long life of the organic EL device may be realized by introducing at least one octahydroanthracene group in an amine compound via a single bond or the above described connecting group.

The material for an organic EL device may be represented by one or more of the following structures.

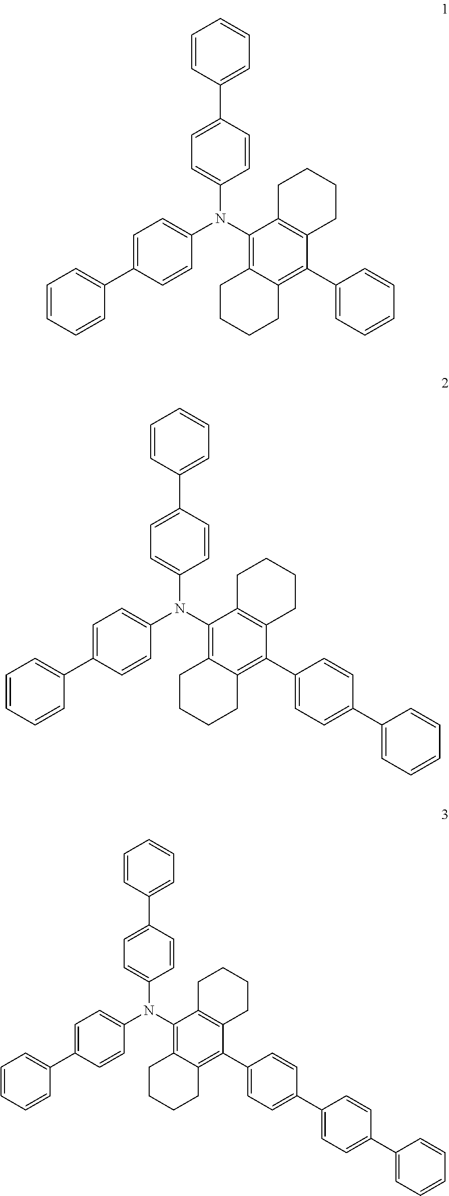 [SMILES: c1ccc(-c2ccc(N(c3ccc(-c4ccccc4)cc3)c3c4c(c(-c5ccccc5)c5c3CCCC5)CCCC4)cc2)cc1 ; c1ccc(-c2ccc(-c3c4c(c(N(c5ccc(-c6ccccc6)cc5)c5ccc(-c6ccccc6)cc5)c5c3CCCC5)CCCC4)cc2)cc1 ; c1ccc(-c2ccc(-c3ccc(-c4c5c(c(N(c6ccc(-c7ccccc7)cc6)c6ccc(-c7ccccc7)cc6)c6c4CCCC6)CCCC5)cc3)cc2)cc1]

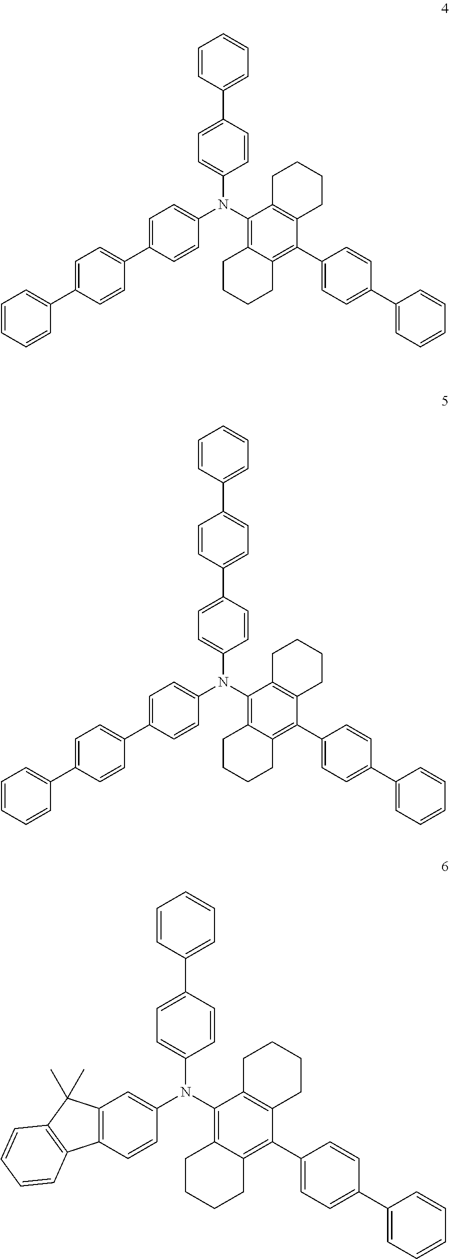 [SMILES: c1ccc(-c2ccc(-c3ccc(N(c4ccc(-c5ccccc5)cc4)c4c5c(c(-c6ccc(-c7ccccc7)cc6)c6c4CCCC6)CCCC5)cc3)cc2)cc1 ; c1ccc(-c2ccc(-c3ccc(N(c4ccc(-c5ccc(-c6ccccc6)cc5)cc4)c4c5c(c(-c6ccc(-c7ccccc7)cc6)c6c4CCCC6)CCCC5)cc3)cc2)cc1 ; CC1(C)c2ccccc2-c2ccc(N(c3ccc(-c4ccccc4)cc3)c3c4c(c(-c5ccc(-c6ccccc6)cc5)c5c3CCCC5)CCCC4)cc21]

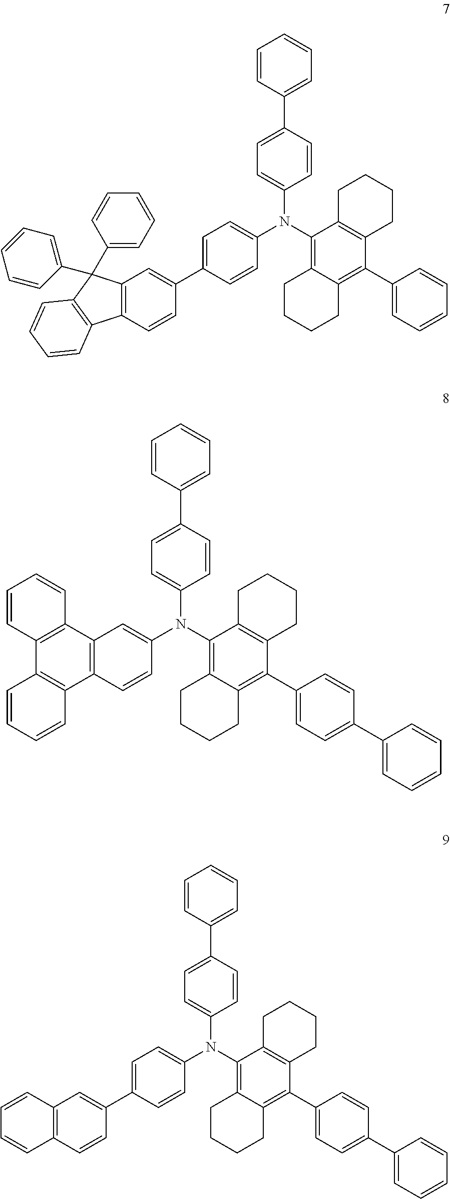 [SMILES: c1ccc(-c2ccc(N(c3ccc(-c4ccc5c(c4)C(c4ccccc4)(c4ccccc4)c4ccccc4-5)cc3)c3c4c(c(-c5ccccc5)c5c3CCCC5)CCCC4)cc2)cc1 ; c1ccc(-c2ccc(-c3c4c(c(N(c5ccc(-c6ccccc6)cc5)c5ccc6c7ccccc7c7ccccc7c6c5)c5c3CCCC5)CCCC4)cc2)cc1 ; c1ccc(-c2ccc(-c3c4c(c(N(c5ccc(-c6ccccc6)cc5)c5ccc(-c6ccc7ccccc7c6)cc5)c5c3CCCC5)CCCC4)cc2)cc1]

The material for an organic EL device may be represented by one or more of the following structures.

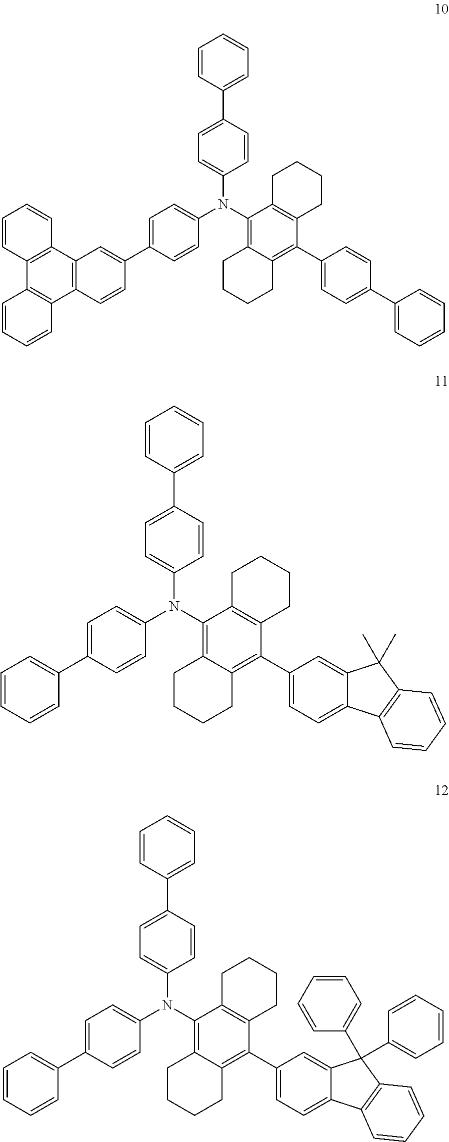 [SMILES: c1ccc(-c2ccc(-c3c4c(c(N(c5ccc(-c6ccccc6)cc5)c5ccc(-c6ccc7c8ccccc8c8ccccc8c7c6)cc5)c5c3CCCC5)CCCC4)cc2)cc1 ; CC1(C)c2ccccc2-c2ccc(-c3c4c(c(N(c5ccc(-c6ccccc6)cc5)c5ccc(-c6ccccc6)cc5)c5c3CCCC5)CCCC4)cc21 ; c1ccc(-c2ccc(N(c3ccc(-c4ccccc4)cc3)c3c4c(c(-c5ccc6c(c5)C(c5ccccc5)(c5ccccc5)c5ccccc5-6)c5c3CCCC5)CCCC4)cc2)cc1]

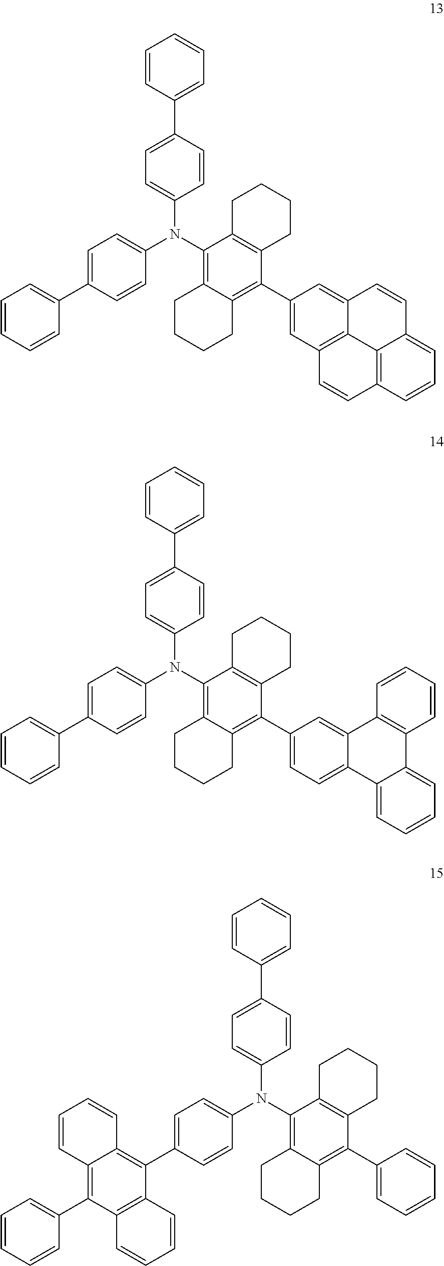 [SMILES: c1ccc(-c2ccc(N(c3ccc(-c4ccccc4)cc3)c3c4c(c(-c5cc6ccc7cccc8ccc(c5)c6c78)c5c3CCCC5)CCCC4)cc2)cc1 ; c1ccc(-c2ccc(N(c3ccc(-c4ccccc4)cc3)c3c4c(c(-c5ccc6c7ccccc7c7ccccc7c6c5)c5c3CCCC5)CCCC4)cc2)cc1 ; c1ccc(-c2ccc(N(c3ccc(-c4c5ccccc5c(-c5ccccc5)c5ccccc45)cc3)c3c4c(c(-c5ccccc5)c5c3CCCC5)CCCC4)cc2)cc1]

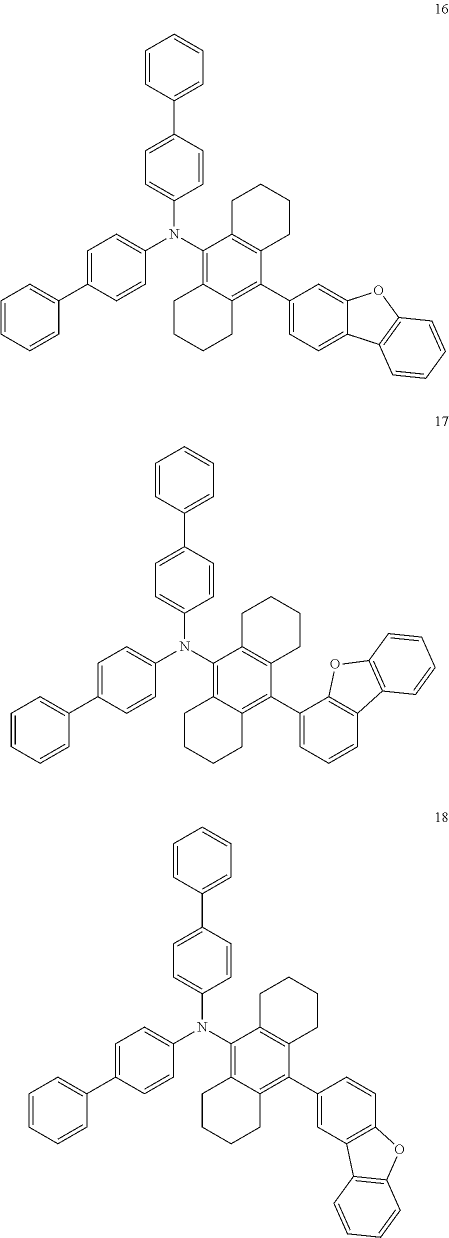 [SMILES: c1ccc(-c2ccc(N(c3ccc(-c4ccccc4)cc3)c3c4c(c(-c5ccc6c(c5)oc5ccccc56)c5c3CCCC5)CCCC4)cc2)cc1 ; c1ccc(-c2ccc(N(c3ccc(-c4ccccc4)cc3)c3c4c(c(-c5cccc6c5oc5ccccc56)c5c3CCCC5)CCCC4)cc2)cc1 ; c1ccc(-c2ccc(N(c3ccc(-c4ccccc4)cc3)c3c4c(c(-c5ccc6oc7ccccc7c6c5)c5c3CCCC5)CCCC4)cc2)cc1]

The material for an organic EL device may be represented by one or more of the following structures.

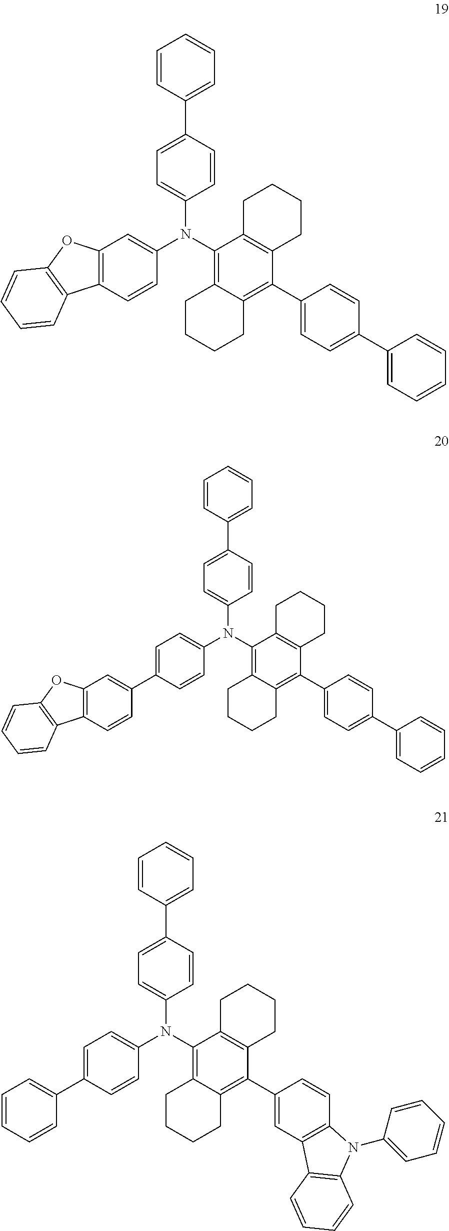 [SMILES: c1ccc(-c2ccc(-c3c4c(c(N(c5ccc(-c6ccccc6)cc5)c5ccc6c(c5)oc5ccccc56)c5c3CCCC5)CCCC4)cc2)cc1 ; c1ccc(-c2ccc(-c3c4c(c(N(c5ccc(-c6ccccc6)cc5)c5ccc(-c6ccc7c(c6)oc6ccccc67)cc5)c5c3CCCC5)CCCC4)cc2)cc1 ; c1ccc(-c2ccc(N(c3ccc(-c4ccccc4)cc3)c3c4c(c(-c5ccc6c(c5)c5ccccc5n6-c5ccccc5)c5c3CCCC5)CCCC4)cc2)cc1]

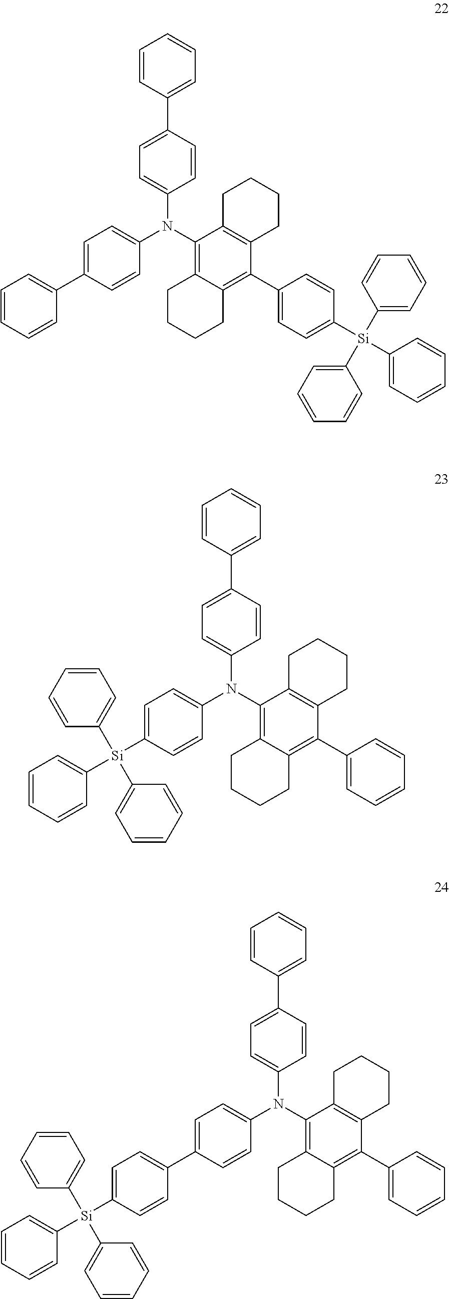 [SMILES: c1ccc(-c2ccc(N(c3ccc(-c4ccccc4)cc3)c3c4c(c(-c5ccc([Si](c6ccccc6)(c6ccccc6)c6ccccc6)cc5)c5c3CCCC5)CCCC4)cc2)cc1 ; c1ccc(-c2ccc(N(c3ccc([Si](c4ccccc4)(c4ccccc4)c4ccccc4)cc3)c3c4c(c(-c5ccccc5)c5c3CCCC5)CCCC4)cc2)cc1 ; c1ccc(-c2ccc(N(c3ccc(-c4ccc([Si](c5ccccc5)(c5ccccc5)c5ccccc5)cc4)cc3)c3c4c(c(-c5ccccc5)c5c3CCCC5)CCCC4)cc2)cc1]

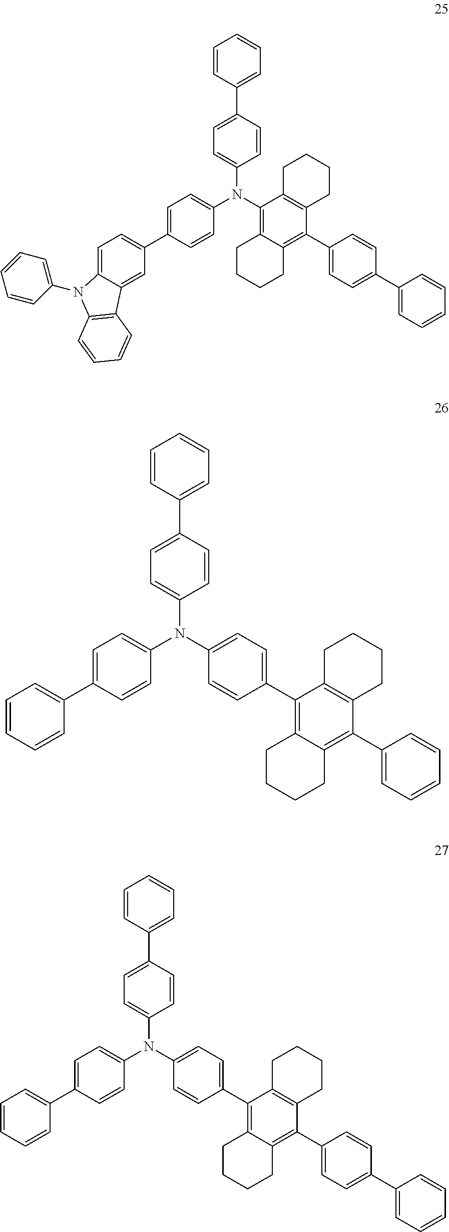 [SMILES: c1ccc(-c2ccc(-c3c4c(c(N(c5ccc(-c6ccccc6)cc5)c5ccc(-c6ccc7c(c6)c6ccccc6n7-c6ccccc6)cc5)c5c3CCCC5)CCCC4)cc2)cc1 ; c1ccc(-c2ccc(N(c3ccc(-c4ccccc4)cc3)c3ccc(-c4c5c(c(-c6ccccc6)c6c4CCCC6)CCCC5)cc3)cc2)cc1 ; c1ccc(-c2ccc(-c3c4c(c(-c5ccc(N(c6ccc(-c7ccccc7)cc6)c6ccc(-c7ccccc7)cc6)cc5)c5c3CCCC5)CCCC4)cc2)cc1]

The material for an organic EL device may be represented by one or more of the following structures.

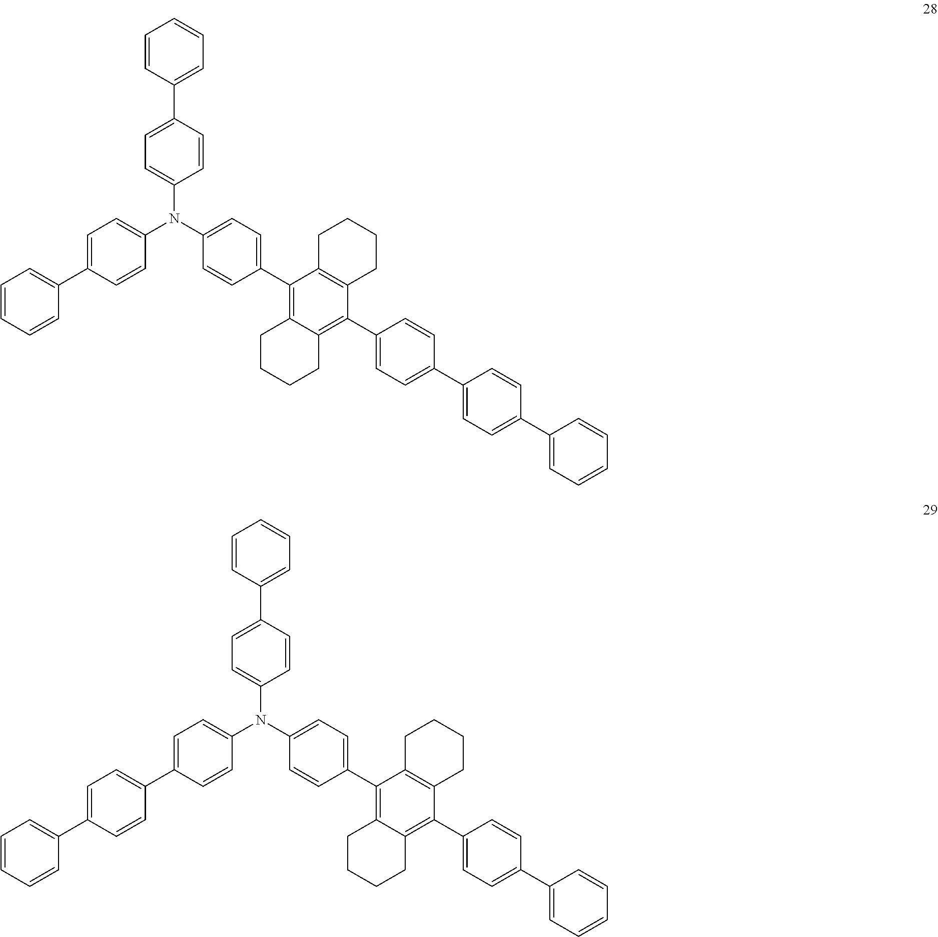 [SMILES: c1ccc(-c2ccc(-c3ccc(-c4c5c(c(-c6ccc(N(c7ccc(-c8ccccc8)cc7)c7ccc(-c8ccccc8)cc7)cc6)c6c4CCCC6)CCCC5)cc3)cc2)cc1 ; c1ccc(-c2ccc(-c3ccc(N(c4ccc(-c5ccccc5)cc4)c4ccc(-c5c6c(c(-c7ccc(-c8ccccc8)cc7)c7c5CCCC7)CCCC6)cc4)cc3)cc2)cc1]

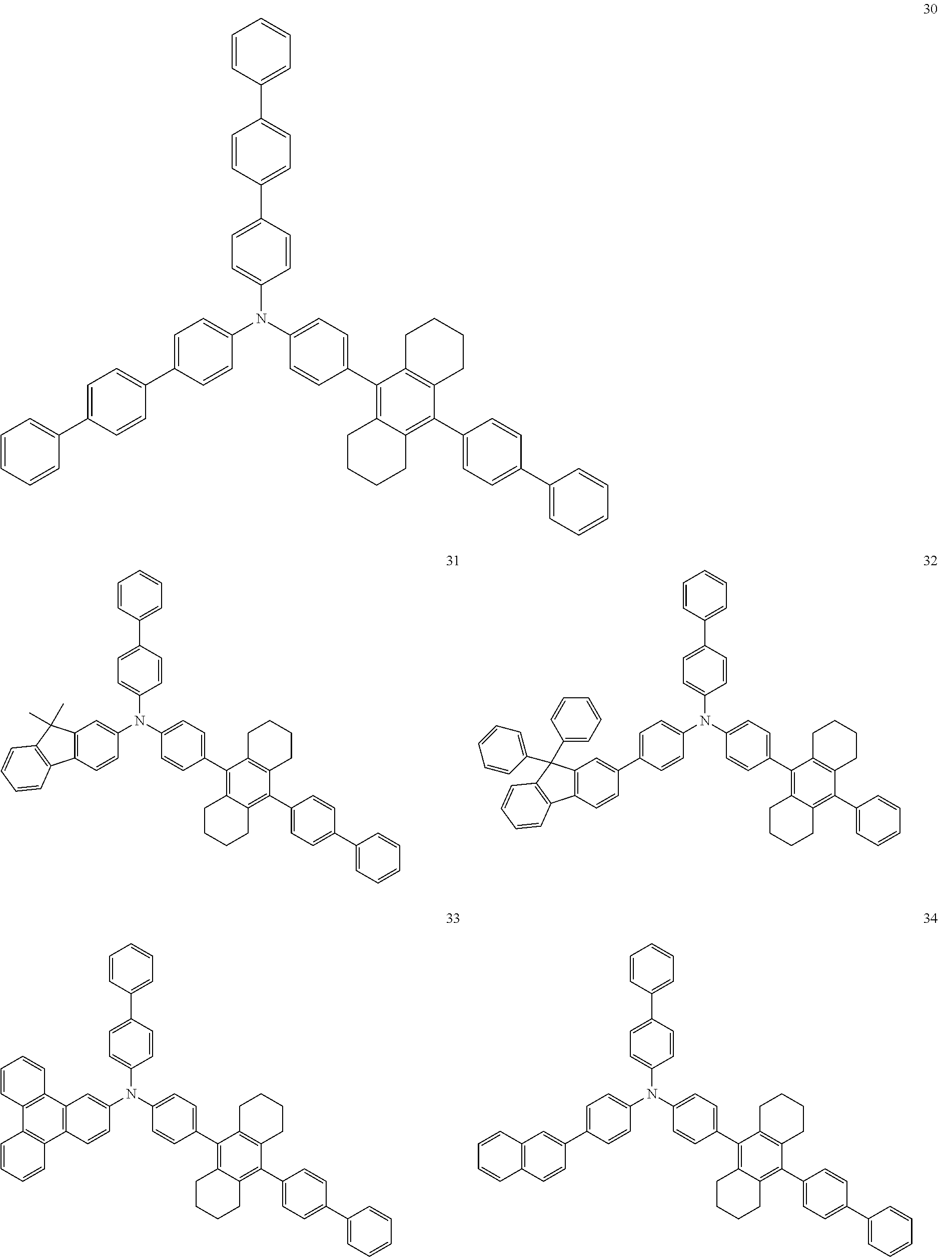

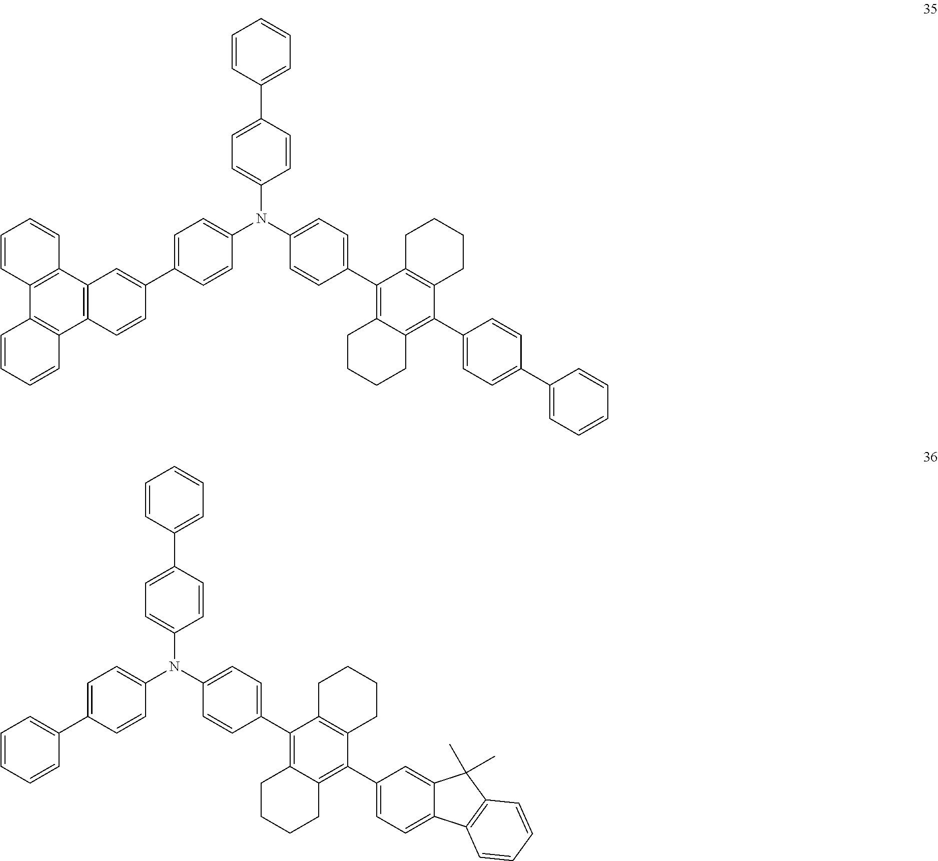 [SMILES: c1ccc(-c2ccc(-c3c4c(c(-c5ccc(N(c6ccc(-c7ccccc7)cc6)c6ccc(-c7ccc8c9ccccc9c9ccccc9c8c7)cc6)cc5)c5c3CCCC5)CCCC4)cc2)cc1 ; CC1(C)c2ccccc2-c2ccc(-c3c4c(c(-c5ccc(N(c6ccc(-c7ccccc7)cc6)c6ccc(-c7ccccc7)cc6)cc5)c5c3CCCC5)CCCC4)cc21]

The material for an organic EL device may be represented by one or more of the following structures.

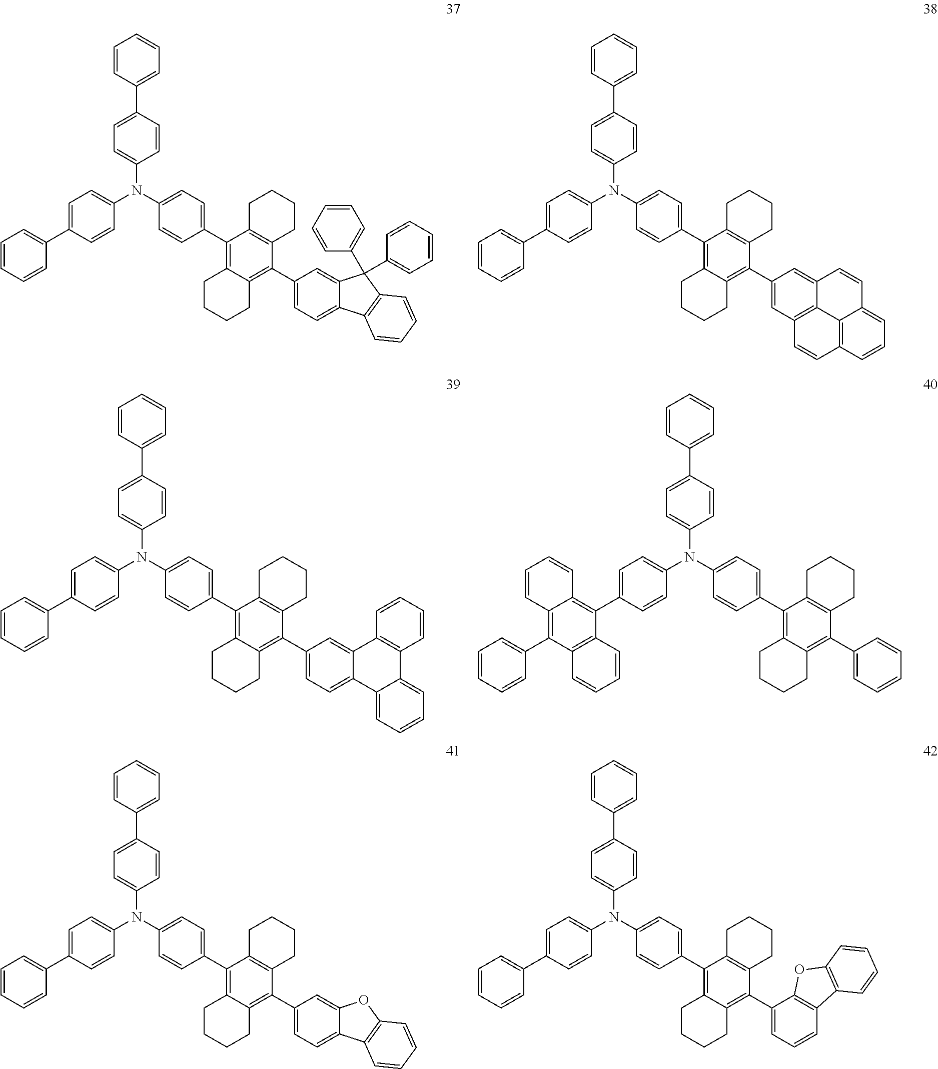

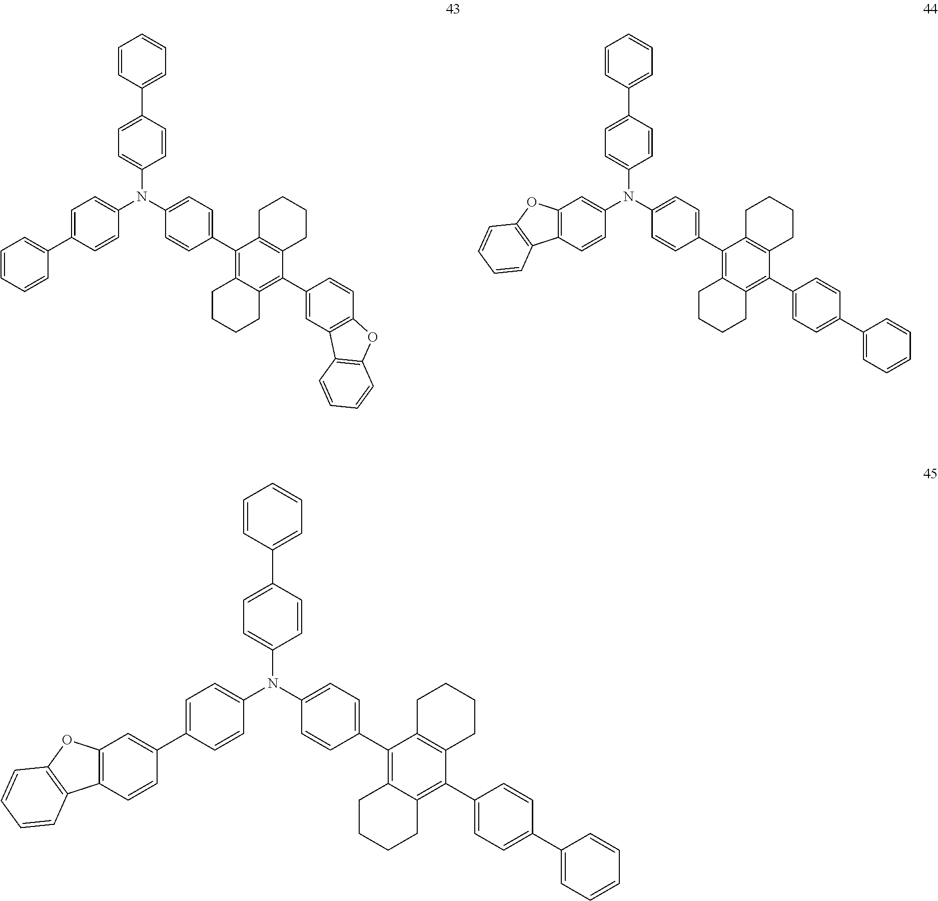 [SMILES: c1ccc(-c2ccc(N(c3ccc(-c4ccccc4)cc3)c3ccc(-c4c5c(c(-c6ccc7oc8ccccc8c7c6)c6c4CCCC6)CCCC5)cc3)cc2)cc1 ; c1ccc(-c2ccc(-c3c4c(c(-c5ccc(N(c6ccc(-c7ccccc7)cc6)c6ccc7c(c6)oc6ccccc67)cc5)c5c3CCCC5)CCCC4)cc2)cc1 ; c1ccc(-c2ccc(-c3c4c(c(-c5ccc(N(c6ccc(-c7ccccc7)cc6)c6ccc(-c7ccc8c(c7)oc7ccccc78)cc6)cc5)c5c3CCCC5)CCCC4)cc2)cc1]

The material for an organic EL device may be represented by one or more of the following structures.

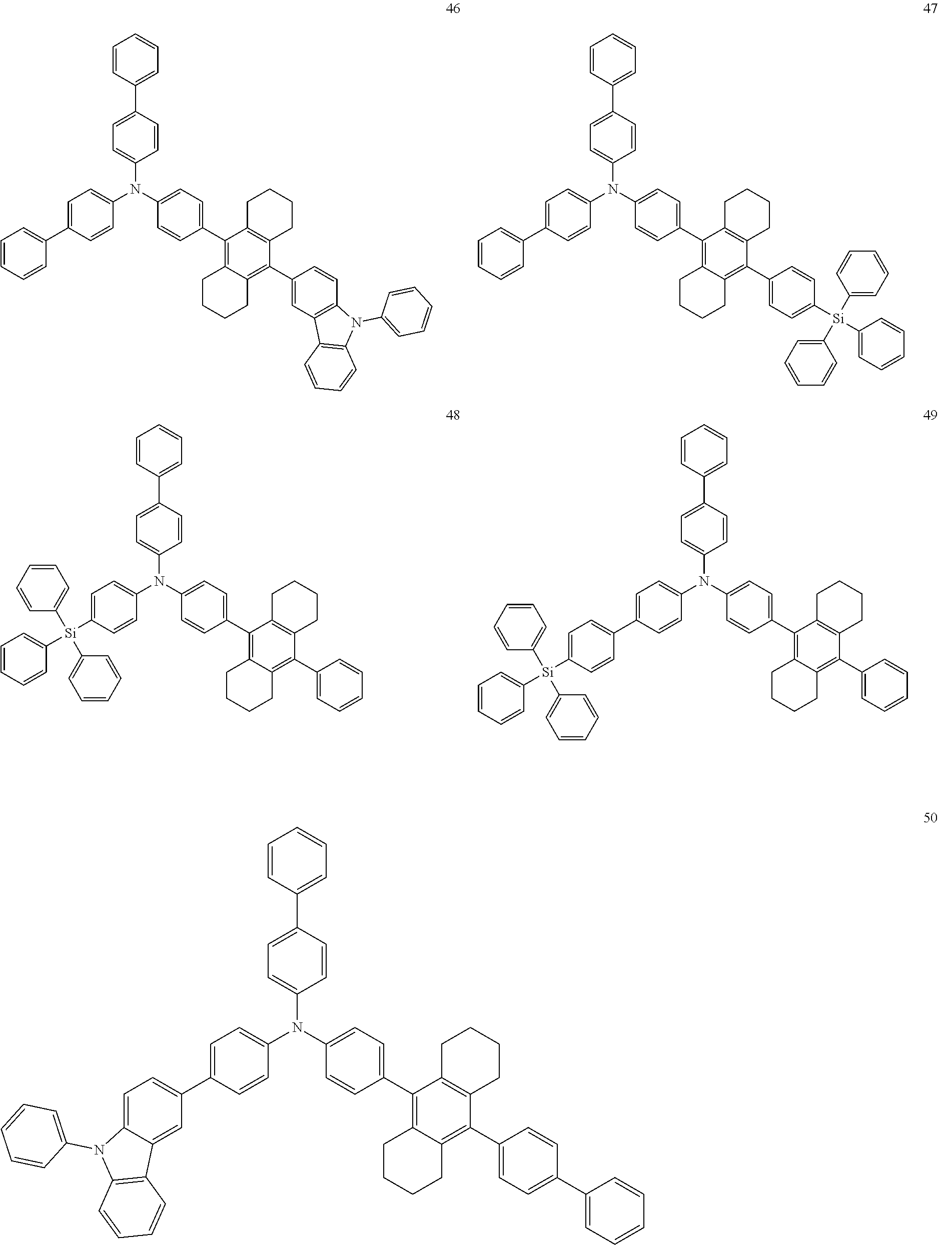

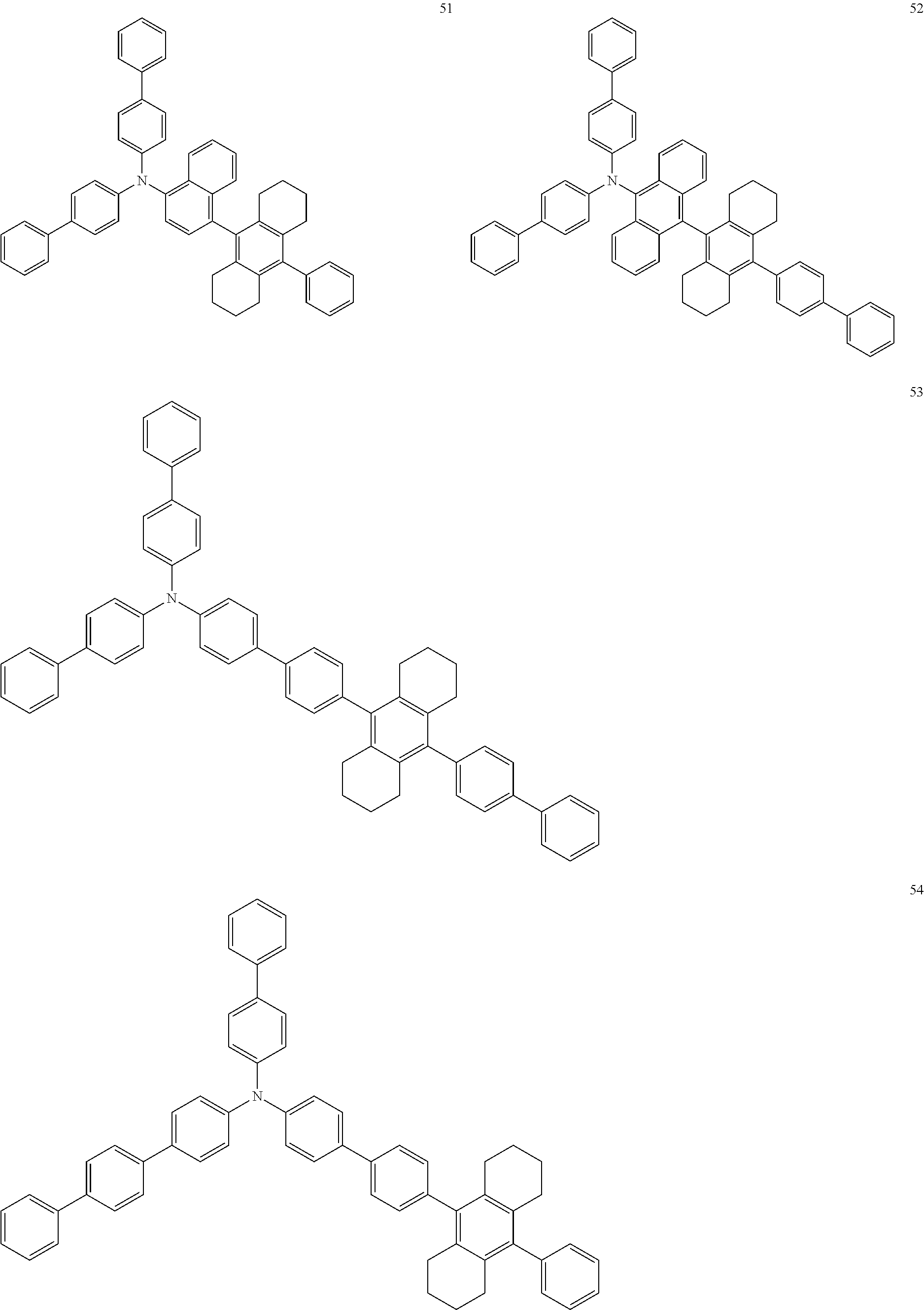 [SMILES: c1ccc(-c2ccc(N(c3ccc(-c4ccccc4)cc3)c3ccc(-c4c5c(c(-c6ccccc6)c6c4CCCC6)CCCC5)c4ccccc34)cc2)cc1 ; c1ccc(-c2ccc(-c3c4c(c(-c5c6ccccc6c(N(c6ccc(-c7ccccc7)cc6)c6ccc(-c7ccccc7)cc6)c6ccccc56)c5c3CCCC5)CCCC4)cc2)cc1 ; c1ccc(-c2ccc(-c3c4c(c(-c5ccc(-c6ccc(N(c7ccc(-c8ccccc8)cc7)c7ccc(-c8ccccc8)cc7)cc6)cc5)c5c3CCCC5)CCCC4)cc2)cc1 ; c1ccc(-c2ccc(-c3ccc(N(c4ccc(-c5ccccc5)cc4)c4ccc(-c5ccc(-c6c7c(c(-c8ccccc8)c8c6CCCC8)CCCC7)cc5)cc4)cc3)cc2)cc1]

The material for an organic EL device may be represented by one or more of the following structures.

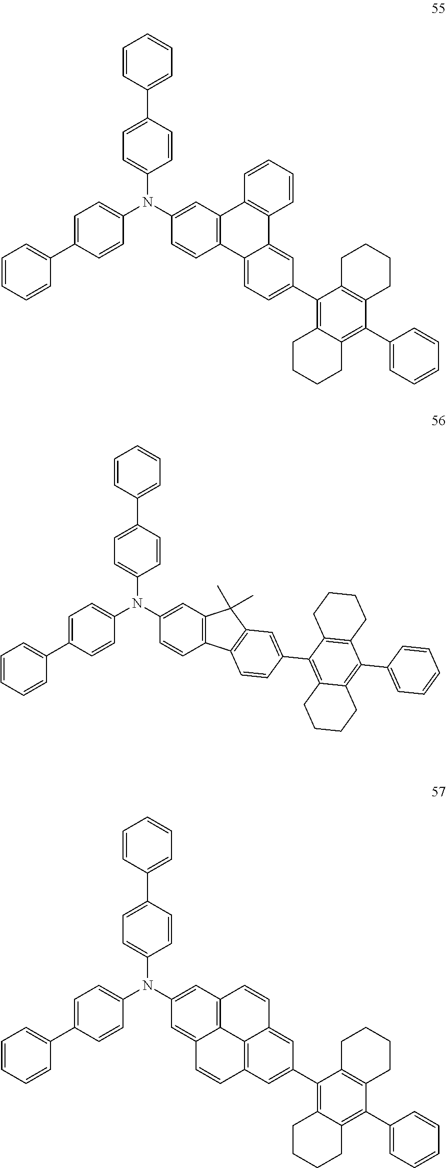 [SMILES: c1ccc(-c2ccc(N(c3ccc(-c4ccccc4)cc3)c3ccc4c5ccc(-c6c7c(c(-c8ccccc8)c8c6CCCC8)CCCC7)cc5c5ccccc5c4c3)cc2)cc1 ; CC1(C)c2cc(-c3c4c(c(-c5ccccc5)c5c3CCCC5)CCCC4)ccc2-c2ccc(N(c3ccc(-c4ccccc4)cc3)c3ccc(-c4ccccc4)cc3)cc21 ; c1ccc(-c2ccc(N(c3ccc(-c4ccccc4)cc3)c3cc4ccc5cc(-c6c7c(c(-c8ccccc8)c8c6CCCC8)CCCC7)cc6ccc(c3)c4c56)cc2)cc1]

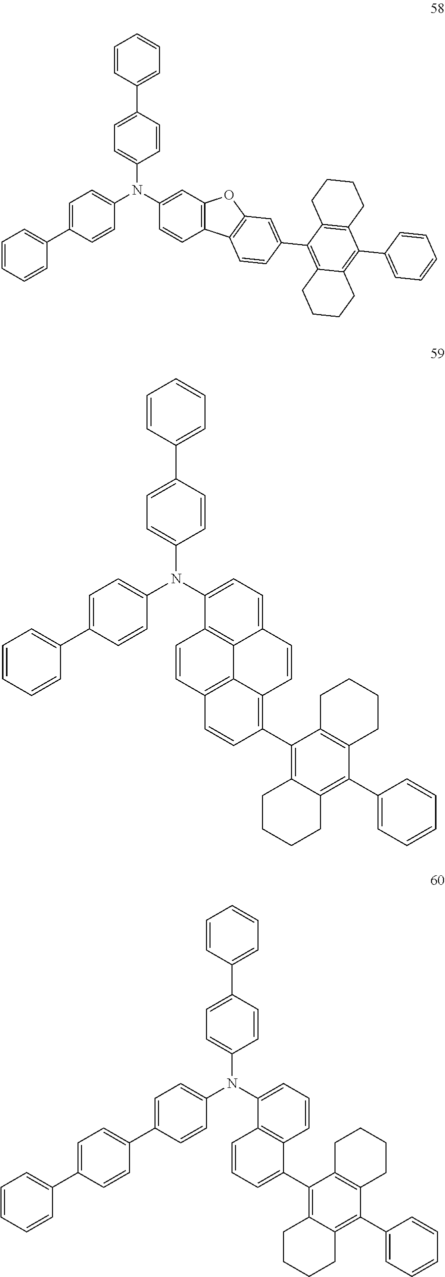 [SMILES: c1ccc(-c2ccc(N(c3ccc(-c4ccccc4)cc3)c3ccc4c(c3)oc3cc(-c5c6c(c(-c7ccccc7)c7c5CCCC7)CCCC6)ccc34)cc2)cc1 ; c1ccc(-c2ccc(N(c3ccc(-c4ccccc4)cc3)c3ccc4ccc5c(-c6c7c(c(-c8ccccc8)c8c6CCCC8)CCCC7)ccc6ccc3c4c65)cc2)cc1 ; c1ccc(-c2ccc(-c3ccc(N(c4ccc(-c5ccccc5)cc4)c4cccc5c(-c6c7c(c(-c8ccccc8)c8c6CCCC8)CCCC7)cccc45)cc3)cc2)cc1]

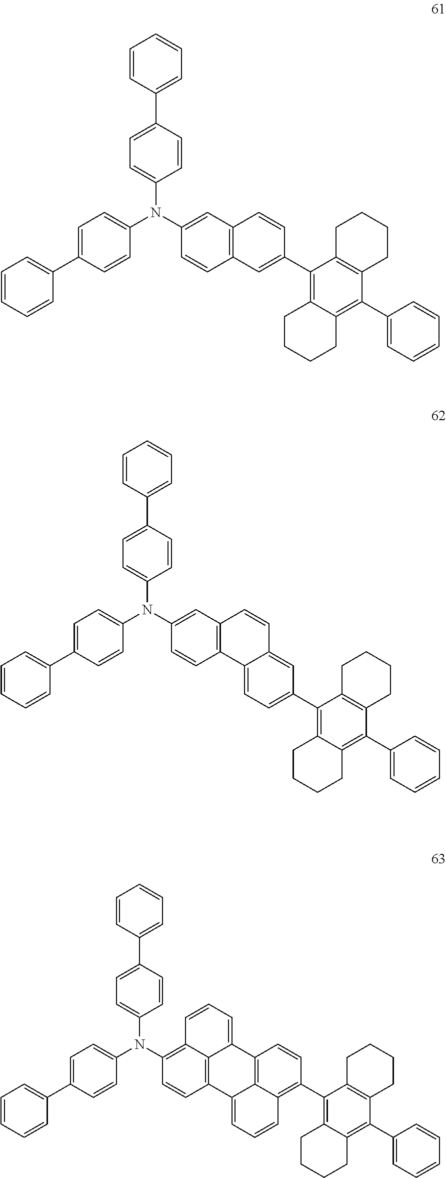 [SMILES: c1ccc(-c2ccc(N(c3ccc(-c4ccccc4)cc3)c3ccc4cc(-c5c6c(c(-c7ccccc7)c7c5CCCC7)CCCC6)ccc4c3)cc2)cc1 ; c1ccc(-c2ccc(N(c3ccc(-c4ccccc4)cc3)c3ccc4c(ccc5cc(-c6c7c(c(-c8ccccc8)c8c6CCCC8)CCCC7)ccc54)c3)cc2)cc1 ; c1ccc(-c2ccc(N(c3ccc(-c4ccccc4)cc3)c3ccc4c5cccc6c(-c7c8c(c(-c9ccccc9)c9c7CCCC9)CCCC8)ccc(c7cccc3c74)c65)cc2)cc1]

The material for an organic EL device may be represented by one or more of the following structures.

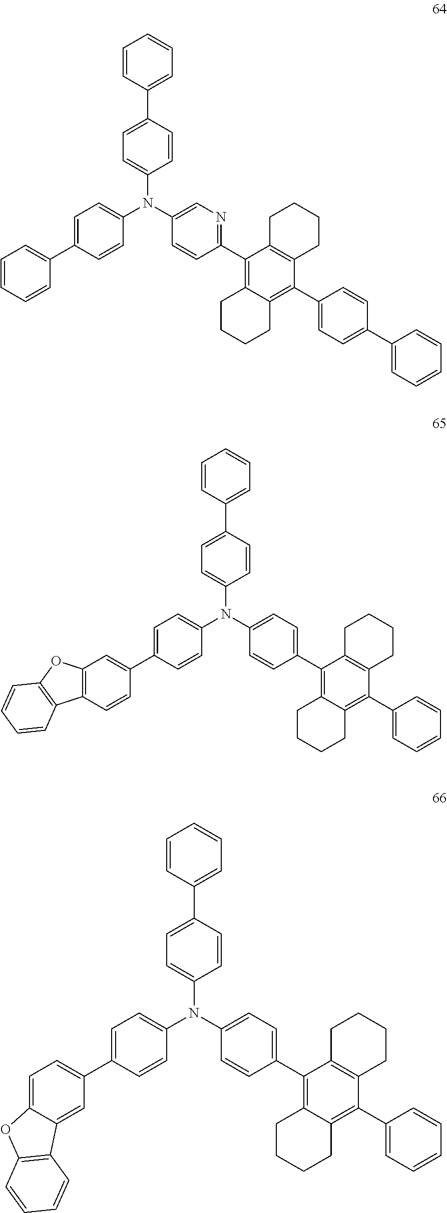 [SMILES: c1ccc(-c2ccc(-c3c4c(c(-c5ccc(N(c6ccc(-c7ccccc7)cc6)c6ccc(-c7ccccc7)cc6)cn5)c5c3CCCC5)CCCC4)cc2)cc1 ; c1ccc(-c2ccc(N(c3ccc(-c4ccc5c(c4)oc4ccccc45)cc3)c3ccc(-c4c5c(c(-c6ccccc6)c6c4CCCC6)CCCC5)cc3)cc2)cc1 ; c1ccc(-c2ccc(N(c3ccc(-c4ccc5oc6ccccc6c5c4)cc3)c3ccc(-c4c5c(c(-c6ccccc6)c6c4CCCC6)CCCC5)cc3)cc2)cc1]

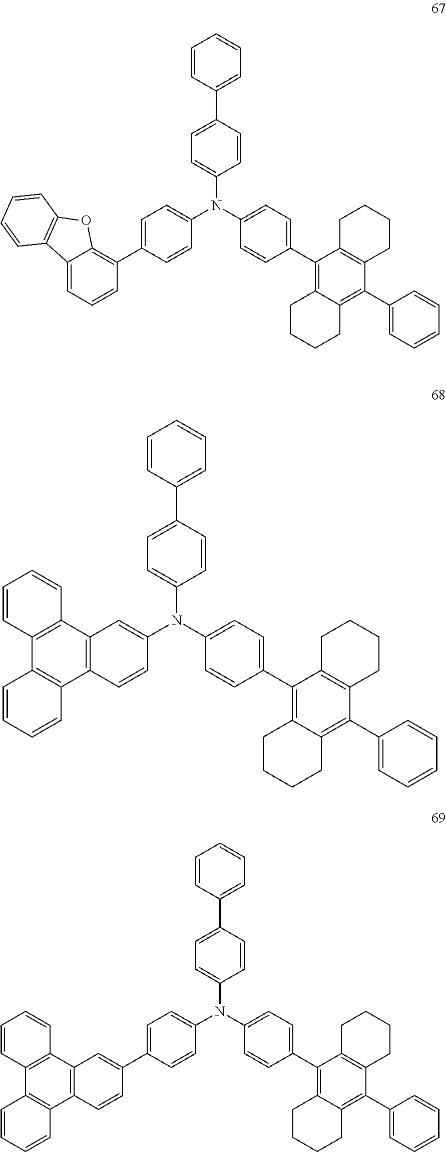 [SMILES: c1ccc(-c2ccc(N(c3ccc(-c4c5c(c(-c6ccccc6)c6c4CCCC6)CCCC5)cc3)c3ccc(-c4cccc5c4oc4ccccc45)cc3)cc2)cc1 ; c1ccc(-c2ccc(N(c3ccc(-c4c5c(c(-c6ccccc6)c6c4CCCC6)CCCC5)cc3)c3ccc4c5ccccc5c5ccccc5c4c3)cc2)cc1 ; c1ccc(-c2ccc(N(c3ccc(-c4ccc5c6ccccc6c6ccccc6c5c4)cc3)c3ccc(-c4c5c(c(-c6ccccc6)c6c4CCCC6)CCCC5)cc3)cc2)cc1]

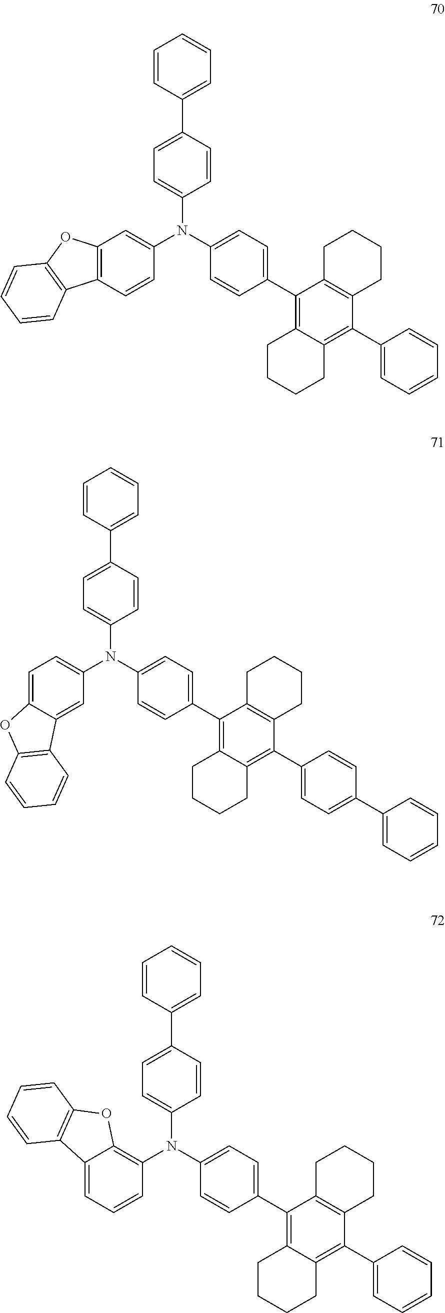 [SMILES: c1ccc(-c2ccc(N(c3ccc(-c4c5c(c(-c6ccccc6)c6c4CCCC6)CCCC5)cc3)c3ccc4c(c3)oc3ccccc34)cc2)cc1 ; c1ccc(-c2ccc(-c3c4c(c(-c5ccc(N(c6ccc(-c7ccccc7)cc6)c6ccc7oc8ccccc8c7c6)cc5)c5c3CCCC5)CCCC4)cc2)cc1 ; c1ccc(-c2ccc(N(c3ccc(-c4c5c(c(-c6ccccc6)c6c4CCCC6)CCCC5)cc3)c3cccc4c3oc3ccccc34)cc2)cc1]

The material for an organic EL device may be represented by one or more of the following structures.

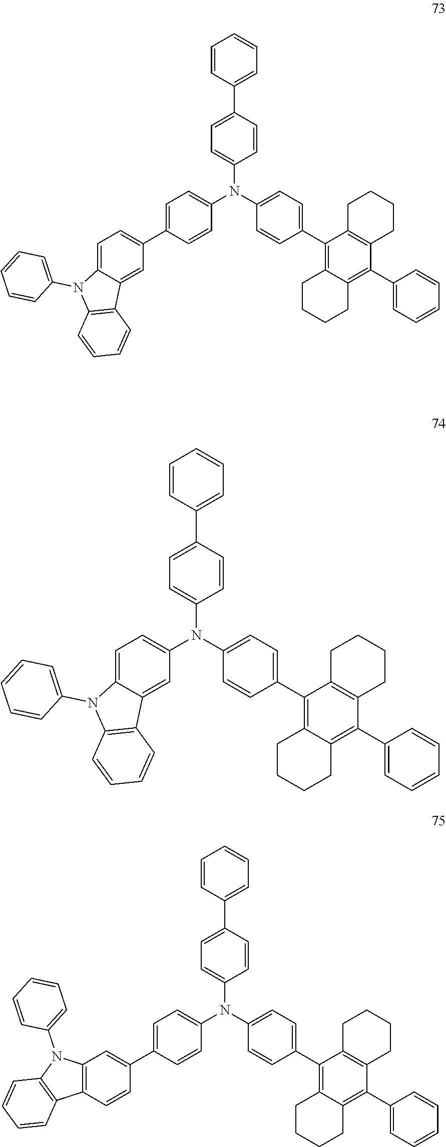 [SMILES: c1ccc(-c2ccc(N(c3ccc(-c4ccc5c(c4)c4ccccc4n5-c4ccccc4)cc3)c3ccc(-c4c5c(c(-c6ccccc6)c6c4CCCC6)CCCC5)cc3)cc2)cc1 ; c1ccc(-c2ccc(N(c3ccc(-c4c5c(c(-c6ccccc6)c6c4CCCC6)CCCC5)cc3)c3ccc4c(c3)c3ccccc3n4-c3ccccc3)cc2)cc1 ; c1ccc(-c2ccc(N(c3ccc(-c4ccc5c6ccccc6n(-c6ccccc6)c5c4)cc3)c3ccc(-c4c5c(c(-c6ccccc6)c6c4CCCC6)CCCC5)cc3)cc2)cc1]

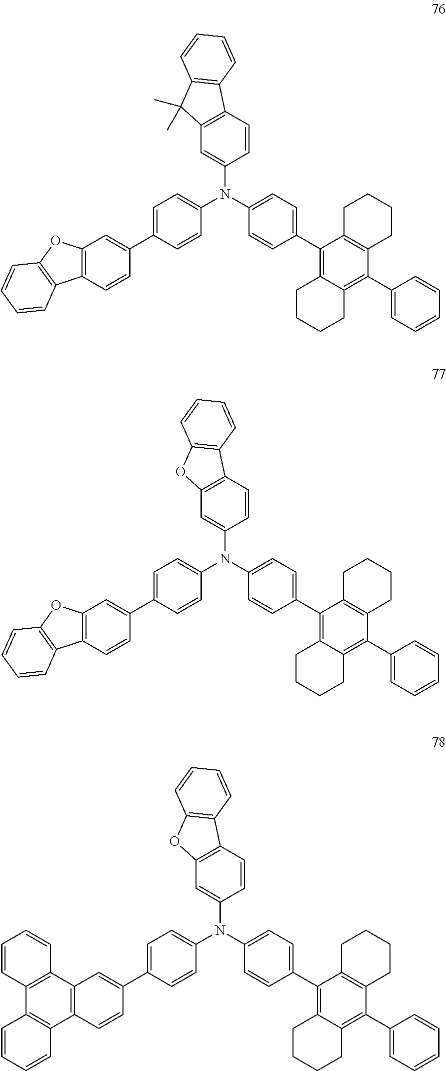 [SMILES: CC1(C)c2ccccc2-c2ccc(N(c3ccc(-c4ccc5c(c4)oc4ccccc45)cc3)c3ccc(-c4c5c(c(-c6ccccc6)c6c4CCCC6)CCCC5)cc3)cc21 ; c1ccc(-c2c3c(c(-c4ccc(N(c5ccc(-c6ccc7c(c6)oc6ccccc67)cc5)c5ccc6c(c5)oc5ccccc56)cc4)c4c2CCCC4)CCCC3)cc1 ; c1ccc(-c2c3c(c(-c4ccc(N(c5ccc(-c6ccc7c8ccccc8c8ccccc8c7c6)cc5)c5ccc6c(c5)oc5ccccc56)cc4)c4c2CCCC4)CCCC3)cc1]

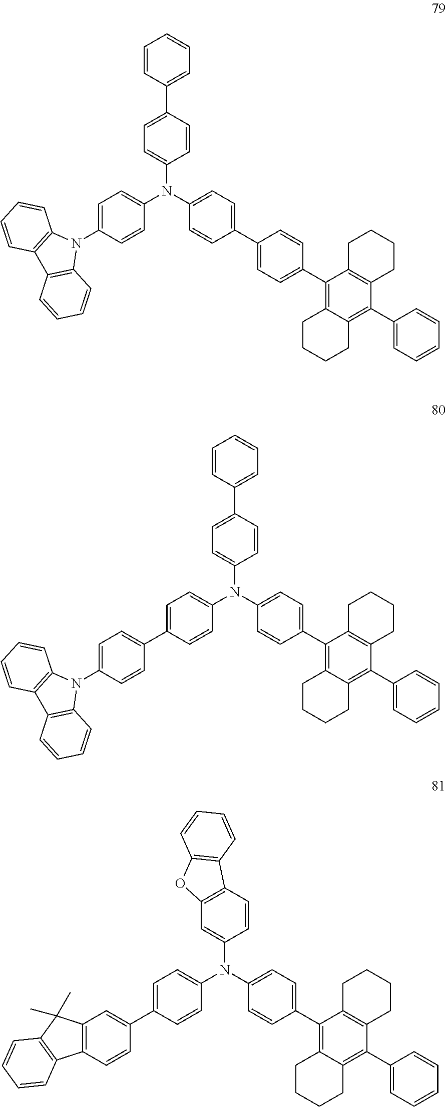 [SMILES: c1ccc(-c2ccc(N(c3ccc(-c4ccc(-c5c6c(c(-c7ccccc7)c7c5CCCC7)CCCC6)cc4)cc3)c3ccc(-n4c5ccccc5c5ccccc54)cc3)cc2)cc1 ; c1ccc(-c2ccc(N(c3ccc(-c4ccc(-n5c6ccccc6c6ccccc65)cc4)cc3)c3ccc(-c4c5c(c(-c6ccccc6)c6c4CCCC6)CCCC5)cc3)cc2)cc1 ; CC1(C)c2ccccc2-c2ccc(-c3ccc(N(c4ccc(-c5c6c(c(-c7ccccc7)c7c5CCCC7)CCCC6)cc4)c4ccc5c(c4)oc4ccccc45)cc3)cc21]

The material for an organic EL device may be represented by one or more of the following structures.

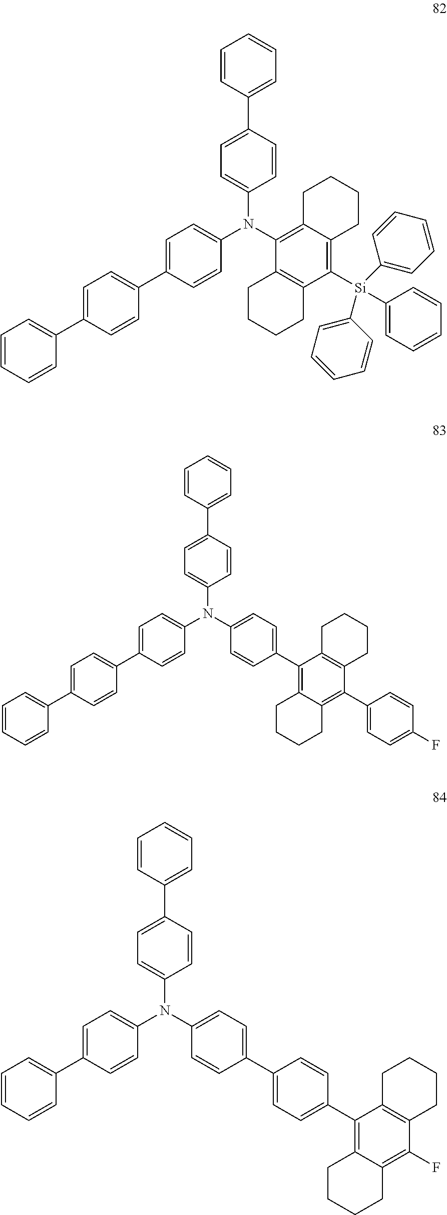 [SMILES: c1ccc(-c2ccc(-c3ccc(N(c4ccc(-c5ccccc5)cc4)c4c5c(c([Si](c6ccccc6)(c6ccccc6)c6ccccc6)c6c4CCCC6)CCCC5)cc3)cc2)cc1 ; Fc1ccc(-c2c3c(c(-c4ccc(N(c5ccc(-c6ccccc6)cc5)c5ccc(-c6ccc(-c7ccccc7)cc6)cc5)cc4)c4c2CCCC4)CCCC3)cc1 ; Fc1c2c(c(-c3ccc(-c4ccc(N(c5ccc(-c6ccccc6)cc5)c5ccc(-c6ccccc6)cc5)cc4)cc3)c3c1CCCC3)CCCC2]

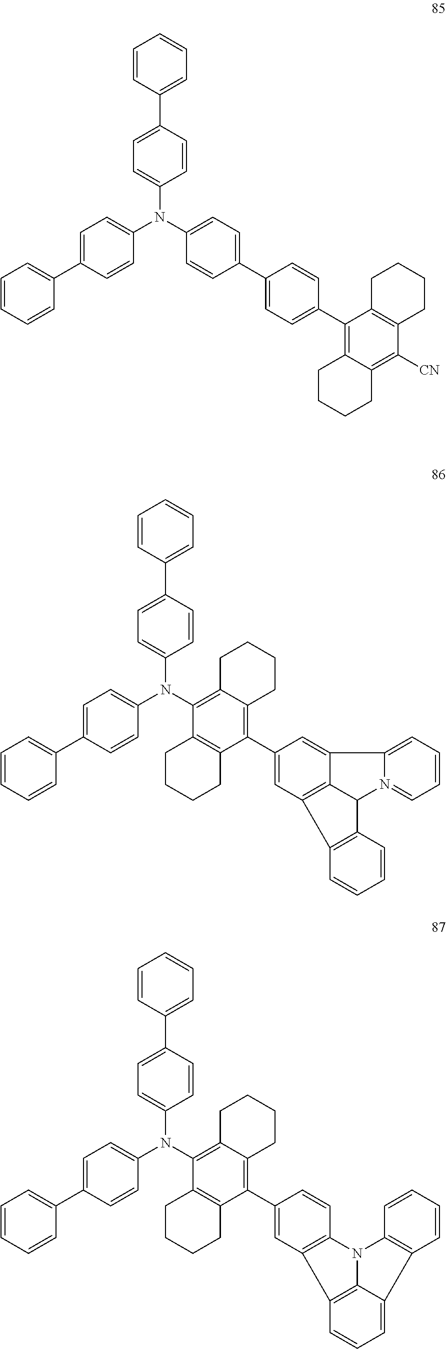

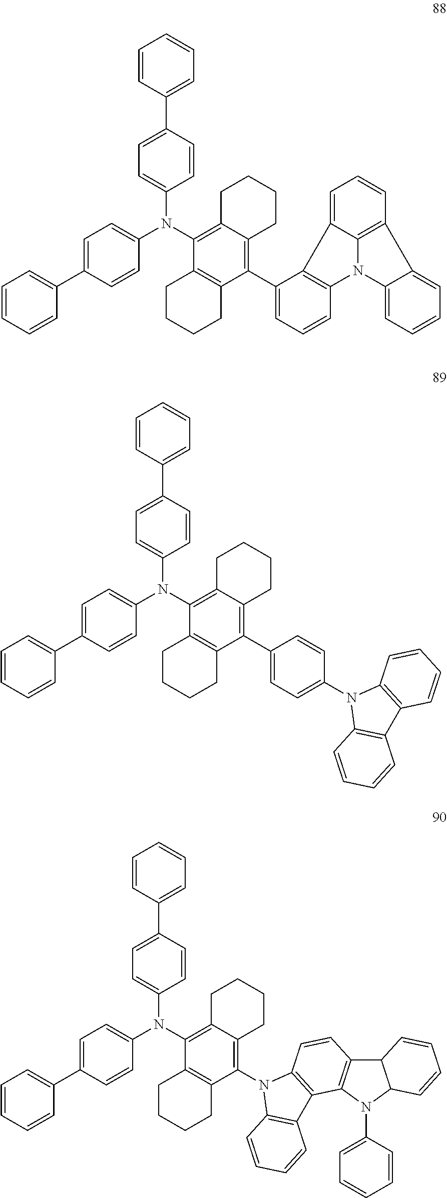 [SMILES: c1ccc(-c2ccc(N(c3ccc(-c4ccccc4)cc3)c3c4c(c(-c5cccc6c5c5cccc7c8ccccc8n6c75)c5c3CCCC5)CCCC4)cc2)cc1 ; c1ccc(-c2ccc(N(c3ccc(-c4ccccc4)cc3)c3c4c(c(-c5ccc(-n6c7ccccc7c7ccccc76)cc5)c5c3CCCC5)CCCC4)cc2)cc1 ; C1=CC2c3ccc4c(c3N(c3ccccc3)C2C=C1)c1ccccc1n4-c1c2c(c(N(c3ccc(-c4ccccc4)cc3)c3ccc(-c4ccccc4)cc3)c3c1CCCC3)CCCC2]

The material for an organic EL device may be represented by one or more of the following structures.

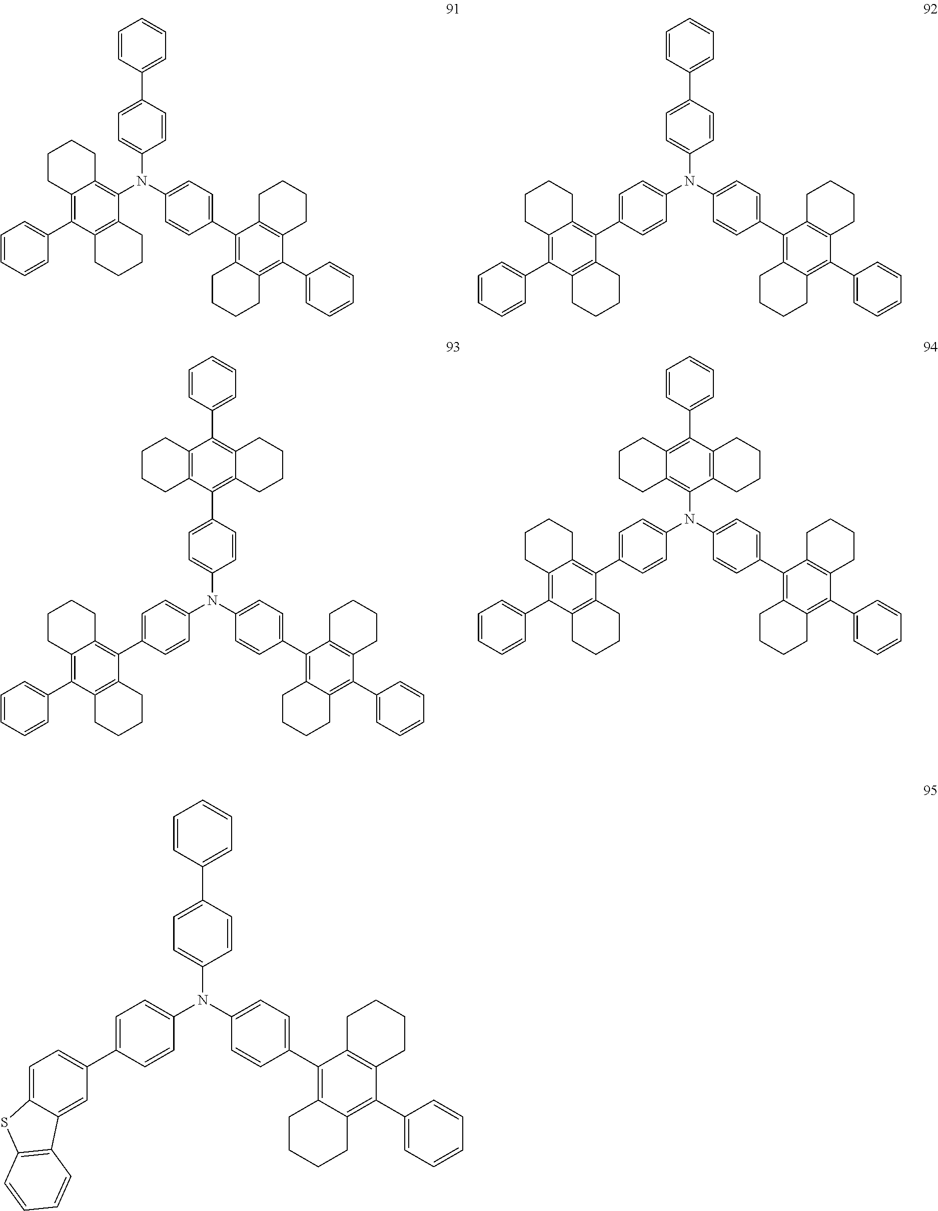

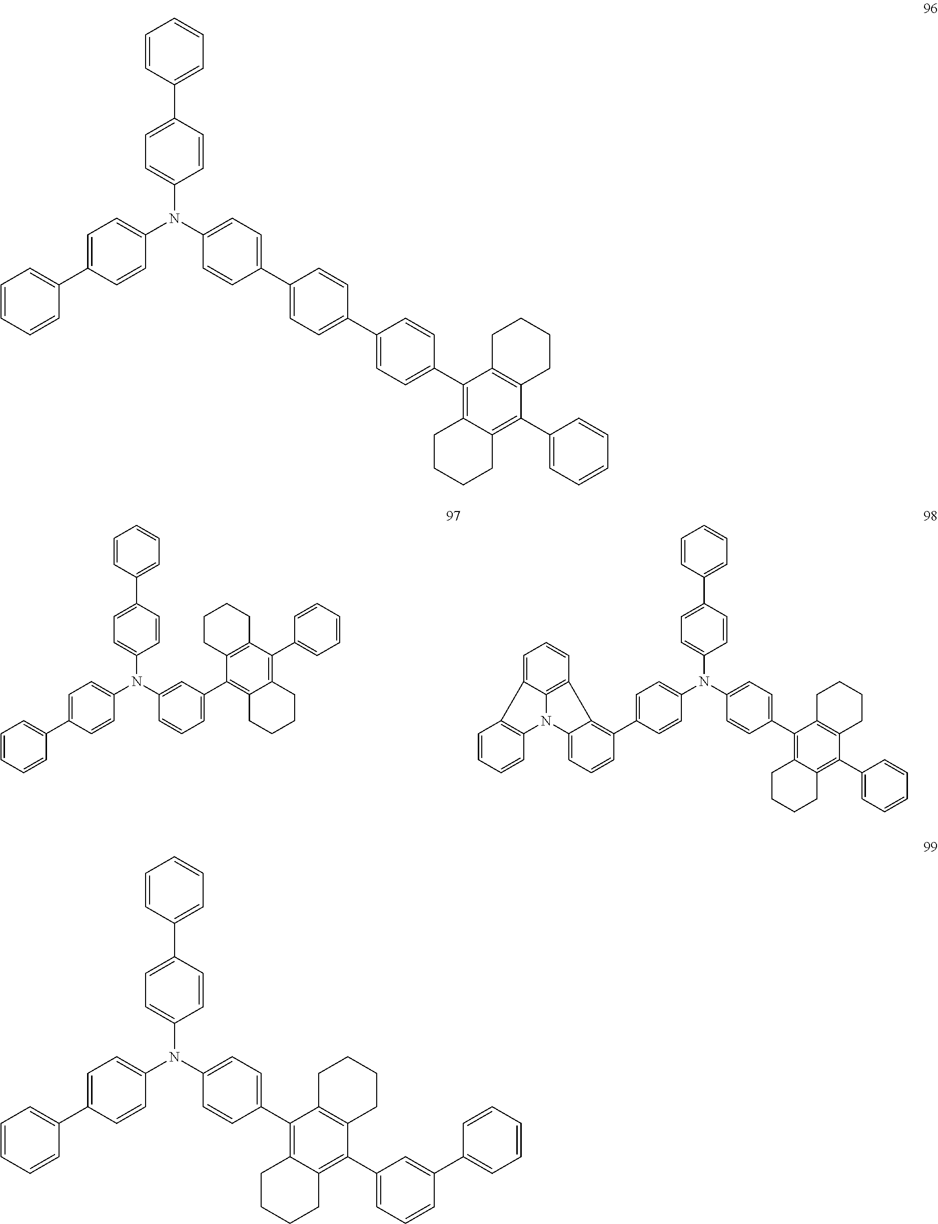 [SMILES: c1ccc(-c2ccc(N(c3ccc(-c4ccccc4)cc3)c3ccc(-c4ccc(-c5ccc(-c6c7c(c(-c8ccccc8)c8c6CCCC8)CCCC7)cc5)cc4)cc3)cc2)cc1 ; c1ccc(-c2ccc(N(c3ccc(-c4ccccc4)cc3)c3cccc(-c4c5c(c(-c6ccccc6)c6c4CCCC6)CCCC5)c3)cc2)cc1 ; c1ccc(-c2ccc(N(c3ccc(-c4c5c(c(-c6ccccc6)c6c4CCCC6)CCCC5)cc3)c3ccc(-c4cccc5c4c4cccc6c7ccccc7n5c64)cc3)cc2)cc1 ; c1ccc(-c2ccc(N(c3ccc(-c4ccccc4)cc3)c3ccc(-c4c5c(c(-c6cccc(-c7ccccc7)c6)c6c4CCCC6)CCCC5)cc3)cc2)cc1]

The material for an organic EL device may be represented by one or more of the following structures s.

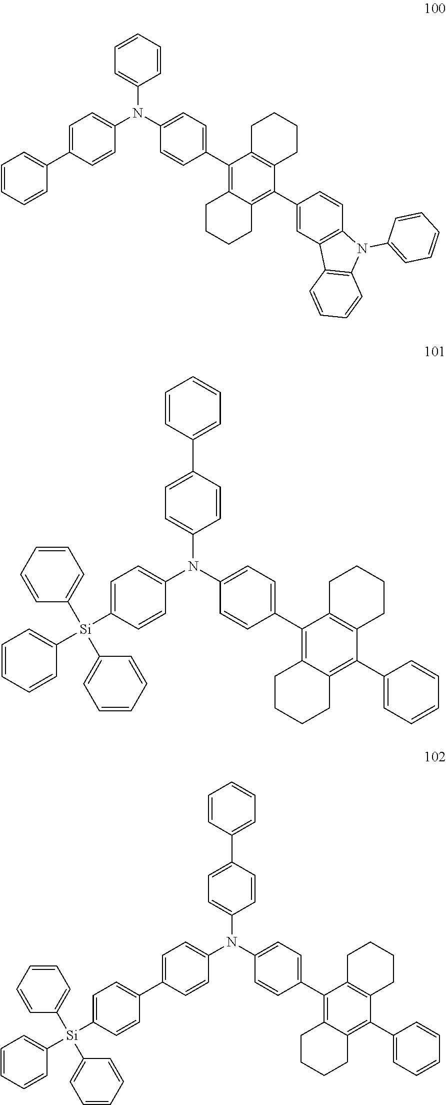 [SMILES: c1ccc(-c2ccc(N(c3ccccc3)c3ccc(-c4c5c(c(-c6ccc7c(c6)c6ccccc6n7-c6ccccc6)c6c4CCCC6)CCCC5)cc3)cc2)cc1 ; c1ccc(-c2ccc(N(c3ccc(-c4c5c(c(-c6ccccc6)c6c4CCCC6)CCCC5)cc3)c3ccc([Si](c4ccccc4)(c4ccccc4)c4ccccc4)cc3)cc2)cc1 ; c1ccc(-c2ccc(N(c3ccc(-c4ccc([Si](c5ccccc5)(c5ccccc5)c5ccccc5)cc4)cc3)c3ccc(-c4c5c(c(-c6ccccc6)c6c4CCCC6)CCCC5)cc3)cc2)cc1]

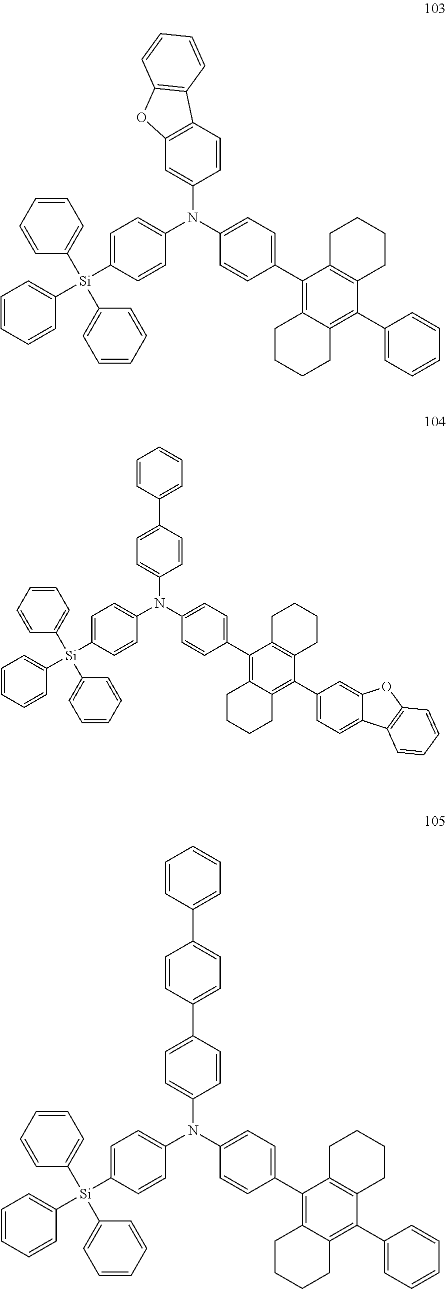 [SMILES: c1ccc(-c2c3c(c(-c4ccc(N(c5ccc([Si](c6ccccc6)(c6ccccc6)c6ccccc6)cc5)c5ccc6c(c5)oc5ccccc56)cc4)c4c2CCCC4)CCCC3)cc1 ; c1ccc(-c2ccc(N(c3ccc(-c4c5c(c(-c6ccc7c(c6)oc6ccccc67)c6c4CCCC6)CCCC5)cc3)c3ccc([Si](c4ccccc4)(c4ccccc4)c4ccccc4)cc3)cc2)cc1 ; c1ccc(-c2ccc(-c3ccc(N(c4ccc(-c5c6c(c(-c7ccccc7)c7c5CCCC7)CCCC6)cc4)c4ccc([Si](c5ccccc5)(c5ccccc5)c5ccccc5)cc4)cc3)cc2)cc1]

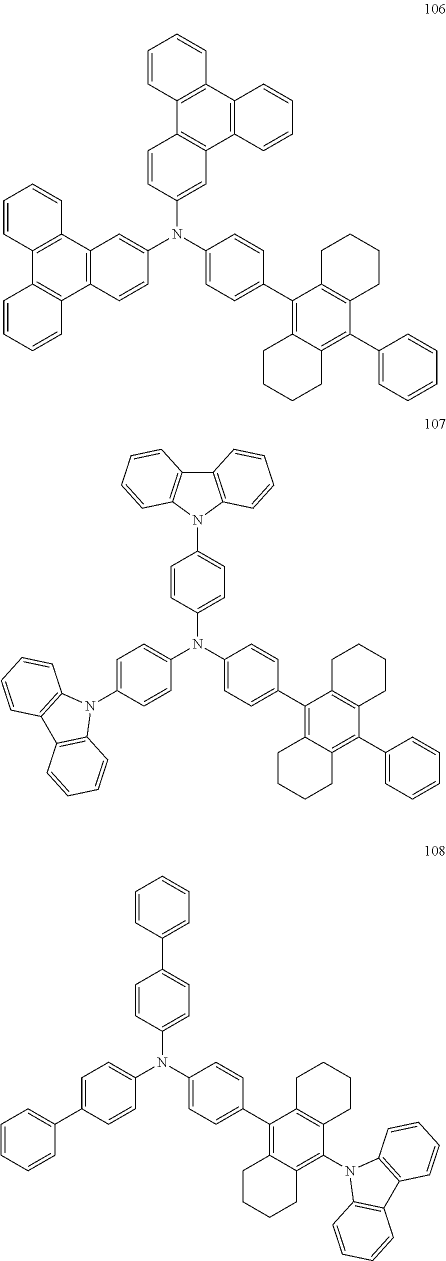 [SMILES: c1ccc(-c2c3c(c(-c4ccc(N(c5ccc6c7ccccc7c7ccccc7c6c5)c5ccc6c7ccccc7c7ccccc7c6c5)cc4)c4c2CCCC4)CCCC3)cc1 ; c1ccc(-c2c3c(c(-c4ccc(N(c5ccc(-n6c7ccccc7c7ccccc76)cc5)c5ccc(-n6c7ccccc7c7ccccc76)cc5)cc4)c4c2CCCC4)CCCC3)cc1 ; c1ccc(-c2ccc(N(c3ccc(-c4ccccc4)cc3)c3ccc(-c4c5c(c(-n6c7ccccc7c7ccccc76)c6c4CCCC6)CCCC5)cc3)cc2)cc1]

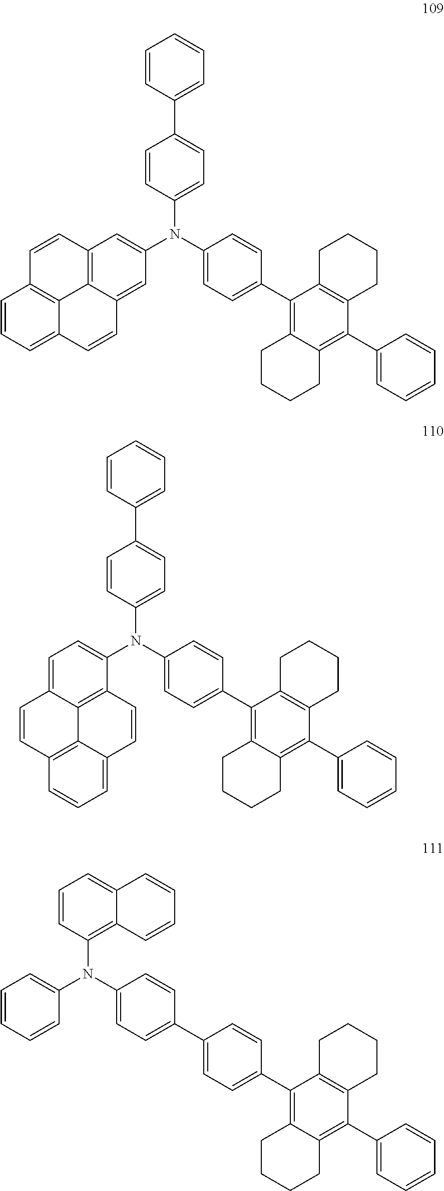 [SMILES: c1ccc(-c2ccc(N(c3ccc(-c4c5c(c(-c6ccccc6)c6c4CCCC6)CCCC5)cc3)c3cc4ccc5cccc6ccc(c3)c4c56)cc2)cc1 ; c1ccc(-c2ccc(N(c3ccc(-c4c5c(c(-c6ccccc6)c6c4CCCC6)CCCC5)cc3)c3ccc4ccc5cccc6ccc3c4c56)cc2)cc1 ; c1ccc(-c2c3c(c(-c4ccc(-c5ccc(N(c6ccccc6)c6cccc7ccccc67)cc5)cc4)c4c2CCCC4)CCCC3)cc1]

The material for an organic EL device may be used as a hole transport material for the organic EL device. Thus, by using the material for an organic EL device in a layer of stacked layers disposed between an emission layer and an anode, an organic EL device driven at a low voltage and having high emission efficiency may be manufactured.

(Organic EL Device)

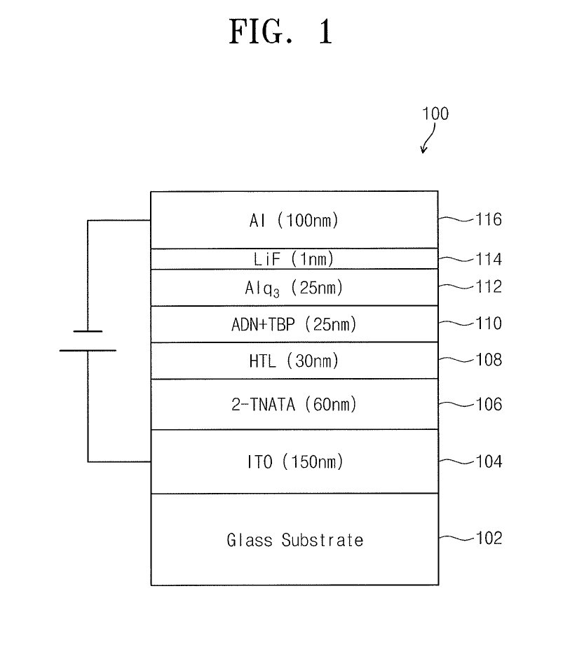

An organic EL device using the material for an organic EL device according to embodiments will be explained. FIG. 1 illustrates a schematic diagram depicting a configuration of an organic EL device 100 according to an embodiment. The organic EL device 100 may include, for example, a substrate 102, an anode 104, a hole injection layer 106, a hole transport layer 108, an emission layer 110, an electron transport layer 112, an electron injection layer 114, and a cathode 116. (FIG. 1 further provides, as an example, the particular materials used in the organic EL devices of the Examples, below.) In an embodiment, the material for an organic EL device according to embodiments may be used in the emission layer 110 of the organic EL device.

For example, an embodiment using the material for an organic EL device in the hole transport layer 108 will be explained. The substrate 102 may be a transparent glass substrate. In other implementations, the substrate 102 may be a semiconductor substrate formed by using silicon, etc., or a flexible substrate of a resin, etc. The anode 104 may be disposed on the substrate 102. The anode 104 may be formed using indium tin oxide (ITO), indium zinc oxide (IZO), etc. The hole injection layer 106 may be disposed on the anode 104. The hole injection layer 106 may include, for example, 4,4′,4″-tris[2-naphthyl)(phenyl)amino]triphenylamine (2-TNATA), N,N,N′,N′-tetrakis(3-methylphenyl)-3,3′-dimethylbenzidine (HMTPD), dipyrazino[2,3-f:2′,3′-h]quinoxaline-2,3,6,7,10,11-hexacarbonitrle (HAT(CN)₆), etc. The hole transport layer 108 may be disposed on the hole injection layer 106. The hole transport layer 108 may be formed using the material for an organic EL device according to embodiments. The emission layer 110 may be disposed on the hole transport layer 108. The emission layer 110 may be formed using, for example, a host material including 9,10-di(2-naphthyl)anthracene (ADN) doped with 2,5,8,11-tetra-t-butylperylene (TBP). The electron transport layer 112 may be disposed on the emission layer 110. The electron transport layer 112 may be formed using, for example, a material including tris(8-hydroxyquinolinato)aluminum (Alq₃). The electron injection layer 114 may be disposed on the electron transport layer 112. The electron injection layer 114 may be formed using, for example, a material including lithium fluoride (LiF). The cathode 116 may be disposed on the electron injection layer 114. The cathode 116 may be formed using a metal such as Al or a transparent material such as ITO, IZO, etc. The above-described thin layers may be formed by selecting an appropriate layer forming method such as vacuum deposition, sputtering, various coatings, etc.

In the organic EL device 100 according to this embodiment, driving at a low voltage, high emission efficiency and long life of an organic EL device may be realized by using the material for an organic EL device according to embodiments in a layer of stacked layers disposed between the anode 104 and the emission layer 110. For example, the material for an organic EL device according to embodiments may be used in the hole transport layer 108. Remarkable effects may be obtained by using the material for an organic EL device in a blue emission region and a green emission region. In addition, the material for an organic EL device may be applied in an organic EL apparatus of an active matrix using thin film transistors (TFT).

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

To synthesize the material for an organic EL device, a suitable palladium catalyst, phosphine ligand, and alkaline (basic) reagent may be used. For example, bis(dibenzylideneacetone)palladium(0) may be used as the palladium catalyst, tri-tert-butyl phosphine may be used as the phosphine ligand, and sodium tert-butoxide may be used as the basic reagent.

### EXAMPLES

(Preparation Method)

The material for an organic EL device may be synthesized, for example, as follows.

(Synthesis of Compound 2)

Compound 2 may be synthesized as an example by the following reaction.

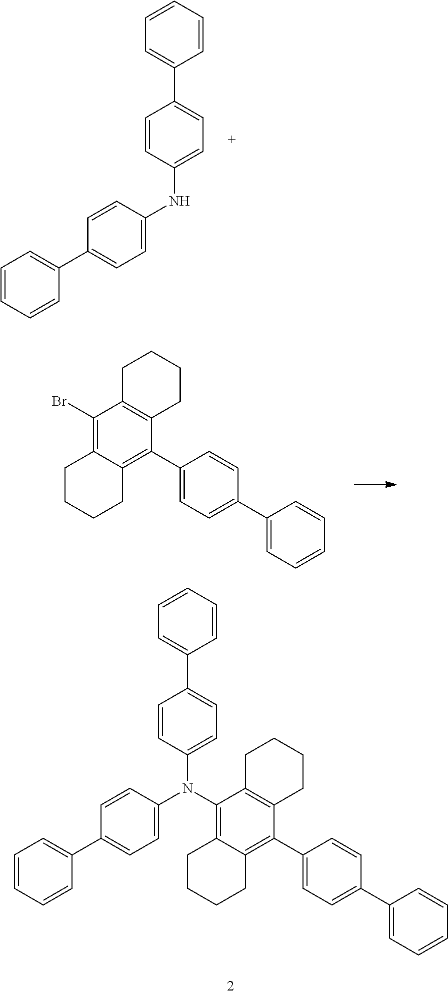 [SMILES: c1ccc(-c2ccc(Nc3ccc(-c4ccccc4)cc3)cc2)cc1 ; Brc1c2c(c(-c3ccc(-c4ccccc4)cc3)c3c1CCCC3)CCCC2 ; c1ccc(-c2ccc(-c3c4c(c(N(c5ccc(-c6ccccc6)cc5)c5ccc(-c6ccccc6)cc5)c5c3CCCC5)CCCC4)cc2)cc1]

Particularly, the amine compound represented above (3 mmol), the bromine substituted compound represented above (3 mmol), a palladium catalyst (0.3 mol), a phosphine ligand (1.2 mol), a basic reagent (12 mmol) and toluene (100 mL) were added to a reaction vessel, followed by charging with nitrogen therein and refluxing while stirring for 20 hours. After cooling, water was poured into the reaction mixture, and an organic layer was extracted. The organic layer thus obtained was dried with magnesium sulfate anhydrous and filtered. The filtrate thus obtained was concentrated by a rotary evaporator, and the crude product thus obtained was separated by silica gel column chromatography. Then, the solid thus obtained was recrystallized to produce a target material as powder type solid with yield of 10% (APCI+: C₅₀H₄₃N, measured value 657).

(Synthesis of Compound 16)

Compound 16 may be synthesized as an example by the following reaction.

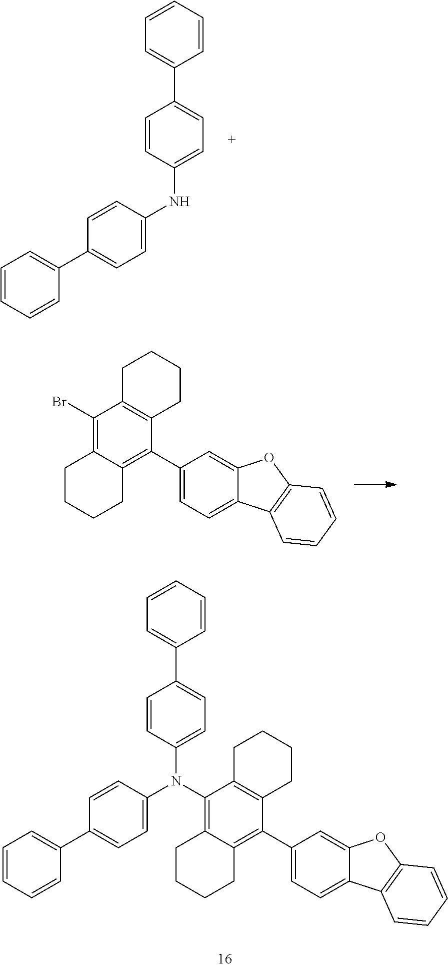 [SMILES: c1ccc(-c2ccc(Nc3ccc(-c4ccccc4)cc3)cc2)cc1 ; Brc1c2c(c(-c3ccc4c(c3)oc3ccccc34)c3c1CCCC3)CCCC2 ; c1ccc(-c2ccc(N(c3ccc(-c4ccccc4)cc3)c3c4c(c(-c5ccc6c(c5)oc5ccccc56)c5c3CCCC5)CCCC4)cc2)cc1]

Particularly, the amine compound represented above (4 mmol), the bromine substituted compound represented above (4 mmol), a palladium catalyst (0.4 mol), a phosphine ligand (1.6 mol), an basic reagent (16 mmol) and toluene (150 mL) were added to a reaction vessel, followed by charging with nitrogen therein and refluxing while stirring for 20 hours. After cooling, water was poured into the reaction mixture, and an organic layer was extracted. The organic layer thus obtained was dried with magnesium sulfate anhydrous and filtered. The filtrate thus obtained was concentrated by a rotary evaporator, and the crude product thus obtained was separated by silica gel column chromatography. Then, the solid thus obtained was recrystallized to produce a target material as powder type solid with yield of 15% (APCI+: C₅₀H₄₁NO, measured value 671).

(Synthesis of Compound 26)

Compound 26 may be synthesized as an example by the following reaction.

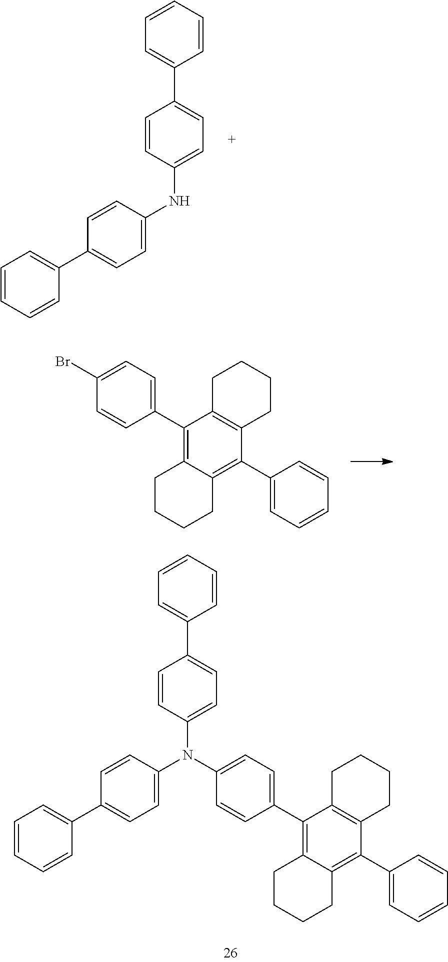

Particularly, the amine compound represented above (3.5 mmol), the bromine substituted compound represented above (3.5 mmol), a palladium catalyst (0.4 mol), a phosphine ligand (1.6 mol), an basic reagent (14 mmol) and toluene (125 mL) were added to a reaction vessel, followed by charging with nitrogen therein and refluxing while stirring for 10 hours. After cooling, water was poured into the reaction mixture, and an organic layer was extracted. The organic layer thus obtained was dried with magnesium sulfate anhydrous and filtered. The filtrate thus obtained was concentrated by a rotary evaporator, and the crude product thus obtained was separated by silica gel column chromatography. Then, the solid thus obtained was recrystallized to produce a target material as powder type solid with yield of 60% (APCI+: C₅₀H₄₃N, measured value 657).

(Synthesis of Compound 91)

Compound 91 may be synthesized as an example by the following reaction.

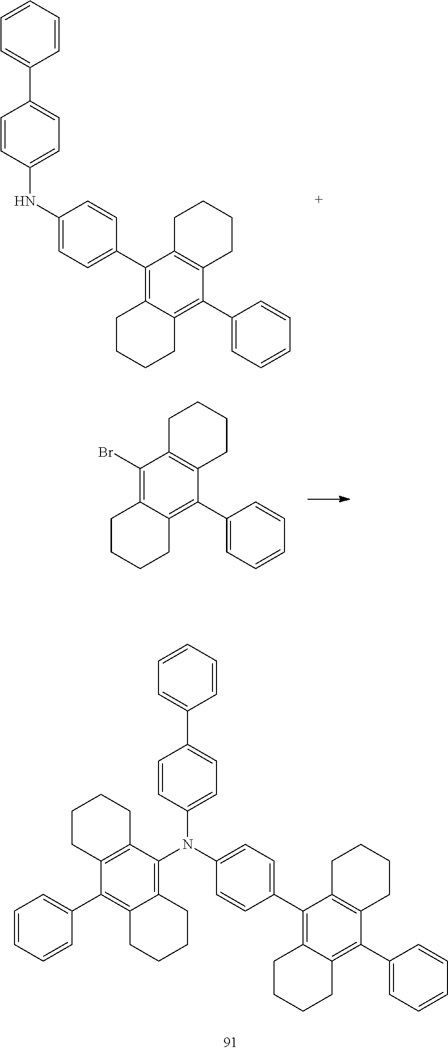 [SMILES: c1ccc(-c2ccc(Nc3ccc(-c4c5c(c(-c6ccccc6)c6c4CCCC6)CCCC5)cc3)cc2)cc1 ; Brc1c2c(c(-c3ccccc3)c3c1CCCC3)CCCC2 ; c1ccc(-c2ccc(N(c3ccc(-c4c5c(c(-c6ccccc6)c6c4CCCC6)CCCC5)cc3)c3c4c(c(-c5ccccc5)c5c3CCCC5)CCCC4)cc2)cc1]

Particularly, the amine compound represented above (1.5 mmol), the bromine substituted compound represented above (1.5 mmol), a palladium catalyst (0.2 mol), a phosphine ligand (0.8 mol), an basic reagent (6 mmol) and toluene (75 mL) were added to a reaction vessel, followed by charging with nitrogen therein and refluxing while stirring for 25 hours. After cooling, water was poured into the reaction mixture, and an organic layer was extracted. The organic layer thus obtained was dried with magnesium sulfate anhydrous and filtered. The filtrate thus obtained was concentrated by a rotary evaporator, and the crude product thus obtained was separated by silica gel column chromatography. Then, the solid thus obtained was recrystallized to produce a target material as powder type solid with yield of 10% (APCI+: C₅₈H₅₅N, measured value 765).

(Synthesis of Compound 101)

Compound 101 may be synthesized as an example by the following reaction.

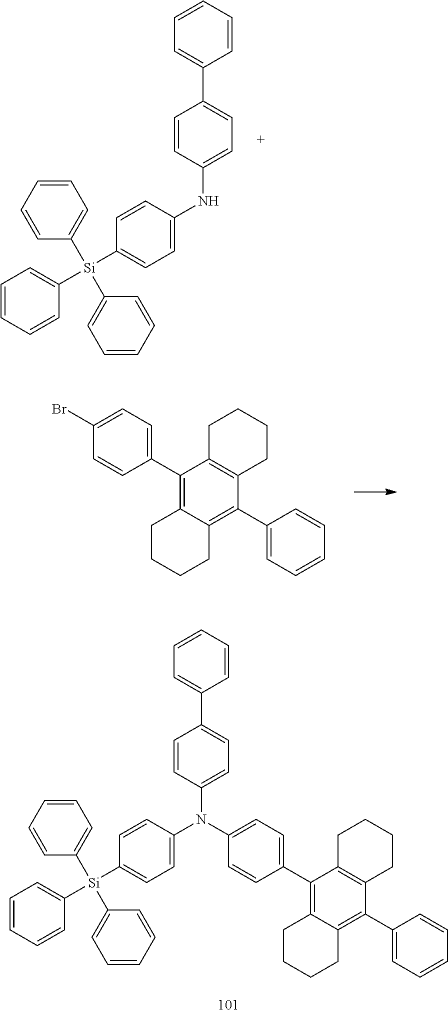 [SMILES: c1ccc(-c2ccc(Nc3ccc([Si](c4ccccc4)(c4ccccc4)c4ccccc4)cc3)cc2)cc1 ; Brc1ccc(-c2c3c(c(-c4ccccc4)c4c2CCCC4)CCCC3)cc1 ; c1ccc(-c2ccc(N(c3ccc(-c4c5c(c(-c6ccccc6)c6c4CCCC6)CCCC5)cc3)c3ccc([Si](c4ccccc4)(c4ccccc4)c4ccccc4)cc3)cc2)cc1]

Particularly, the amine compound represented above (2 mmol), the bromine substituted compound represented above (2 mmol), a palladium catalyst (0.2 mol), a phosphine ligand (0.8 mol), an basic reagent (8 mmol) and toluene (100 mL) were added to a reaction vessel, followed by charging with nitrogen therein and refluxing while stirring for 13 hours. After cooling, water was poured into the reaction mixture, and an organic layer was extracted. The organic layer thus obtained was dried with magnesium sulfate anhydrous and filtered. The filtrate thus obtained was concentrated by a rotary evaporator, and the crude product thus obtained was separated by silica gel column chromatography. Then, the solid thus obtained was recrystallized to produce a target material as powder type solid with yield of 55% (APCI+: C62H53NSi, measured value 839).

Organic EL devices of Examples 1 to 5 were manufactured using the above-described Compounds 2, 16, 26, 91 and 101. In addition, organic EL devices of Comparative Examples 1 and 2 were manufactured using Compounds 121 and 122 for comparison.

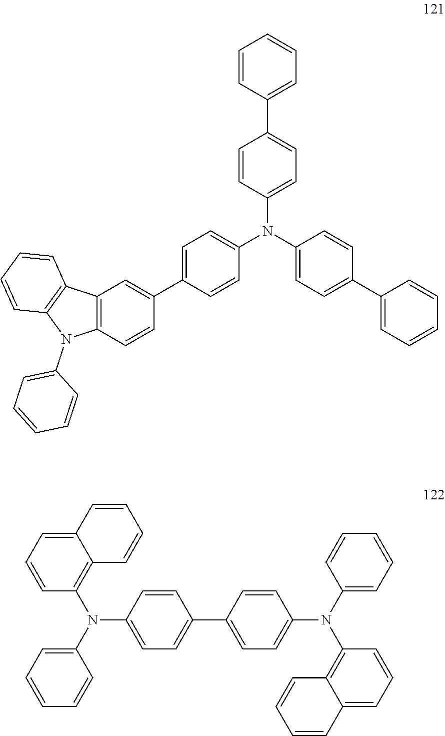 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(N(c2ccc(-c3ccc(N(c4ccccc4)c4cccc5ccccc45)cc3)cc2)c2cccc3ccccc23)cc1]

The organic EL devices according to Examples 1 to 5 and Comparative Examples 1 and 2 were manufactured by the above-described manufacturing method. In this embodiment, the substrate 102 was formed by using a transparent glass substrate, the anode 104 was formed using ITO to a thickness of 150 nm, the hole injection layer 106 was formed using 2-TNATA to a thickness of 60 nm, the hole transport layer 108 was formed to a thickness of 30 nm, the emission layer 110 was formed using ADN doped with 3% TBP to a thickness of 25 nm, the electron transport layer 112 was formed using Algₐ to a thickness of 25 nm, the electron injection layer 114 was formed using LiF to a thickness of 1 nm, and the cathode 116 was formed using Al to a thickness of 100 nm.

With respect to the organic EL devices thus manufactured, the voltage and the current efficiency were measured. The current efficiency denotes values at 10 mA/cm². In addition, life at 1,000 cd/m² was measured. The measured results are illustrated in the following Table 1.

TABLE 1

| | Voltage (V) | Current efficiency (cd/A) | Life (hours) |
|---|---|---|---|
| Example 1 | 6.9 | 8.0 | 2,700 |
| Example 2 | 7.0 | 8.1 | 2,500 |
| Example 3 | 7.3 | 7.5 | 2,200 |
| Example 4 | 7.4 | 7.5 | 2,900 |
| Example 5 | 7.4 | 8.2 | 2,400 |
| Comparative | 7.5 | 6.2 | 1,500 |
| Example 1 | | | |
| Comparative | 8.1 | 5.3 | 1,200 |
| Example 2 | | | |

As clearly shown in Table 1, the organic EL devices according to Examples 1 to 5 were driven at a lower voltage when compared to the organic EL devices according to Comparative Examples 1 and 2. In addition, the organic EL devices of Examples 1 to 5 showed higher current efficiency than the organic EL devices of Comparative Examples 1 and 2. With respect to the life, the organic EL devices of Examples 1 to 5 had a longer life than those of Comparative Examples 1 and 2.

The material for an organic EL device may be an amine compound containing at least one octahydroanthracene group. When such a material is used, the tolerance of the material may increase, and high emission efficiency and long life may be realized. Particularly, the driving at a lower voltage, the high emission efficiency and the long life of an organic EL device may be realized in a blue emission region and a green emission region.

By way of summation and review, an organic EL device may include, for example, an anode, a hole transport layer disposed on the anode, an emission layer disposed on the hole transport layer, an electron transport layer disposed on the emission layer, and a cathode disposed on the electron transport layer. Holes injected from the anode may be injected into the emission layer via the hole transport layer, and electrons may be injected from the cathode, and then injected into the emission layer via the electron transport layer. The holes and the electrons injected into the emission layer may be recombined to generate excitons within the emission layer. The organic EL device emits light by using lights generated by the transition of the excitons to a ground state.

In application of the organic EL device in a display apparatus, the high efficiency of the organic EL device is desirable. It is desirable to obtain a material for a hole transport layer that would allow an organic EL device to be driven at a low voltage and to have improved emission efficiency and long life in a blue emission region and a green emission region.

Embodiments provide a material for an organic EL device capable of being driven at a low voltage and having high emission efficiency and long life, and an organic EL device including the same. Embodiments provide a material for an organic EL device capable of being driven at a low voltage and having high emission efficiency and long life in a blue emission region and a green emission region, and an organic EL device including the same.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope thereof as set forth in the following claims.

What is claimed is:

1. A material for an organic electroluminescence (EL) device represented by the following Formula (1): 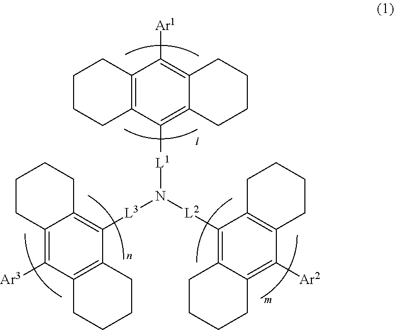
where Ar¹, Ar² and Ar³ are independently a hydrogen atom, a deuterium atom, a halogen atom, a cyano group, a substituted or unsubstituted alkyl group having 1 to 30 carbon atoms, a substituted or unsubstituted alkoxy group having 1 to 30 carbon atoms, a substituted or unsubstituted aryl group having 6 to 30 ring carbon atoms, a substituted or unsubstituted heteroaryl group having 5 to 30 ring atoms, a substituted or unsubstituted triarylsilyl group, or a substituted or unsubstituted trialkylsilyl group,
l, m and n are independently 0 or 1, where the relation of l+m+n≧1 is satisfied, and
L¹, L² and L³ are independently a single bond, a substituted or unsubstituted aryl group having 6 to 30 ring carbon atoms, or a substituted or unsubstituted heteroaryl group having 5 to 30 ring atoms.

2. The material for an organic EL device as claimed in claim 1, wherein the relation of l+m+n=1 is satisfied with respect to l, m and n.

3. The material for an organic EL device as claimed in claim 1, wherein Ar¹, Ar² and Ar³ are independently a hydrogen atom, a substituted or unsubstituted aryl group having 6 to 18 ring carbon atoms, a substituted or unsubstituted carbazolyl group, a substituted or unsubstituted benzofuryl group or a substituted or unsubstituted dibenzothienyl group.

4. The material for an organic EL device as claimed in claim 3, wherein the relation of l+m+n=1 is satisfied with respect to l, m and n.

5. The material for an organic EL device as claimed in claim 1, wherein L¹, L² and L³ are independently the single bond or an aryl group having 6 to 18 ring carbon atoms.

6. The material for an organic EL device as claimed in claim 1, wherein the material includes at least one of the following structures:

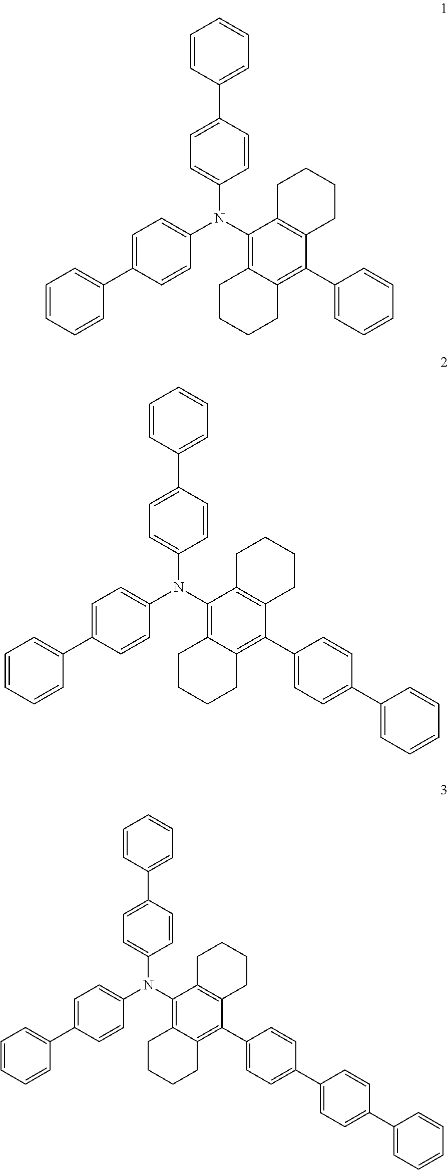 [SMILES: c1ccc(-c2ccc(N(c3ccc(-c4ccccc4)cc3)c3c4c(c(-c5ccccc5)c5c3CCCC5)CCCC4)cc2)cc1 ; c1ccc(-c2ccc(-c3c4c(c(N(c5ccc(-c6ccccc6)cc5)c5ccc(-c6ccccc6)cc5)c5c3CCCC5)CCCC4)cc2)cc1 ; c1ccc(-c2ccc(-c3ccc(-c4c5c(c(N(c6ccc(-c7ccccc7)cc6)c6ccc(-c7ccccc7)cc6)c6c4CCCC6)CCCC5)cc3)cc2)cc1]

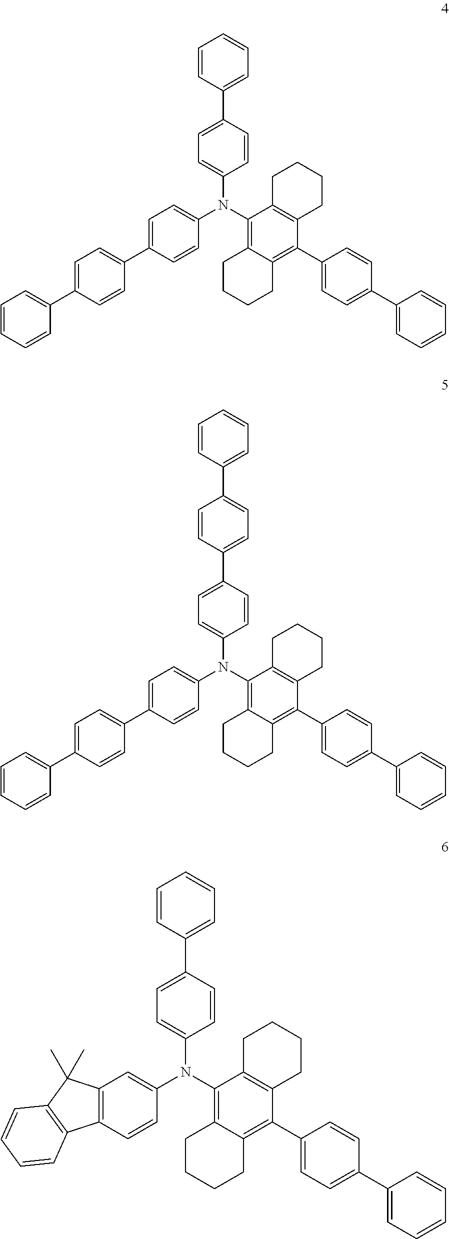 [SMILES: c1ccc(-c2ccc(-c3ccc(N(c4ccc(-c5ccccc5)cc4)c4c5c(c(-c6ccc(-c7ccccc7)cc6)c6c4CCCC6)CCCC5)cc3)cc2)cc1 ; c1ccc(-c2ccc(-c3ccc(N(c4ccc(-c5ccc(-c6ccccc6)cc5)cc4)c4c5c(c(-c6ccc(-c7ccccc7)cc6)c6c4CCCC6)CCCC5)cc3)cc2)cc1 ; CC1(C)c2ccccc2-c2ccc(N(c3ccc(-c4ccccc4)cc3)c3c4c(c(-c5ccc(-c6ccccc6)cc5)c5c3CCCC5)CCCC4)cc21]

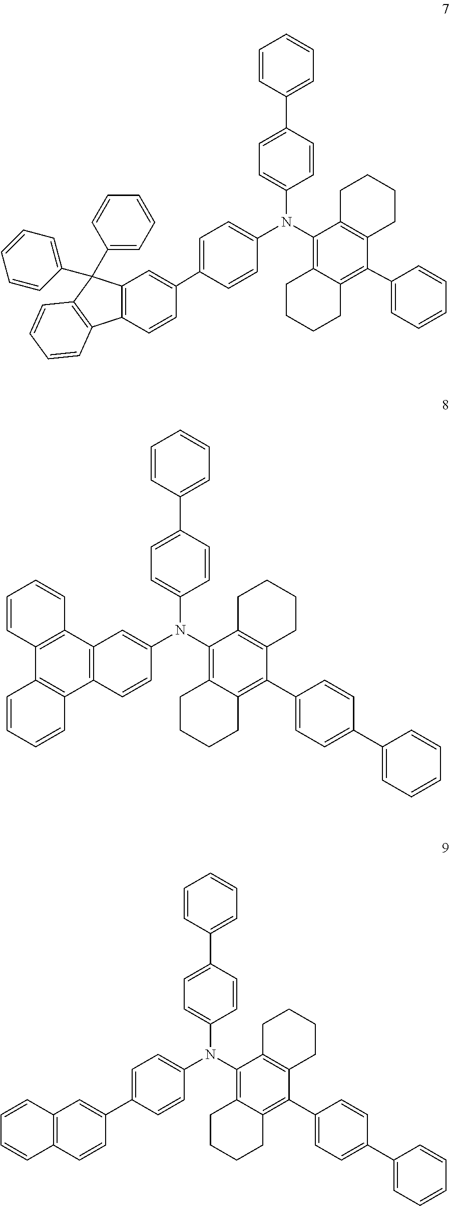 [SMILES: c1ccc(-c2ccc(N(c3ccc(-c4ccc5c(c4)C(c4ccccc4)(c4ccccc4)c4ccccc4-5)cc3)c3c4c(c(-c5ccccc5)c5c3CCCC5)CCCC4)cc2)cc1 ; c1ccc(-c2ccc(-c3c4c(c(N(c5ccc(-c6ccccc6)cc5)c5ccc6c7ccccc7c7ccccc7c6c5)c5c3CCCC5)CCCC4)cc2)cc1 ; c1ccc(-c2ccc(-c3c4c(c(N(c5ccc(-c6ccccc6)cc5)c5ccc(-c6ccc7ccccc7c6)cc5)c5c3CCCC5)CCCC4)cc2)cc1]

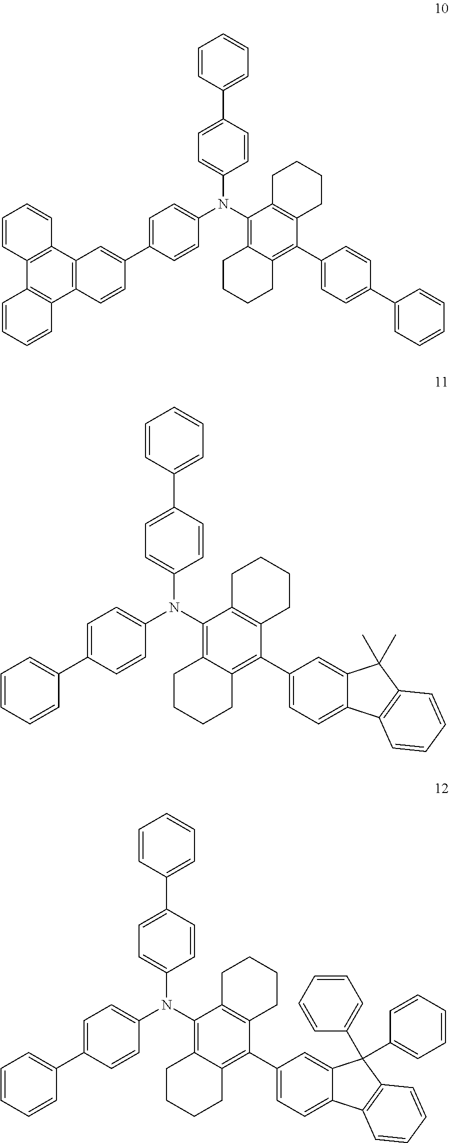 [SMILES: c1ccc(-c2ccc(-c3c4c(c(N(c5ccc(-c6ccccc6)cc5)c5ccc(-c6ccc7c8ccccc8c8ccccc8c7c6)cc5)c5c3CCCC5)CCCC4)cc2)cc1 ; CC1(C)c2ccccc2-c2ccc(-c3c4c(c(N(c5ccc(-c6ccccc6)cc5)c5ccc(-c6ccccc6)cc5)c5c3CCCC5)CCCC4)cc21 ; c1ccc(-c2ccc(N(c3ccc(-c4ccccc4)cc3)c3c4c(c(-c5ccc6c(c5)C(c5ccccc5)(c5ccccc5)c5ccccc5-6)c5c3CCCC5)CCCC4)cc2)cc1]

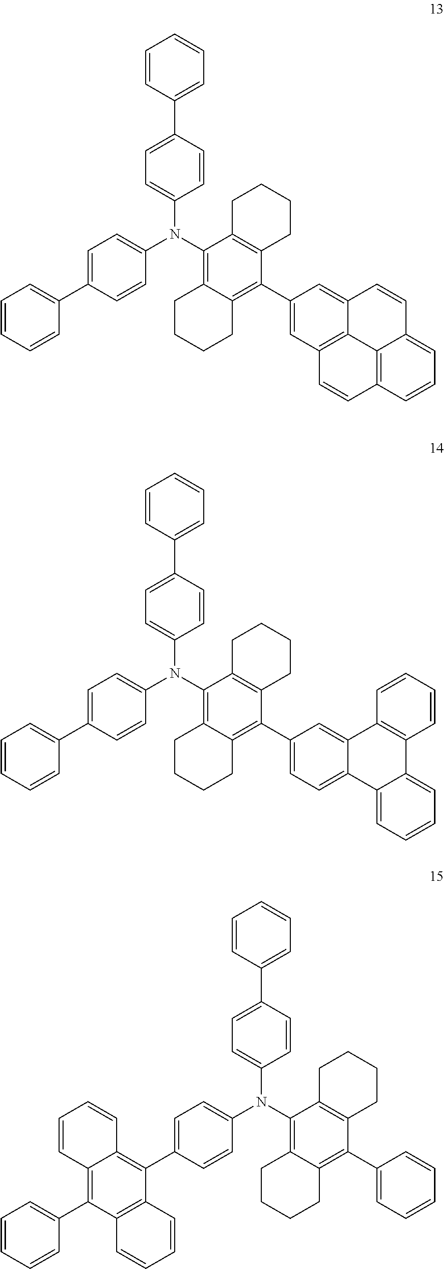 [SMILES: c1ccc(-c2ccc(N(c3ccc(-c4ccccc4)cc3)c3c4c(c(-c5cc6ccc7cccc8ccc(c5)c6c78)c5c3CCCC5)CCCC4)cc2)cc1 ; c1ccc(-c2ccc(N(c3ccc(-c4ccccc4)cc3)c3c4c(c(-c5ccc6c7ccccc7c7ccccc7c6c5)c5c3CCCC5)CCCC4)cc2)cc1 ; c1ccc(-c2ccc(N(c3ccc(-c4c5ccccc5c(-c5ccccc5)c5ccccc45)cc3)c3c4c(c(-c5ccccc5)c5c3CCCC5)CCCC4)cc2)cc1]

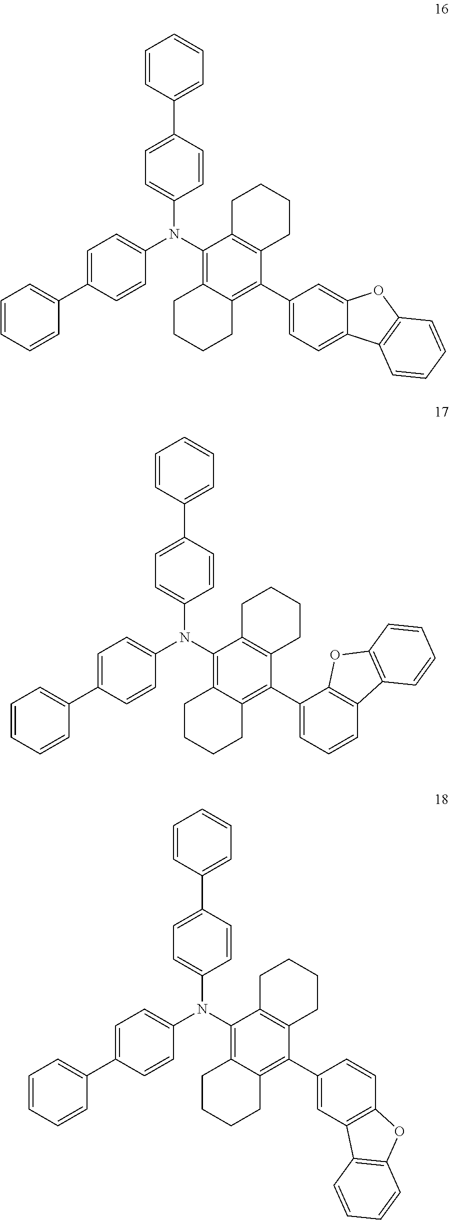 [SMILES: c1ccc(-c2ccc(N(c3ccc(-c4ccccc4)cc3)c3c4c(c(-c5ccc6c(c5)oc5ccccc56)c5c3CCCC5)CCCC4)cc2)cc1 ; c1ccc(-c2ccc(N(c3ccc(-c4ccccc4)cc3)c3c4c(c(-c5cccc6c5oc5ccccc56)c5c3CCCC5)CCCC4)cc2)cc1 ; c1ccc(-c2ccc(N(c3ccc(-c4ccccc4)cc3)c3c4c(c(-c5ccc6oc7ccccc7c6c5)c5c3CCCC5)CCCC4)cc2)cc1]

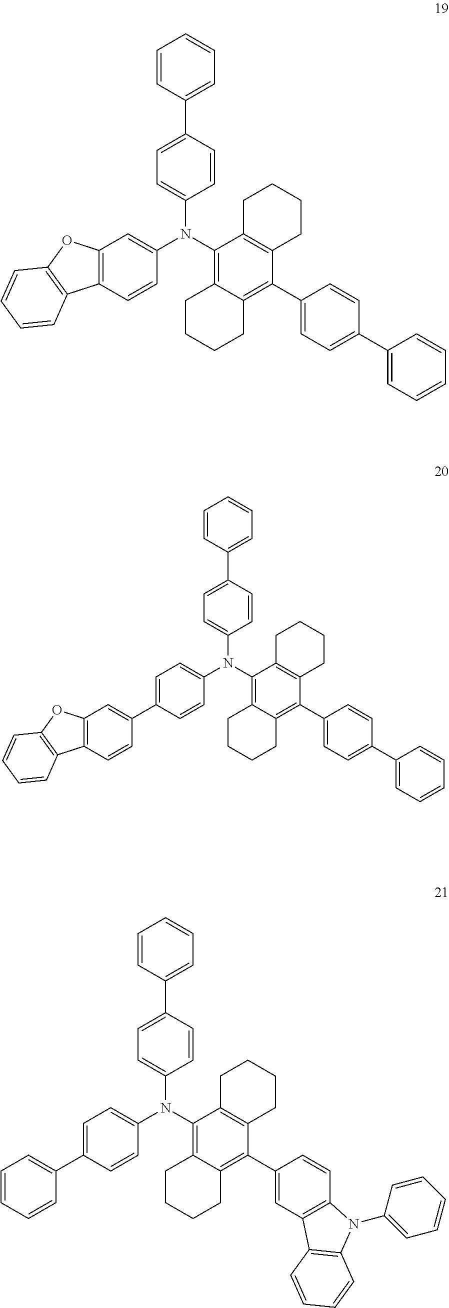 [SMILES: c1ccc(-c2ccc(-c3c4c(c(N(c5ccc(-c6ccccc6)cc5)c5ccc6c(c5)oc5ccccc56)c5c3CCCC5)CCCC4)cc2)cc1 ; c1ccc(-c2ccc(-c3c4c(c(N(c5ccc(-c6ccccc6)cc5)c5ccc(-c6ccc7c(c6)oc6ccccc67)cc5)c5c3CCCC5)CCCC4)cc2)cc1 ; c1ccc(-c2ccc(N(c3ccc(-c4ccccc4)cc3)c3c4c(c(-c5ccc6c(c5)c5ccccc5n6-c5ccccc5)c5c3CCCC5)CCCC4)cc2)cc1]

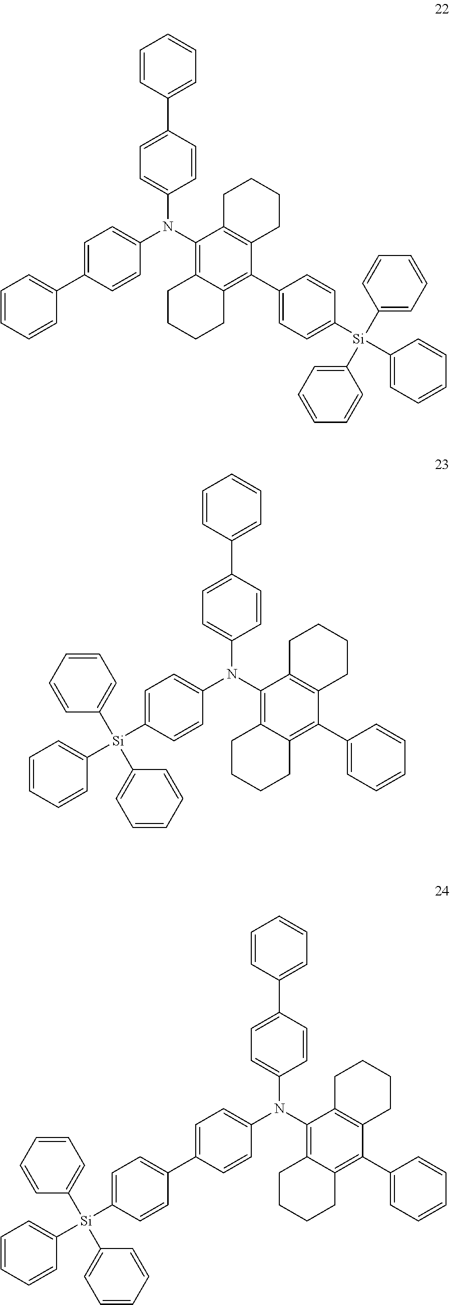 [SMILES: c1ccc(-c2ccc(N(c3ccc(-c4ccccc4)cc3)c3c4c(c(-c5ccc([Si](c6ccccc6)(c6ccccc6)c6ccccc6)cc5)c5c3CCCC5)CCCC4)cc2)cc1 ; c1ccc(-c2ccc(N(c3ccc([Si](c4ccccc4)(c4ccccc4)c4ccccc4)cc3)c3c4c(c(-c5ccccc5)c5c3CCCC5)CCCC4)cc2)cc1 ; c1ccc(-c2ccc(N(c3ccc(-c4ccc([Si](c5ccccc5)(c5ccccc5)c5ccccc5)cc4)cc3)c3c4c(c(-c5ccccc5)c5c3CCCC5)CCCC4)cc2)cc1]

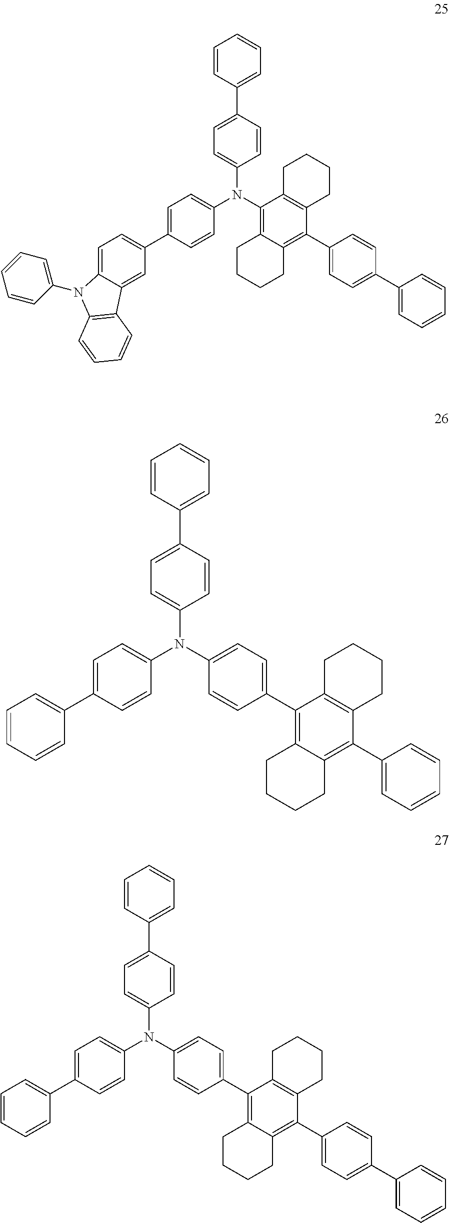 [SMILES: c1ccc(-c2ccc(-c3c4c(c(N(c5ccc(-c6ccccc6)cc5)c5ccc(-c6ccc7c(c6)c6ccccc6n7-c6ccccc6)cc5)c5c3CCCC5)CCCC4)cc2)cc1 ; c1ccc(-c2ccc(N(c3ccc(-c4ccccc4)cc3)c3ccc(-c4c5c(c(-c6ccccc6)c6c4CCCC6)CCCC5)cc3)cc2)cc1 ; c1ccc(-c2ccc(-c3c4c(c(-c5ccc(N(c6ccc(-c7ccccc7)cc6)c6ccc(-c7ccccc7)cc6)cc5)c5c3CCCC5)CCCC4)cc2)cc1]

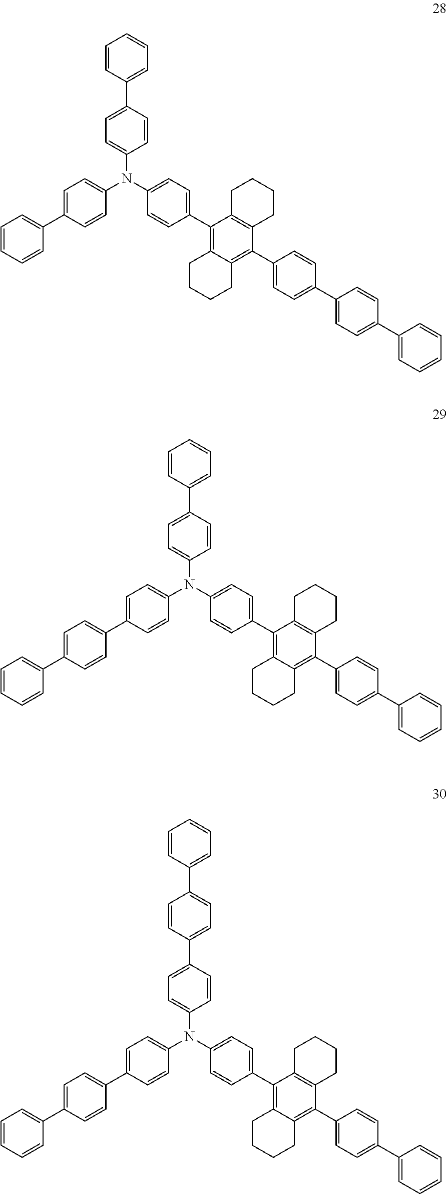 [SMILES: c1ccc(-c2ccc(-c3ccc(-c4c5c(c(-c6ccc(N(c7ccc(-c8ccccc8)cc7)c7ccc(-c8ccccc8)cc7)cc6)c6c4CCCC6)CCCC5)cc3)cc2)cc1 ; c1ccc(-c2ccc(-c3ccc(N(c4ccc(-c5ccccc5)cc4)c4ccc(-c5c6c(c(-c7ccc(-c8ccccc8)cc7)c7c5CCCC7)CCCC6)cc4)cc3)cc2)cc1 ; c1ccc(-c2ccc(-c3ccc(N(c4ccc(-c5ccc(-c6ccccc6)cc5)cc4)c4ccc(-c5c6c(c(-c7ccc(-c8ccccc8)cc7)c7c5CCCC7)CCCC6)cc4)cc3)cc2)cc1]

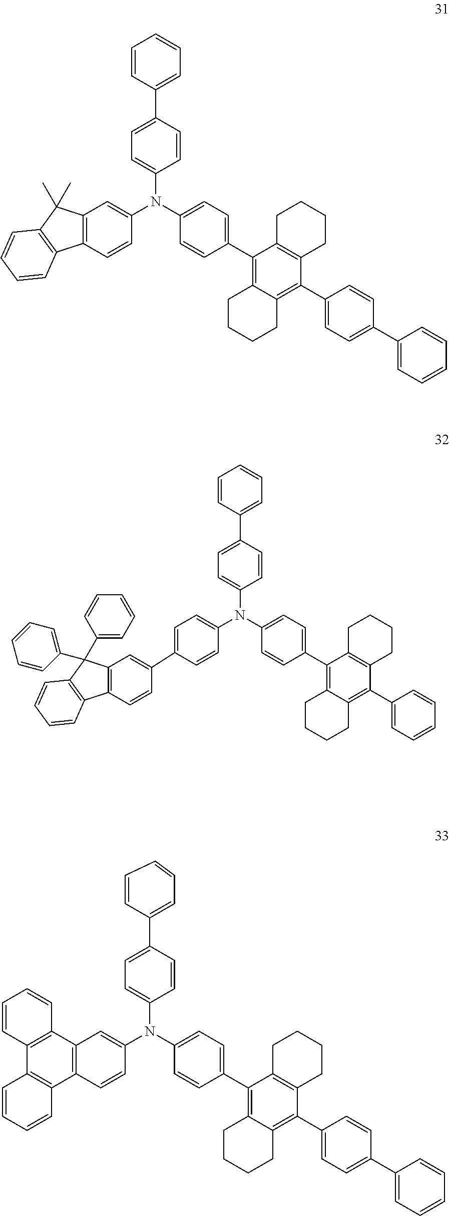 [SMILES: CC1(C)c2ccccc2-c2ccc(N(c3ccc(-c4ccccc4)cc3)c3ccc(-c4c5c(c(-c6ccc(-c7ccccc7)cc6)c6c4CCCC6)CCCC5)cc3)cc21 ; c1ccc(-c2ccc(N(c3ccc(-c4ccc5c(c4)C(c4ccccc4)(c4ccccc4)c4ccccc4-5)cc3)c3ccc(-c4c5c(c(-c6ccccc6)c6c4CCCC6)CCCC5)cc3)cc2)cc1 ; c1ccc(-c2ccc(-c3c4c(c(-c5ccc(N(c6ccc(-c7ccccc7)cc6)c6ccc7c8ccccc8c8ccccc8c7c6)cc5)c5c3CCCC5)CCCC4)cc2)cc1]

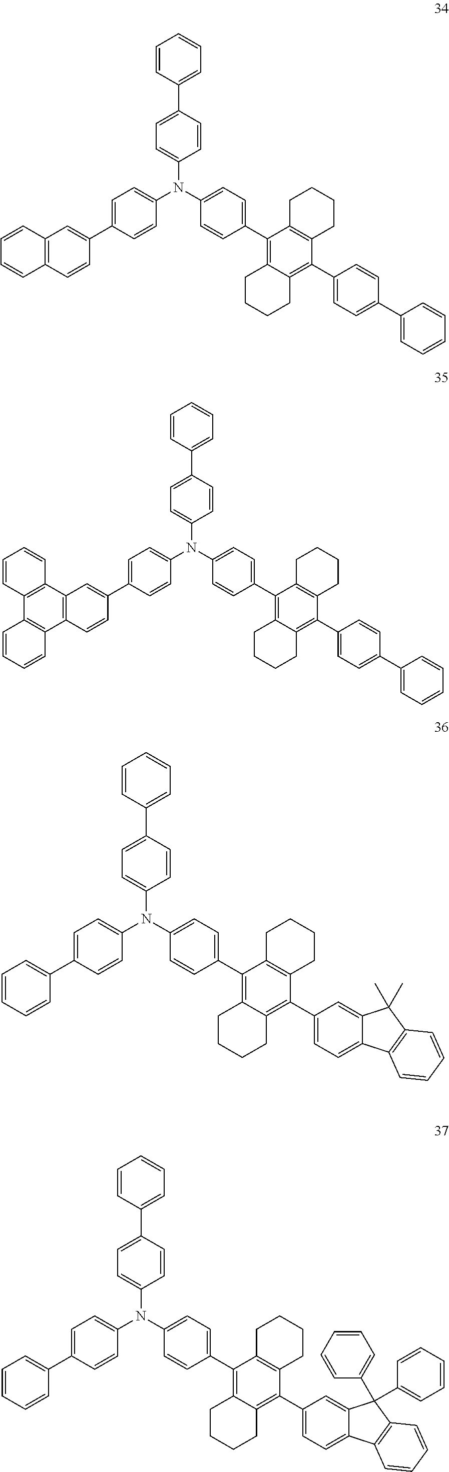 [SMILES: c1ccc(-c2ccc(-c3c4c(c(-c5ccc(N(c6ccc(-c7ccccc7)cc6)c6ccc(-c7ccc8ccccc8c7)cc6)cc5)c5c3CCCC5)CCCC4)cc2)cc1 ; c1ccc(-c2ccc(-c3c4c(c(-c5ccc(N(c6ccc(-c7ccccc7)cc6)c6ccc(-c7ccc8c9ccccc9c9ccccc9c8c7)cc6)cc5)c5c3CCCC5)CCCC4)cc2)cc1 ; CC1(C)c2ccccc2-c2ccc(-c3c4c(c(-c5ccc(N(c6ccc(-c7ccccc7)cc6)c6ccc(-c7ccccc7)cc6)cc5)c5c3CCCC5)CCCC4)cc21 ; c1ccc(-c2ccc(N(c3ccc(-c4ccccc4)cc3)c3ccc(-c4c5c(c(-c6ccc7c(c6)C(c6ccccc6)(c6ccccc6)c6ccccc6-7)c6c4CCCC6)CCCC5)cc3)cc2)cc1]

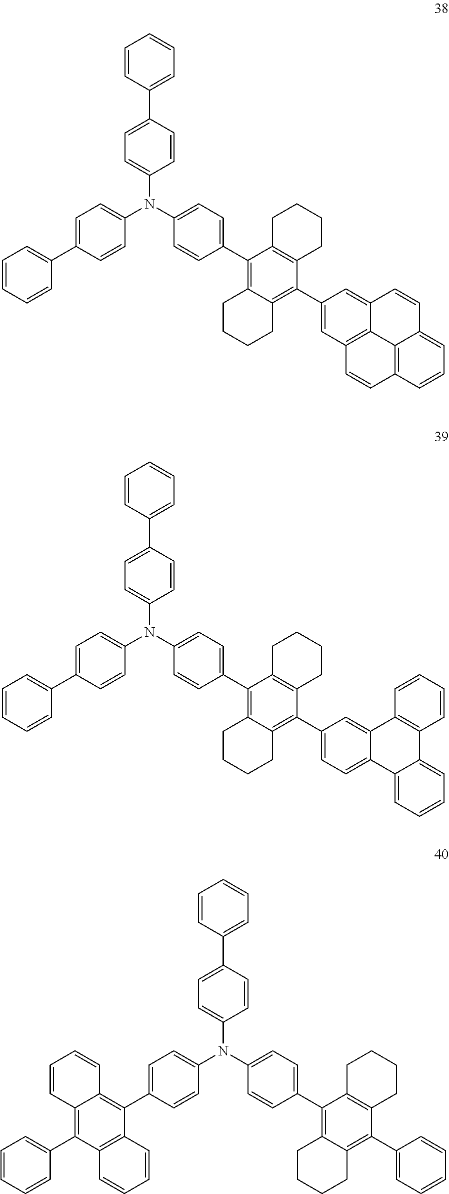 [SMILES: c1ccc(-c2ccc(N(c3ccc(-c4ccccc4)cc3)c3ccc(-c4c5c(c(-c6cc7ccc8cccc9ccc(c6)c7c89)c6c4CCCC6)CCCC5)cc3)cc2)cc1 ; c1ccc(-c2ccc(N(c3ccc(-c4ccccc4)cc3)c3ccc(-c4c5c(c(-c6ccc7c8ccccc8c8ccccc8c7c6)c6c4CCCC6)CCCC5)cc3)cc2)cc1 ; c1ccc(-c2ccc(N(c3ccc(-c4c5c(c(-c6ccccc6)c6c4CCCC6)CCCC5)cc3)c3ccc(-c4c5ccccc5c(-c5ccccc5)c5ccccc45)cc3)cc2)cc1]

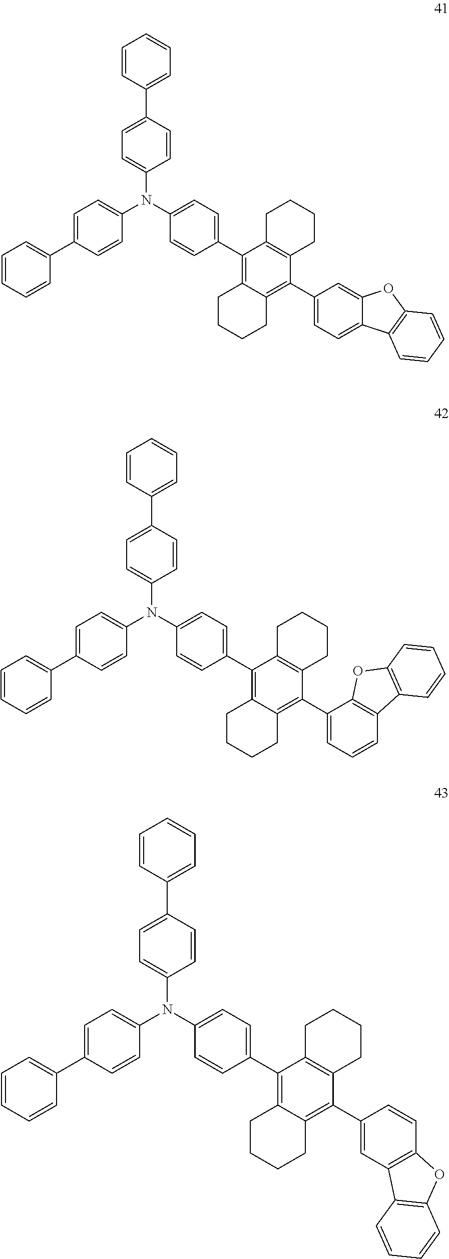 [SMILES: c1ccc(-c2ccc(N(c3ccc(-c4ccccc4)cc3)c3ccc(-c4c5c(c(-c6ccc7c(c6)oc6ccccc67)c6c4CCCC6)CCCC5)cc3)cc2)cc1 ; c1ccc(-c2ccc(N(c3ccc(-c4ccccc4)cc3)c3ccc(-c4c5c(c(-c6cccc7c6oc6ccccc67)c6c4CCCC6)CCCC5)cc3)cc2)cc1 ; c1ccc(-c2ccc(N(c3ccc(-c4ccccc4)cc3)c3ccc(-c4c5c(c(-c6ccc7oc8ccccc8c7c6)c6c4CCCC6)CCCC5)cc3)cc2)cc1]

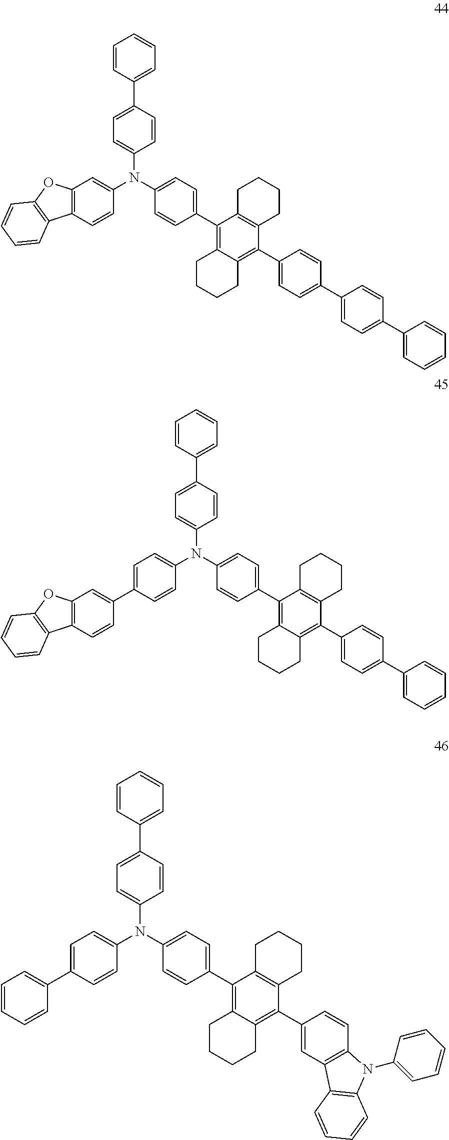 [SMILES: c1ccc(-c2ccc(-c3ccc(-c4c5c(c(-c6ccc(N(c7ccc(-c8ccccc8)cc7)c7ccc8c(c7)oc7ccccc78)cc6)c6c4CCCC6)CCCC5)cc3)cc2)cc1 ; c1ccc(-c2ccc(-c3c4c(c(-c5ccc(N(c6ccc(-c7ccccc7)cc6)c6ccc(-c7ccc8c(c7)oc7ccccc78)cc6)cc5)c5c3CCCC5)CCCC4)cc2)cc1 ; c1ccc(-c2ccc(N(c3ccc(-c4ccccc4)cc3)c3ccc(-c4c5c(c(-c6ccc7c(c6)c6ccccc6n7-c6ccccc6)c6c4CCCC6)CCCC5)cc3)cc2)cc1]

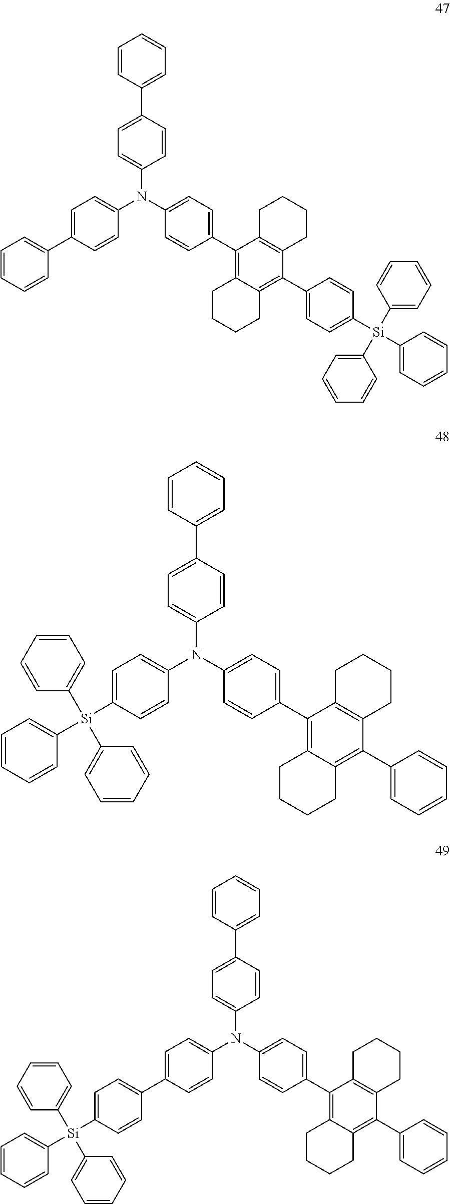 [SMILES: c1ccc(-c2ccc(N(c3ccc(-c4ccccc4)cc3)c3ccc(-c4c5c(c(-c6ccc([Si](c7ccccc7)(c7ccccc7)c7ccccc7)cc6)c6c4CCCC6)CCCC5)cc3)cc2)cc1 ; c1ccc(-c2ccc(N(c3ccc(-c4c5c(c(-c6ccccc6)c6c4CCCC6)CCCC5)cc3)c3ccc([Si](c4ccccc4)(c4ccccc4)c4ccccc4)cc3)cc2)cc1 ; c1ccc(-c2ccc(N(c3ccc(-c4ccc([Si](c5ccccc5)(c5ccccc5)c5ccccc5)cc4)cc3)c3ccc(-c4c5c(c(-c6ccccc6)c6c4CCCC6)CCCC5)cc3)cc2)cc1]

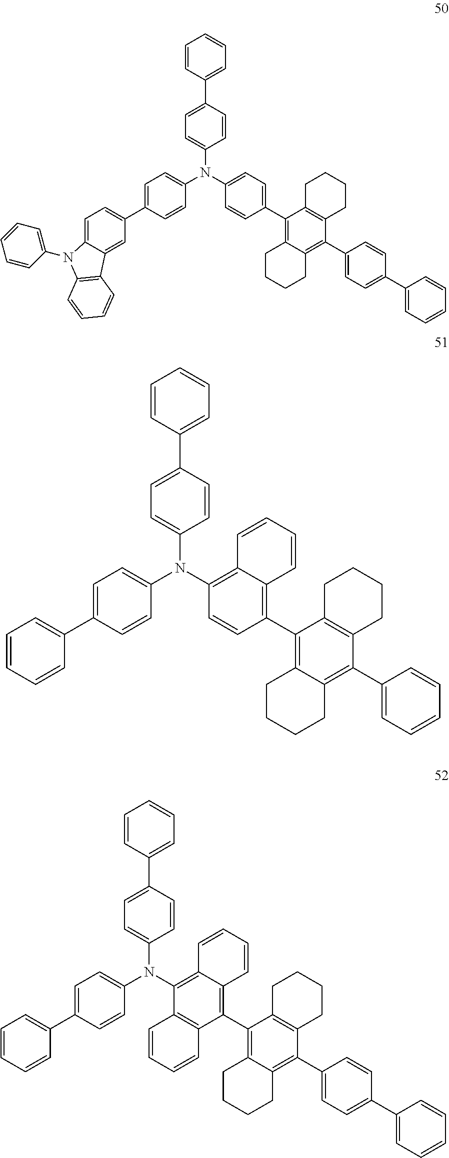 [SMILES: c1ccc(-c2ccc(-c3c4c(c(-c5ccc(N(c6ccc(-c7ccccc7)cc6)c6ccc(-c7ccc8c(c7)c7ccccc7n8-c7ccccc7)cc6)cc5)c5c3CCCC5)CCCC4)cc2)cc1 ; c1ccc(-c2ccc(N(c3ccc(-c4ccccc4)cc3)c3ccc(-c4c5c(c(-c6ccccc6)c6c4CCCC6)CCCC5)c4ccccc34)cc2)cc1 ; c1ccc(-c2ccc(-c3c4c(c(-c5c6ccccc6c(N(c6ccc(-c7ccccc7)cc6)c6ccc(-c7ccccc7)cc6)c6ccccc56)c5c3CCCC5)CCCC4)cc2)cc1]

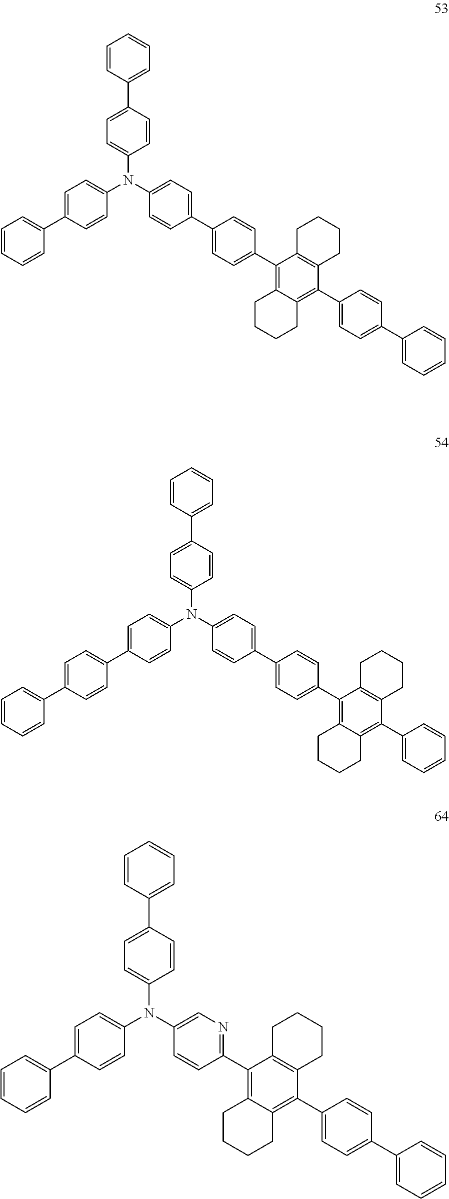 [SMILES: c1ccc(-c2ccc(-c3c4c(c(-c5ccc(-c6ccc(N(c7ccc(-c8ccccc8)cc7)c7ccc(-c8ccccc8)cc7)cc6)cc5)c5c3CCCC5)CCCC4)cc2)cc1 ; c1ccc(-c2ccc(-c3ccc(N(c4ccc(-c5ccccc5)cc4)c4ccc(-c5ccc(-c6c7c(c(-c8ccccc8)c8c6CCCC8)CCCC7)cc5)cc4)cc3)cc2)cc1 ; c1ccc(-c2ccc(-c3c4c(c(-c5ccc(N(c6ccc(-c7ccccc7)cc6)c6ccc(-c7ccccc7)cc6)cn5)c5c3CCCC5)CCCC4)cc2)cc1]

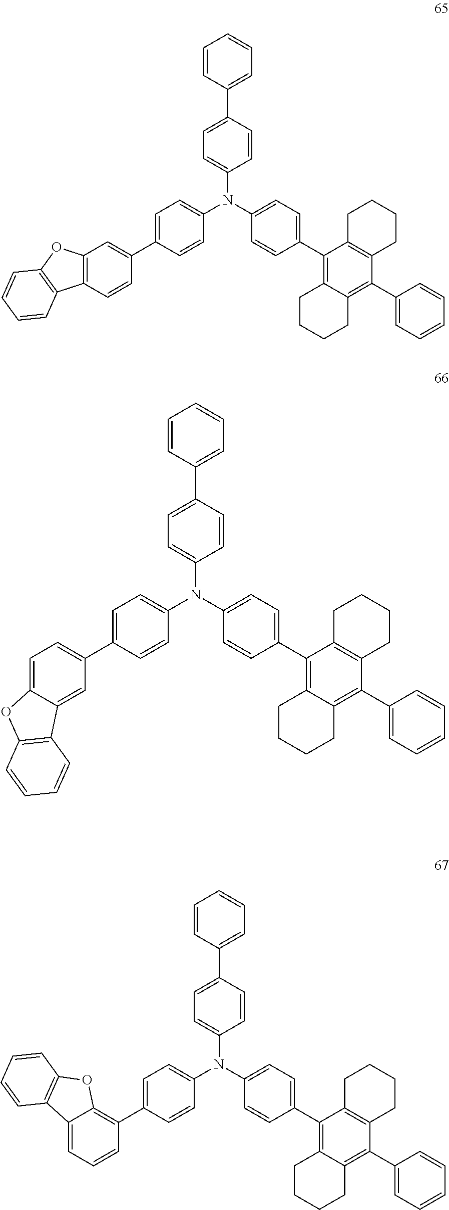 [SMILES: c1ccc(-c2ccc(N(c3ccc(-c4ccc5c(c4)oc4ccccc45)cc3)c3ccc(-c4c5c(c(-c6ccccc6)c6c4CCCC6)CCCC5)cc3)cc2)cc1 ; c1ccc(-c2ccc(N(c3ccc(-c4ccc5oc6ccccc6c5c4)cc3)c3ccc(-c4c5c(c(-c6ccccc6)c6c4CCCC6)CCCC5)cc3)cc2)cc1 ; c1ccc(-c2ccc(N(c3ccc(-c4c5c(c(-c6ccccc6)c6c4CCCC6)CCCC5)cc3)c3ccc(-c4cccc5c4oc4ccccc45)cc3)cc2)cc1]

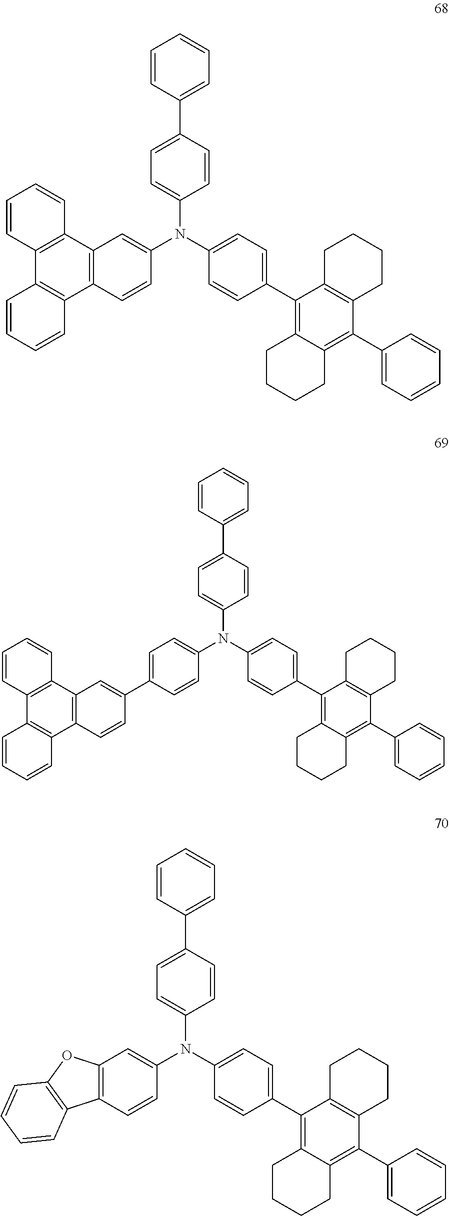 [SMILES: c1ccc(-c2ccc(N(c3ccc(-c4c5c(c(-c6ccccc6)c6c4CCCC6)CCCC5)cc3)c3ccc4c5ccccc5c5ccccc5c4c3)cc2)cc1 ; c1ccc(-c2ccc(N(c3ccc(-c4ccc5c6ccccc6c6ccccc6c5c4)cc3)c3ccc(-c4c5c(c(-c6ccccc6)c6c4CCCC6)CCCC5)cc3)cc2)cc1 ; c1ccc(-c2ccc(N(c3ccc(-c4c5c(c(-c6ccccc6)c6c4CCCC6)CCCC5)cc3)c3ccc4c(c3)oc3ccccc34)cc2)cc1]

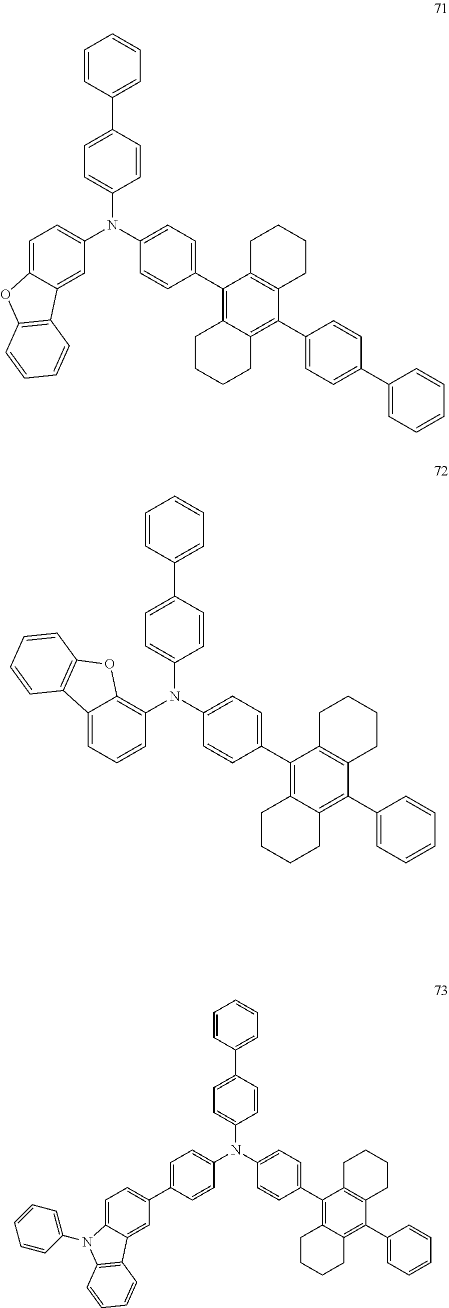 [SMILES: c1ccc(-c2ccc(-c3c4c(c(-c5ccc(N(c6ccc(-c7ccccc7)cc6)c6ccc7oc8ccccc8c7c6)cc5)c5c3CCCC5)CCCC4)cc2)cc1 ; c1ccc(-c2ccc(N(c3ccc(-c4c5c(c(-c6ccccc6)c6c4CCCC6)CCCC5)cc3)c3cccc4c3oc3ccccc34)cc2)cc1 ; c1ccc(-c2ccc(N(c3ccc(-c4ccc5c(c4)c4ccccc4n5-c4ccccc4)cc3)c3ccc(-c4c5c(c(-c6ccccc6)c6c4CCCC6)CCCC5)cc3)cc2)cc1]

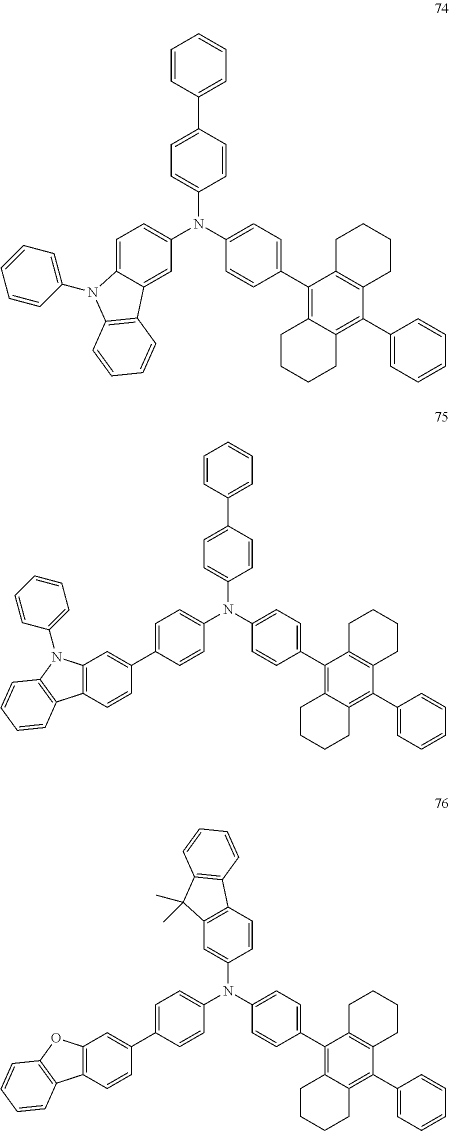 [SMILES: c1ccc(-c2ccc(N(c3ccc(-c4c5c(c(-c6ccccc6)c6c4CCCC6)CCCC5)cc3)c3ccc4c(c3)c3ccccc3n4-c3ccccc3)cc2)cc1 ; c1ccc(-c2ccc(N(c3ccc(-c4ccc5c6ccccc6n(-c6ccccc6)c5c4)cc3)c3ccc(-c4c5c(c(-c6ccccc6)c6c4CCCC6)CCCC5)cc3)cc2)cc1 ; CC1(C)c2ccccc2-c2ccc(N(c3ccc(-c4ccc5c(c4)oc4ccccc45)cc3)c3ccc(-c4c5c(c(-c6ccccc6)c6c4CCCC6)CCCC5)cc3)cc21]

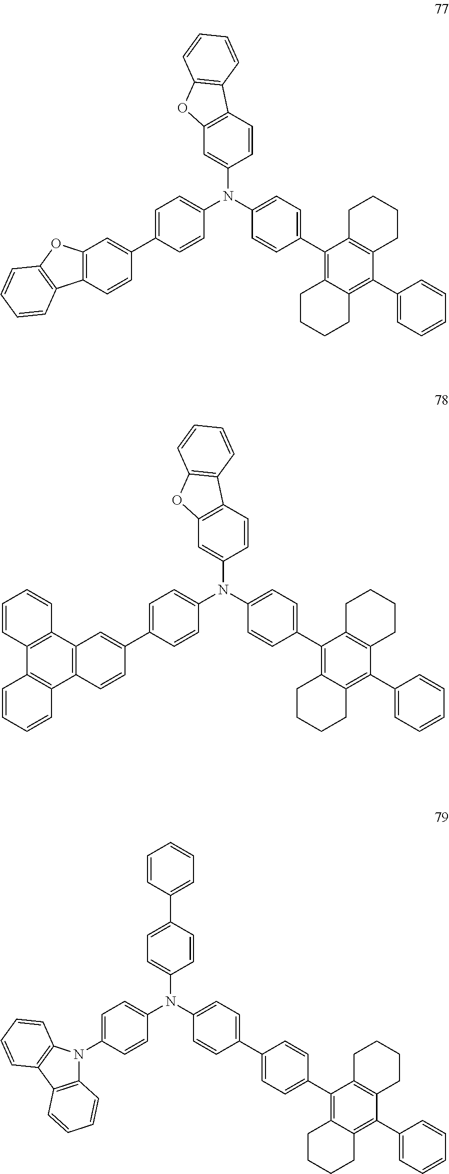 [SMILES: c1ccc(-c2c3c(c(-c4ccc(N(c5ccc(-c6ccc7c(c6)oc6ccccc67)cc5)c5ccc6c(c5)oc5ccccc56)cc4)c4c2CCCC4)CCCC3)cc1 ; c1ccc(-c2c3c(c(-c4ccc(N(c5ccc(-c6ccc7c8ccccc8c8ccccc8c7c6)cc5)c5ccc6c(c5)oc5ccccc56)cc4)c4c2CCCC4)CCCC3)cc1 ; c1ccc(-c2ccc(N(c3ccc(-c4ccc(-c5c6c(c(-c7ccccc7)c7c5CCCC7)CCCC6)cc4)cc3)c3ccc(-n4c5ccccc5c5ccccc54)cc3)cc2)cc1]

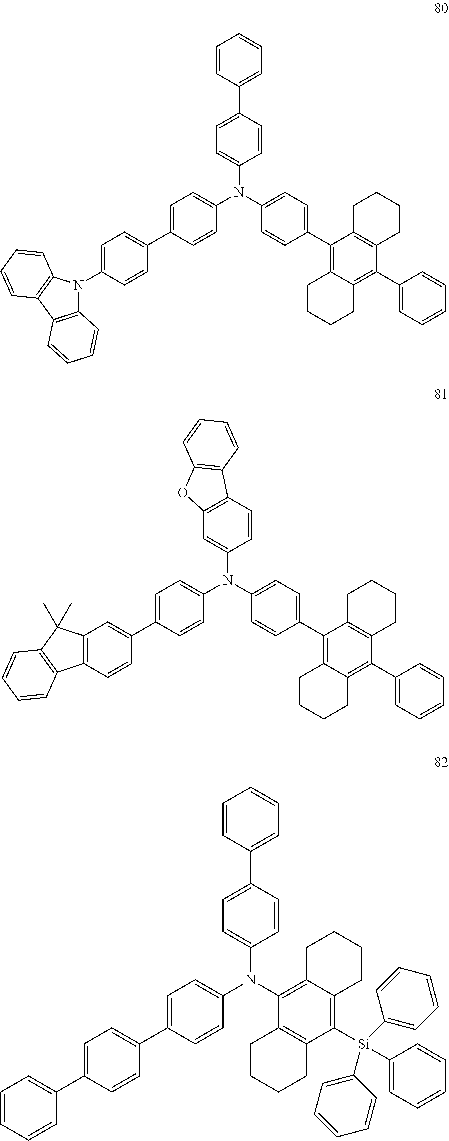 [SMILES: c1ccc(-c2ccc(N(c3ccc(-c4ccc(-n5c6ccccc6c6ccccc65)cc4)cc3)c3ccc(-c4c5c(c(-c6ccccc6)c6c4CCCC6)CCCC5)cc3)cc2)cc1 ; CC1(C)c2ccccc2-c2ccc(-c3ccc(N(c4ccc(-c5c6c(c(-c7ccccc7)c7c5CCCC7)CCCC6)cc4)c4ccc5c(c4)oc4ccccc45)cc3)cc21 ; c1ccc(-c2ccc(-c3ccc(N(c4ccc(-c5ccccc5)cc4)c4c5c(c([Si](c6ccccc6)(c6ccccc6)c6ccccc6)c6c4CCCC6)CCCC5)cc3)cc2)cc1]

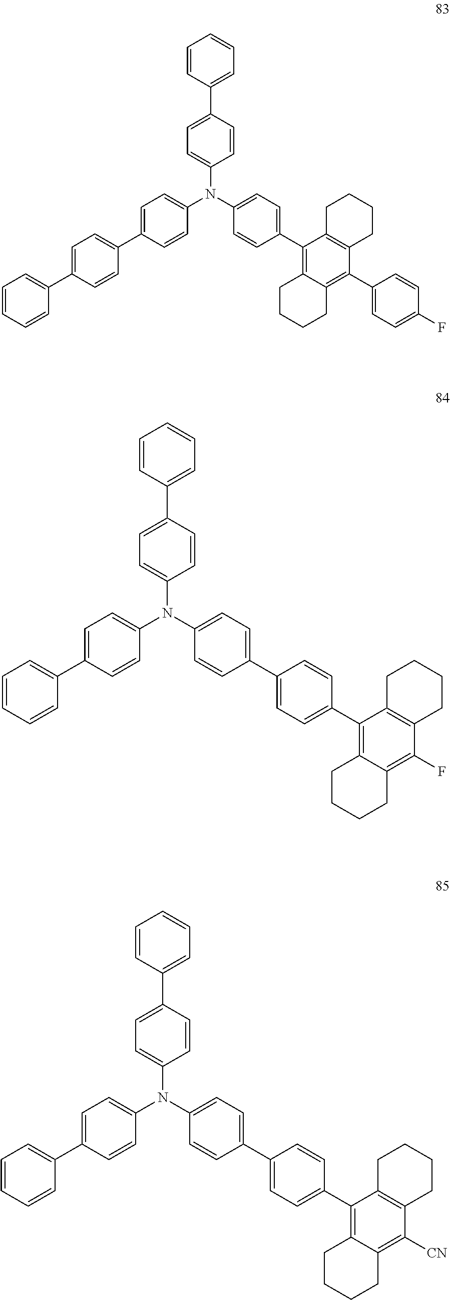 [SMILES: Fc1ccc(-c2c3c(c(-c4ccc(N(c5ccc(-c6ccccc6)cc5)c5ccc(-c6ccc(-c7ccccc7)cc6)cc5)cc4)c4c2CCCC4)CCCC3)cc1 ; Fc1c2c(c(-c3ccc(-c4ccc(N(c5ccc(-c6ccccc6)cc5)c5ccc(-c6ccccc6)cc5)cc4)cc3)c3c1CCCC3)CCCC2 ; N#Cc1c2c(c(-c3ccc(-c4ccc(N(c5ccc(-c6ccccc6)cc5)c5ccc(-c6ccccc6)cc5)cc4)cc3)c3c1CCCC3)CCCC2]

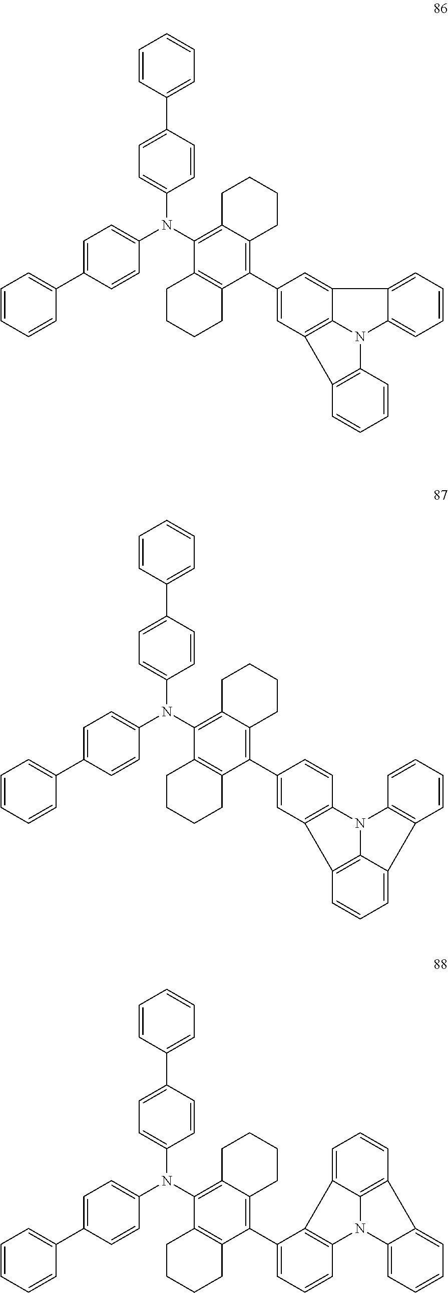 [SMILES: c1ccc(-c2ccc(N(c3ccc(-c4ccccc4)cc3)c3c4c(c(-c5cc6c7ccccc7n7c8ccccc8c(c5)c67)c5c3CCCC5)CCCC4)cc2)cc1 ; c1ccc(-c2ccc(N(c3ccc(-c4ccccc4)cc3)c3c4c(c(-c5ccc6c(c5)c5cccc7c8ccccc8n6c75)c5c3CCCC5)CCCC4)cc2)cc1 ; c1ccc(-c2ccc(N(c3ccc(-c4ccccc4)cc3)c3c4c(c(-c5cccc6c5c5cccc7c8ccccc8n6c75)c5c3CCCC5)CCCC4)cc2)cc1]

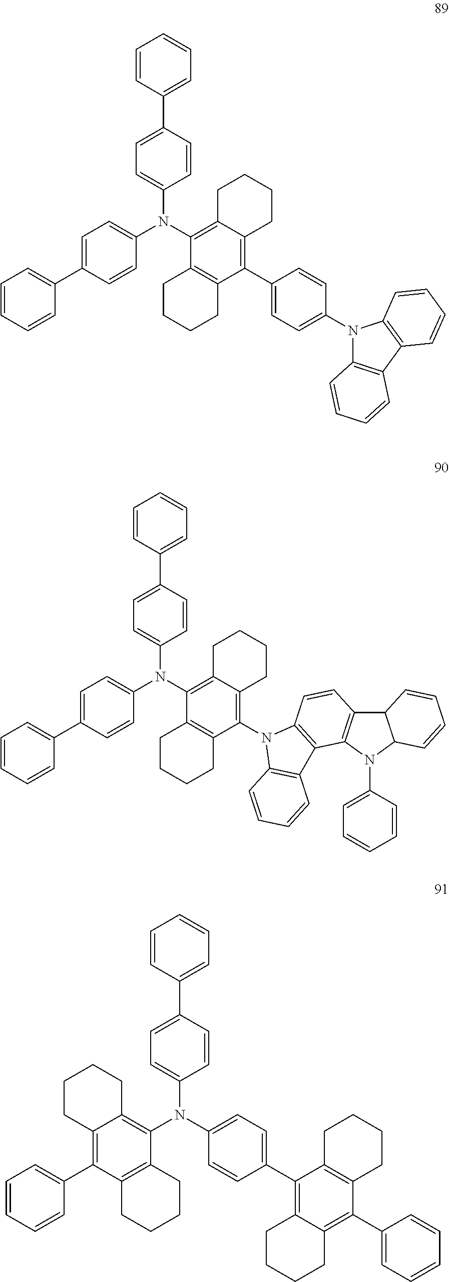 [SMILES: c1ccc(-c2ccc(N(c3ccc(-c4ccccc4)cc3)c3c4c(c(-c5ccc(-n6c7ccccc7c7ccccc76)cc5)c5c3CCCC5)CCCC4)cc2)cc1 ; C1=CC2c3ccc4c(c3N(c3ccccc3)C2C=C1)c1ccccc1n4-c1c2c(c(N(c3ccc(-c4ccccc4)cc3)c3ccc(-c4ccccc4)cc3)c3c1CCCC3)CCCC2 ; c1ccc(-c2ccc(N(c3ccc(-c4c5c(c(-c6ccccc6)c6c4CCCC6)CCCC5)cc3)c3c4c(c(-c5ccccc5)c5c3CCCC5)CCCC4)cc2)cc1]

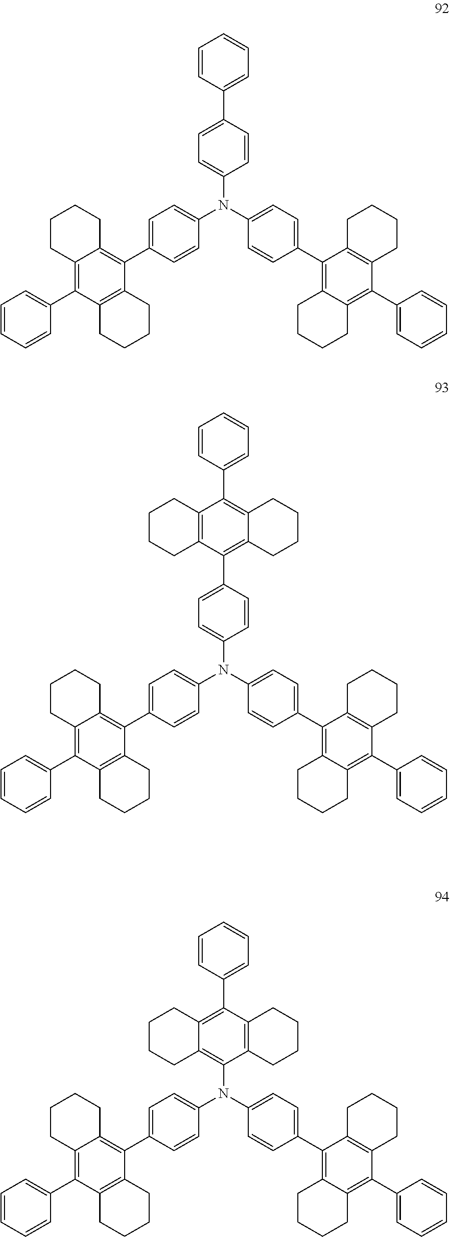 [SMILES: c1ccc(-c2ccc(N(c3ccc(-c4c5c(c(-c6ccccc6)c6c4CCCC6)CCCC5)cc3)c3ccc(-c4c5c(c(-c6ccccc6)c6c4CCCC6)CCCC5)cc3)cc2)cc1 ; c1ccc(-c2c3c(c(-c4ccc(N(c5ccc(-c6c7c(c(-c8ccccc8)c8c6CCCC8)CCCC7)cc5)c5ccc(-c6c7c(c(-c8ccccc8)c8c6CCCC8)CCCC7)cc5)cc4)c4c2CCCC4)CCCC3)cc1 ; c1ccc(-c2c3c(c(-c4ccc(N(c5ccc(-c6c7c(c(-c8ccccc8)c8c6CCCC8)CCCC7)cc5)c5c6c(c(-c7ccccc7)c7c5CCCC7)CCCC6)cc4)c4c2CCCC4)CCCC3)cc1]

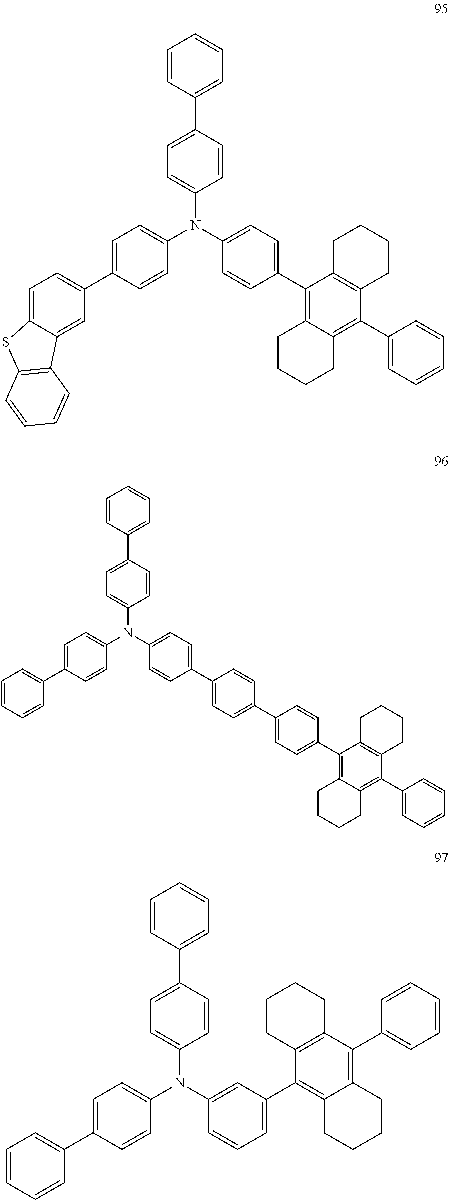 [SMILES: c1ccc(-c2ccc(N(c3ccc(-c4ccc5sc6ccccc6c5c4)cc3)c3ccc(-c4c5c(c(-c6ccccc6)c6c4CCCC6)CCCC5)cc3)cc2)cc1 ; c1ccc(-c2ccc(N(c3ccc(-c4ccccc4)cc3)c3ccc(-c4ccc(-c5ccc(-c6c7c(c(-c8ccccc8)c8c6CCCC8)CCCC7)cc5)cc4)cc3)cc2)cc1 ; c1ccc(-c2ccc(N(c3ccc(-c4ccccc4)cc3)c3cccc(-c4c5c(c(-c6ccccc6)c6c4CCCC6)CCCC5)c3)cc2)cc1]

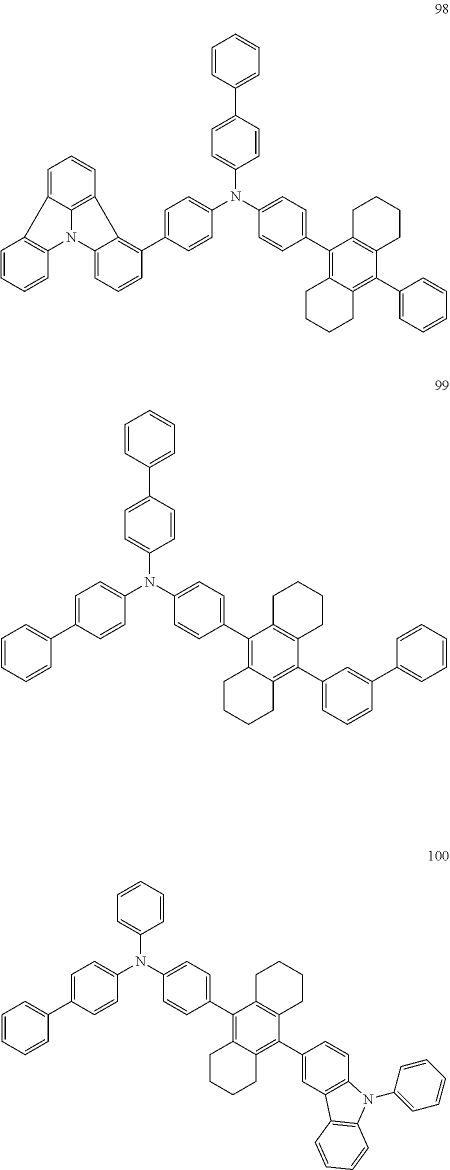 [SMILES: c1ccc(-c2ccc(N(c3ccc(-c4c5c(c(-c6ccccc6)c6c4CCCC6)CCCC5)cc3)c3ccc(-c4cccc5c4c4cccc6c7ccccc7n5c64)cc3)cc2)cc1 ; c1ccc(-c2ccc(N(c3ccc(-c4ccccc4)cc3)c3ccc(-c4c5c(c(-c6cccc(-c7ccccc7)c6)c6c4CCCC6)CCCC5)cc3)cc2)cc1 ; c1ccc(-c2ccc(N(c3ccccc3)c3ccc(-c4c5c(c(-c6ccc7c(c6)c6ccccc6n7-c6ccccc6)c6c4CCCC6)CCCC5)cc3)cc2)cc1]

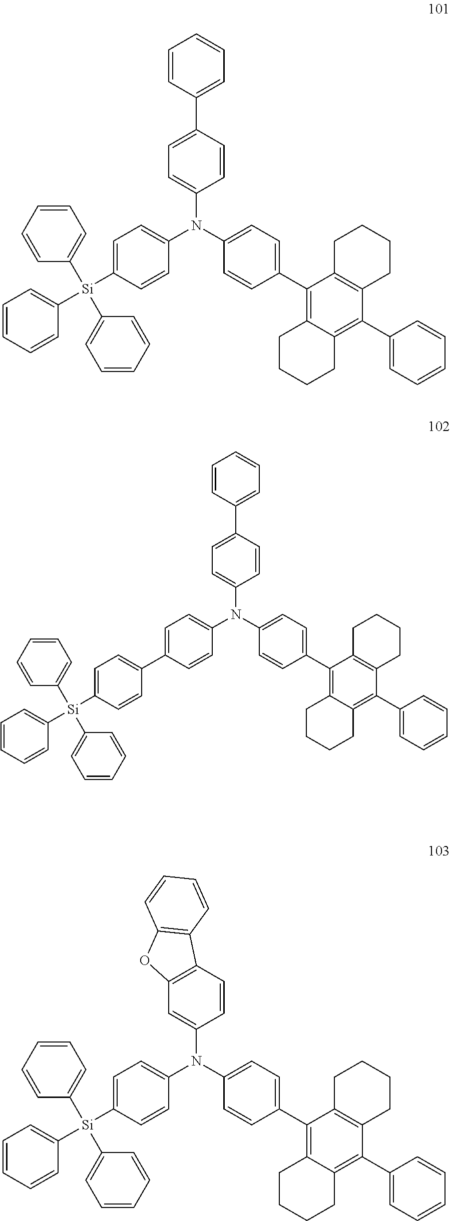 [SMILES: c1ccc(-c2ccc(N(c3ccc(-c4c5c(c(-c6ccccc6)c6c4CCCC6)CCCC5)cc3)c3ccc([Si](c4ccccc4)(c4ccccc4)c4ccccc4)cc3)cc2)cc1 ; c1ccc(-c2ccc(N(c3ccc(-c4ccc([Si](c5ccccc5)(c5ccccc5)c5ccccc5)cc4)cc3)c3ccc(-c4c5c(c(-c6ccccc6)c6c4CCCC6)CCCC5)cc3)cc2)cc1 ; c1ccc(-c2c3c(c(-c4ccc(N(c5ccc([Si](c6ccccc6)(c6ccccc6)c6ccccc6)cc5)c5ccc6c(c5)oc5ccccc56)cc4)c4c2CCCC4)CCCC3)cc1]

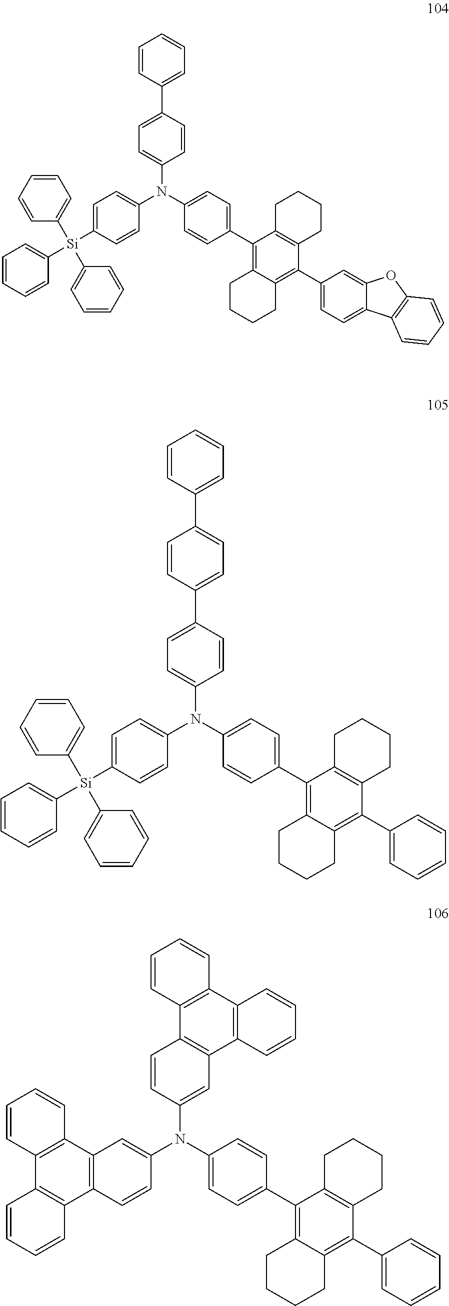 [SMILES: c1ccc(-c2ccc(N(c3ccc(-c4c5c(c(-c6ccc7c(c6)oc6ccccc67)c6c4CCCC6)CCCC5)cc3)c3ccc([Si](c4ccccc4)(c4ccccc4)c4ccccc4)cc3)cc2)cc1 ; c1ccc(-c2ccc(-c3ccc(N(c4ccc(-c5c6c(c(-c7ccccc7)c7c5CCCC7)CCCC6)cc4)c4ccc([Si](c5ccccc5)(c5ccccc5)c5ccccc5)cc4)cc3)cc2)cc1 ; c1ccc(-c2c3c(c(-c4ccc(N(c5ccc6c7ccccc7c7ccccc7c6c5)c5ccc6c7ccccc7c7ccccc7c6c5)cc4)c4c2CCCC4)CCCC3)cc1]

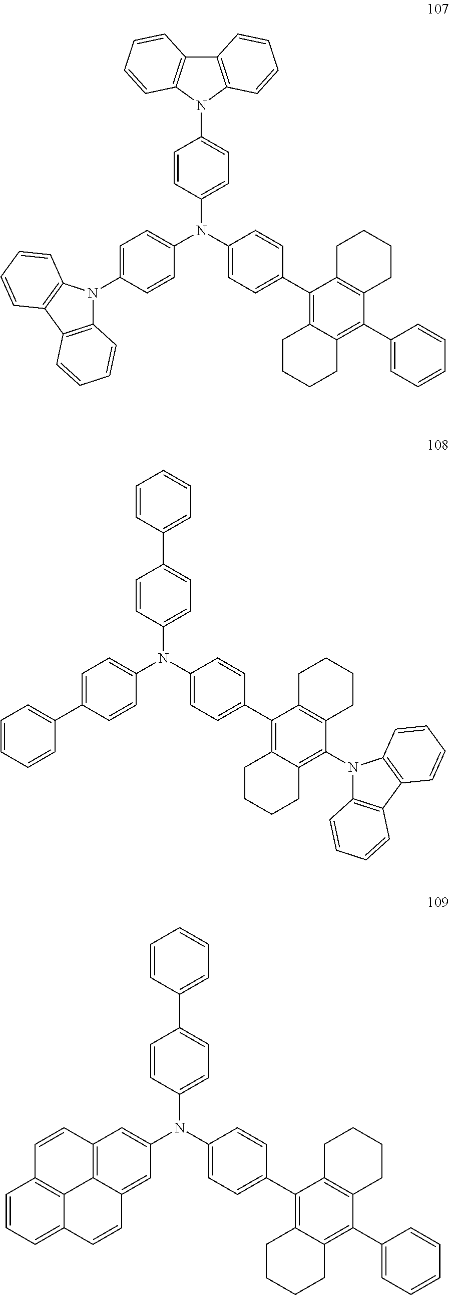 [SMILES: c1ccc(-c2c3c(c(-c4ccc(N(c5ccc(-n6c7ccccc7c7ccccc76)cc5)c5ccc(-n6c7ccccc7c7ccccc76)cc5)cc4)c4c2CCCC4)CCCC3)cc1 ; c1ccc(-c2ccc(N(c3ccc(-c4ccccc4)cc3)c3ccc(-c4c5c(c(-n6c7ccccc7c7ccccc76)c6c4CCCC6)CCCC5)cc3)cc2)cc1 ; c1ccc(-c2ccc(N(c3ccc(-c4c5c(c(-c6ccccc6)c6c4CCCC6)CCCC5)cc3)c3cc4ccc5cccc6ccc(c3)c4c56)cc2)cc1]

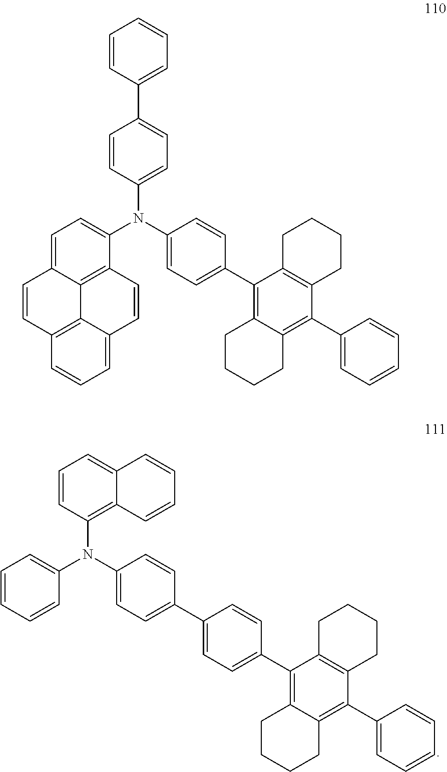 [SMILES: c1ccc(-c2ccc(N(c3ccc(-c4c5c(c(-c6ccccc6)c6c4CCCC6)CCCC5)cc3)c3ccc4ccc5cccc6ccc3c4c56)cc2)cc1 ; c1ccc(-c2c3c(c(-c4ccc(-c5ccc(N(c6ccccc6)c6cccc7ccccc67)cc5)cc4)c4c2CCCC4)CCCC3)cc1]

7. An organic electroluminescence (EL) device comprising the material for an organic EL device as claimed in claim 1.

8. An organic electroluminescence (EL) device comprising the material for an organic EL device as claimed in claim 1 in a layer of stacked layers between an emission layer and an anode.
